(19) United States
(12) Patent Application Publication
(10) Pub. No.: US 2024/0175025 A1
(43) Pub. Date: May 30, 2024

(54) ANTISENSE OLIGONUCLEOTIDES THAT BIND TO EXON 51 OF HUMAN DYSTROPHIN PRE-MRNA

(71) Applicant: The Governors of the University of Alberta, Edmonton (CA)

(72) Inventors: Toshifumi YOKOTA, Edmonton (CA); Yusuke ECHIGOYA, Kanagawa (JP)

(21) Appl. No.: 18/391,433

(22) Filed: Dec. 20, 2023

Related U.S. Application Data
(63) Continuation of application No. 17/466,833, filed on Sep. 3, 2021, now Pat. No. 11,891,603.
Application No. 17/466,833 is a continuation of application No. 16/632,827, filed on Jan. 21, 2020, now Pat. No. 11,142,767.

(30) Foreign Application Priority Data
Jul. 21, 2017 (GB) 1711809.2

(51) Int. Cl. C12N 15/113; A61P 21/00

(52) U.S. Cl. CPC C12N 15/113; A61P 21/00; C12N 2310/11; C12N 2310/3513; C12N 2320/33

(57) ABSTRACT

The present invention relates to a therapeutic antisense oligonucleotide which binds to exon 51 of the human dystrophin pre-mRNA to induce exon skipping, and conjugates and compositions thereof for the treatment of DMD.

### CROSS-REFERENCE TO RELATED APPLICATIONS

This application is continuation of U.S. patent application Ser. No. 17/466.833, filed Sep. 3, 2021, which is a continuation of U.S. patent application Ser. No. 16/632,827, filed on Jan. 21, 2020, now issued as U.S. Pat. No. 11,142,767, issued on Oct. 12, 2021, which is a U.S. National Phase Entry of International Application No. PCT/CA2018/050881. filed on Jul. 20, 2018, which claims the benefit of foreign priority to Application No. GB 1711809.2, filed Jul. 21, 2017, each of which is incorporated herein by reference in its entirety.

### SEQUENCE LISTING

The instant application contains a Sequence Listing which has been filed electronically in XML format and is hereby incorporated by reference in its entirety. Said XML copy, created on Dec. 15, 2023, is named 45532-746_302_SL.xml and is 568,263 bytes in size.

### BACKGROUND OF THE INVENTION

Disruption of alternative splicing underlies many diseases, and modulation of splicing using antisense oligonucleotides can have therapeutic implications. Splice-switching antisense oligonucleotides (SSOs) are emerging treatments for neuromuscular diseases, with several SSOs currently undergoing clinical trials for conditions such as spinal muscular atrophy (SMA) and Duchenne muscular dystrophy (DMD), where antisense-mediated exon skipping can restore the open reading frame and allow the synthesis of partly or wholly functional proteins instead of non-functional ones.

Duchenne muscular dystrophy (DMD) is one of the most prevalent lethal genetic disorders in boys worldwide, with an incidence of approx. 1 in 3,600-9,337 live male births. DMD is caused by the absence of dystrophin protein due to mutations in the dystrophin (DMD) gene. The gene encoding the protein contains 79 exons spread out over more than 2 million nucleotides of DNA. Any exonic mutation that changes the reading frame of the exon, or introduces a stop codon, or is characterized by removal of an entire out of frame exon or exons or duplications of one or more exons has the potential to disrupt production of functional dystrophin, resulting in DMD. A less severe form of muscular dystrophy, Becker muscular dystrophy (BMD) has been found to arise where a mutation, typically a deletion of one or more exons, results in a correct reading frame along the entire dystrophin transcript, such that translation of mRNA into protein is not prematurely terminated. If the joining of the upstream and downstream exons in the processing of a mutated dystrophin pre-mRNA maintains the correct reading frame of the gene, the result is an mRNA coding for a protein with a short internal deletion that retains some activity resulting in a Becker Page I Header phenotype. Deletions of an exon or exons which do not alter the reading frame of a dystrophin protein give rise to a BMD phenotype, whereas an exon deletion that causes a frame-shift will give rise to DMD (Monaco, Bertelson et al. 1988). In general, dystrophin mutations including point mutations and exon deletions that change the reading frame and thus interrupt proper protein translation result in DMD.

Currently one of the most promising therapeutic avenues is exon skipping using antisense oligonucleotides (AOs). Exon skipping can restore the reading frame by removing the mutant exon and/or its flanking exon(s) from the DMD pre-mRNA, enabling the production of truncated but partly-functional dystrophin protein. A majority of DMD patients harbour deletion mutations and 20% of these are amenable to exon 51 skipping.

In September 2016, the US Food and Drug Administration (FDA) conditionally approved the first DMD antisense drug, eteplirsen (Exondys 51), which was developed to exclude exon 51 from mutant DMD. Eteplirsen is an AO modified with a phosphorodiamidate morpholino oligomer (morpholino or PMO), an antisense chemistry that has been well-established in terms of its safety and effectiveness. However, eteplirsen remains controversial as there is only weak evidence supporting the effectiveness of the drug. both in terms of restoring dystrophin protein to therapeutically beneficial levels, and improving clinical outcomes. The FDA has previously rejected another drug candidate for DMD exon 51 skipping: the 2′-O-methyl-phosphorothioate-based AO ‘drisapersen’. Although therapeutics must ensure the highest possible benefit for the lowest amount of risk. no significant improvements in muscle function were demonstrated upon treatment with drisapersen, and its use led to concerns over safety.

Therefore, exon skipping therapies currently face a major challenge in that their observed efficacy in patients has been very low despite the fact that significant therapeutic effects have been demonstrated in many animal studies.

Exon skipping efficiency is largely dependent on the AO target sequence: however. there has been little debate or discussion that the sequences targeted by eteplirsen and drisapersen might not be the optimal choices for exon skipping therapy. Several groups have undertaken large-scale AO screening efforts to determine effective AO sequences computationally and empirically. However, the exon skipping effectiveness of designed AOs has not been evaluated both quantitatively and statistically. Although restoring dystrophin protein expression is necessary to improve dystrophic muscle function, the ability of AOs to rescue dystrophin protein expression has not been reported with sufficient Page 2 Header methods of quantification in previous AO screening studies. Other studies have highly relied on RT-PCR from primary DMD muscle cells. It is remarkable that the AO sequences of eteplirsen and drisapersen were determined only within this context.

Thus, the effectiveness of exon 51 skipping therapy could be improved by selecting more optimal AO sequences, and by performing more rigorous AO screening using a more reliable and direct biological measure—such as rescued dystrophin protein in DMD—for validating the best antisense oligonucleotides to be taken forward in a clinical trial.

It is an aim of one or more aspects of the present invention to address one or more such problems in the art.

### SUMMARY OF THE INVENTION

According to a first aspect of the present invention, there is provided an antisense oligonucleotide capable of binding to exon 51 of human dystrophin pre-mRNA, wherein binding of the antisense oligonucleotide takes place entirely within the region between 0) and +89 of the pre-mRNA sequence, and wherein the antisense oligonucleotide comprises at least 27 bases.

According to a second aspect of the present invention, there is provided a conjugate comprising an antisense oligonucleotide according to the first aspect and a carrier, wherein the carrier is conjugated to the antisense oligonucleotide.

According to a third aspect of the present invention, there is provided a cell loaded with a conjugate of the second aspect.

According to a fourth aspect of the present invention, there is provided a pharmaceutical composition comprising an antisense oligonucleotide according to the first aspect, and/or a conjugate according to the second aspect, and a pharmaceutically acceptable excipient. According to a fifth aspect of the present invention, there is provided a method of treating a muscular disorder in a subject, comprising administering an effective amount of an antisense oligonucleotide capable of binding to exon 51 of human dystrophin pre-mRNA to a subject, wherein binding of the antisense oligonucleotide takes place entirely within the region between 0 and +89 of the pre-mRNA sequence, and wherein the antisense oligonucleotide comprises at least 27 bases.

According to a sixth aspect of the present invention, there is provided an antisense oligonucleotide capable of binding to exon 51 of human dystrophin pre-mRNA for use in antisense oligonucleotide takes place entirely within the region between 0 and +89 of the pre-mRNA sequence, and wherein the antisense oligonucleotide comprises at least 27 bases.

According to a seventh aspect of the present invention, there is provided a method of increasing human dystrophin protein expression in a cell comprising contacting the cell with an effective amount of an antisense oligonucleotide capable of binding to exon 51 of human dystrophin pre-mRNA, wherein binding of the antisense oligonucleotide takes place entirely within the region between 0 and +89 of the pre-mRNA sequence, and wherein the antisense oligonucleotide comprises at least 27 bases.

### DETAILED DESCRIPTION OF THE INVENTION

The inventors have developed a series of antisense oligonucleotides binding within the early region at 0 to +89 of exon 51 of the dystrophin pre-mRNA sequence and which have a longer than usual length of at least 27 bases, each having remarkable efficiency and effectiveness.

In order to produce the antisense oligonucleotides, the inventors performed a study which quantitatively evaluated the effectiveness of morpholino-based antisense oligonucleotides for exon 51 skipping using a systematic screening method involving in silico, in vitro, and in vivo tests.

The inventors carried out a combination screening using a computational analysis to predict exon skipping efficiency of designed antisense oligonucleotide sequences followed by in vitro tests of morpholino antisense oligonucleotides in immortalized DMD patient-derived muscle cell lines. This research revealed that the beginning of the human dystrophin exon 51 sequence is a very promising target region for inducing exon skipping, specifically the region of 0 to +89 of the sequence. This is notably different from the internal region targeted by the known eteplirsen and drisapersen antisense therapies.

The antisense oligonucleotides identified from this region were then optimised for the most effective restoration of dystrophin production in muscle cells. Various factors were investigated, including the length of the antisense oligonucleotides. Surprisingly, the inventors found that antisense oligonucleotides binding in this early region are more effective when they are longer than many of the known antisense oligonucleotide sequences against exon 51. Specifically, the inventors identified an upward trend correlating effectiveness with the length of the antisense oligonucleotide from 27 bases and Page 4 Header longer. The inventors have shown that just a few bases difference means the antisense oligonucleotide has a significantly different efficiency. As demonstrated herein, 30-mer antisense oligonucleotides work up to 1.5-fold better than a 25-mer of the same sequence (42% vs.65% skipping efficiency). Without wishing to be bound by theory, this may be because longer sequences can be more specific to the target sequence and less likely to cause off-target effects.

It is demonstrated herein that the inventors' optimisation of these identified antisense oligonucleotide sequences has enabled efficiency in exon 51 skipping and in rescuing dystrophin protein to increase by up to more than 12-fold and 7-fold respectively compared to the industry standard ‘eteplirsen’ sequence. Furthermore, statistically significant in vivo exon 51 skipping by the most effective antisense oligonucleotide identified through these in vitro screenings was confirmed using transgenic mice harbouring the human DMD gene, which has never been shown for the eteplirsen or drisapersen antisense oligonucleotides. Accordingly, the antisense oligonucleotides described herein are shown to provide an effective therapy and treatment for muscular disorders, especially for the treatment of DMD. These antisense oligonucleotides are not only providing an alternative therapy into a field of medicine in which only one such drug has been approved for market. They also provide an improved option for treatment which is several times more effective at increasing dystrophin protein expression. This is expected to provide a viable option for treatment for those suffering from DMD and other muscular disorders with strong evidence to support the effectiveness of the therapy.

For the avoidance of doubt, and in order to clarify the way in which the present disclosure is to be interpreted, certain terms used in accordance with the present invention will now be defined further.

The invention includes any combination of the aspects and features described except where such a combination is clearly impermissible or expressly avoided.

It is noted that where aspects of the invention may refer methods or uses including an antisense oligonucleotide, this may also include a conjugate or pharmaceutical composition comprising an antisense oligonucleotide as defined herein.

The section headings used herein are for organisational purposes only and are not to be construed as limiting the subject matter described.

### Antisense Oligonucleotide

The present invention relates to antisense oligonucleotides having a length of at least 27 bases that bind to exon 51 of human dystrophin pre-mRNA within the region of 0 to +89 which can be used to treat muscular disorders.

Suitably, ‘antisense oligonucleotides’ may be referred to herein as ‘AOs’ or ‘oligos’ or ‘oligomers’.

Suitably the antisense oligonucleotide induces skipping of exon 51 of the human dystrophin gene.

Suitably the antisense oligonucleotide increases skipping of exon 51 of the human dystrophin gene.

Suitably the antisense oligonucleotide allows expression of functional human dystrophin protein.

Suitably the antisense oligonucleotide increases expression of functional human dystrophin protein.

Suitably, the antisense oligonucleotide comprises at least 28 bases, suitably at least 29 bases, suitably at least 30 bases.

Suitably, the antisense oligonucleotide comprises between 27 and 30 bases. In one embodiment, the antisense oligonucleotide comprises 30 bases.

In one embodiment, the antisense oligonucleotide consists of 30 bases.

Suitably, the binding of the antisense oligonucleotide takes place entirely within the region between 0 and +88, 0 and +87, 0 and +86, 0 and +85, 0 and +84, 0 and +83, 0 and +82, 0 and +81, 0 and +80, 0 and +79, 0 and +78 of the pre-mRNA sequence.

In one embodiment, the binding of the antisense oligonucleotide takes place entirely within the region between 0 and +78 of the pre-mRNA sequence.

Suitably, the antisense oligonucleotide comprises at least 27 bases of one of the following sequences: SEQ ID NO.1 (Ac0), SEQ ID NO.2 (Ac5), SEQ ID NO.3 (Ac26), SEQ ID NO.4 (Ac30), or SEQ ID NO.5 (Ac48).

Suitably, the antisense oligonucleotide comprises at least 28 bases of one of the following sequences: SEQ ID NO.1 (Ac0), SEQ ID NO.2 (Ac5), SEQ ID NO.3 (Ac26), SEQ ID NO.4 (Ac30), or SEQ ID NO.5 (Ac48).

Suitably, the antisense oligonucleotide comprises at least 29 bases of one of the following sequences: SEQ ID NO.1 (Ac0), SEQ ID NO.2 (Ac5), SEQ ID NO.3 (Ac26), SEQ ID NO.4 (Ac30), or SEQ ID NO.5 (Ac48).

Suitably, the antisense oligonucleotide comprises at least 27 contiguous bases of one of the following sequences: SEQ ID NO.1 (Ac0), SEQ ID NO.2 (Ac5), SEQ ID NO.3 (Ac26), SEQ ID NO.4 (Ac30), or SEQ ID NO.5 (Ac48).

Suitably, the antisense oligonucleotide comprises at least 28 contiguous bases of one of the following sequences: SEQ ID NO.1 (Ac0), SEQ ID NO.2 (Ac5), SEQ ID NO.3 (Ac26), SEQ ID NO.4 (Ac30), or SEQ ID NO.5 (Ac48).

Suitably, the antisense oligonucleotide comprises at least 29 contiguous bases of one of the following sequences: SEQ ID NO.1 (Ac0), SEQ ID NO.2 (Ac5), SEQ ID NO.3 (Ac26), SEQ ID NO.4 (Ac30), or SEQ ID NO.5 (Ac48).

Suitably, the antisense oligonucleotide shares at least 70%, at least 80%, at least 90%, at least 95%, at least 96%, at least 97%, at least 98%, or at least 99% identity with one of the following sequences: SEQ ID NO.1 (Ac0), SEQ ID NO.2 (Ac5), SEQ ID NO.3 (Ac26), SEQ ID NO.4 (Ac30), or SEQ ID NO.5 (Ac48).

Suitably, the antisense oligonucleotide shares between 90% and 100% identity with one of the following sequences: SEQ ID NO.1 (Ac0), SEQ ID NO.2 (Ac5), SEQ ID NO.3 (Ac26), SEQ ID NO.4 (Ac30), or SEQ ID NO.5 (Ac48).

Suitably, the antisense oligonucleotide shares at least 70%, at least 80%, at least 90%, at least 95%, at least 96%, at least 97%, at least 98%, or at least 99% homology with one of the following sequences: SEQ ID NO.1 (Ac0), SEQ ID NO.2 (Ac5), SEQ ID NO.3 (Ac26), SEQ ID NO.4 (Ac30), or SEQ ID NO.5 (Ac48).

Suitably, the antisense oligonucleotide shares between 90% and 100% homology with one of the following sequences: SEQ ID NO.1 (Ac0), SEQ ID NO.2 (Ac5), SEQ ID NO.3 (Ac26), SEQ ID NO.4 (Ac30), or SEQ ID NO.5 (Ac48).

Suitably, the antisense oligonucleotide may comprise a variant antisense oligonucleotide which differs from one of the following sequences: SEQ ID NO. 1 (Ac0). SEQ ID NO.2 (Ac5), SEQ ID NO.3 (Ac26), SEQ ID NO.4 (Ac30), or SEQ ID NO.5 (Ac48) by: 1, 2, 3, 4, 5, 6, 7, 8, 9, or 10 bases.

Suitably, the antisense oligonucleotide may comprise a variant antisense oligonucleotide which differs from one of the following sequences: SEQ ID NO.1 (Ac0), SEQ ID NO.2 (Ac5), SEQ ID NO.3 (Ac26), SEQ ID NO.4 (Ac30), or SEQ ID NO.5 (Ac48) by up to 3 bases. Suitably, the antisense oligonucleotide may comprise a variant antisense oligonucleotide which differs from one of the following sequences: SEQ ID NO.1 (Ac0), SEQ ID NO.2 (Ac5), SEQ ID NO.3 (Ac26), SEQ ID NO.4 (Ac30), or SEQ ID NO.5 (Ac48) by up to 2 bases. Suitably, the antisense oligonucleotide may comprise a variant antisense oligonucleotide which differs from one of the following sequences: SEQ ID NO.1 (Ac0). SEQ ID NO.2 (Ac5), SEQ ID NO.3 (Ac26), SEQ ID NO.4 (Ac30), or SEQ ID NO.5 (Ac48) by a single base. Suitably, the antisense oligonucleotide comprises one of the following sequences: SEQ ID NO.1 (Ac0), SEQ ID NO.2 (Ac5), SEQ ID NO.3 (Ac26), SEQ ID NO.4 (Ac30), or SEQ ID NO.5 (Ac48).

Suitably, the antisense oligonucleotide consists of one of the following sequences: SEQ ID NO.1 (Ac0), SEQ ID NO.2 (Ac5), SEQ ID NO.3 (Ac26), SEQ ID NO.4 (Ac30), or SEQ ID NO.5 (Ac48).

Suitably, the antisense oligonucleotide comprises SEQ ID NO.1 (Ac0) or SEQ ID NO.5 (Ac48).

In one embodiment, the antisense oligonucleotide comprises SEQ ID NO.1 (Ac0).

Suitably, the antisense oligonucleotide consists of SEQ ID NO.1 (Ac0) or SEQ ID NO.5 (Ac48).

In one embodiment, the antisense oligonucleotide consists of SEQ ID NO.1 (Ac0).

It will be appreciated that the invention may further include aspects directed towards each of the individual antisense oligonucleotide sequences listed in Table 3 i.e. an antisense oligonucleotide comprising or consisting of any of the sequences listed in Table 3.

Furthermore, in accordance with the second aspect of the invention, a conjugate comprising an antisense oligonucleotide as listed in Table 3 is envisaged. Furthermore a pharmaceutical composition in accordance with the fourth aspect of the invention,

comprising an antisense oligonucleotide as listed in Table 3 or a conjugate thereof is envisaged. Furthermore a medical use in accordance with the fifth aspect of the invention, comprising an antisense oligonucleotide as listed in Table 3 for the treatment of a muscular disorder is envisaged. Furthermore a method of treatment in accordance with the sixth aspect comprising an antisense oligonucleotide as listed in Table 3 is envisaged. Furthermore a method of increasing human dystrophin protein expression in a cell in accordance with the seventh aspect comprising an antisense oligonucleotide as listed in Table 3 is envisaged.

Suitably, the antisense oligonucleotide is synthetic, and non-natural.

Suitably, the antisense oligonucleotide may be routinely made through the well-known technique of solid phase synthesis. Equipment for such synthesis is sold by several manufacturers including, for example, Applied Biosystems (Foster City, Calif.). One method for synthesising oligonucleotides on a modified solid support is described in U.S. Pat. No. 4,458,066. Any other means for such synthesis known in the art may additionally or alternatively be employed.

Suitably, the antisense oligonucleotide is an antisense oligonucleotide analogue.

Suitably, the term ‘oligonucleotide analogue’ and ‘nucleotide analogue’ refer to any modified synthetic analogues of oligonucleotides or nucleotides respectively that are known in the art.

Suitable examples of oligonucleotide analogues include peptide nucleic acids (PNAs), morpholino oligonucleotides, phosphorothioate oligonucleotides, phosphorodithioate oligonucleotides, alkylphosphonate oligonucleotides, acylphosphonate oligonucleotides and phosphoramidite oligonucleotides.

Suitably, the antisense oligonucleotide comprises morpholino subunits. Suitably therefore, the antisense oligonucleotide is a morpholino antisense oligonucleotide.

Suitably, the antisense oligonucleotide comprises morpholino subunits linked together by phosphorus-containing linkages. Suitably therefore, the antisense oligonucleotide is a phosphoramidate or phosphorodiamidate morpholino antisense oligonucleotide.

The terms ‘morpholino antisense oligonucleotide’ or ?MO' (phosphoramidate or phosphorodiamidate morpholino oligonucleotide) refer to an antisense oligonucleotide Page 9 Header analog composed of morpholino subunit structures, where (i) the structures are linked together by phosphorus-containing linkages, suitably one to three atoms long, suitably two atoms long, and suitably uncharged or cationic, joining the morpholino nitrogen of one subunit to a 5′ exocyclic carbon of an adjacent subunit, and (ii) each morpholino ring bears a purine or pyrimidine base-pairing moiety effective to bind, by base specific hydrogen bonding, to a base in a polynucleotide.

Suitably, the antisense oligonucleotide comprises phosphorus-containing intersubunit linkages joining a morpholino nitrogen of one subunit to a 5′ exocyclic carbon of an adjacent subunit.

Suitably, the antisense oligonucleotide comprises phosphorus-containing intersubunit linkages in accordance with the following structure (I):

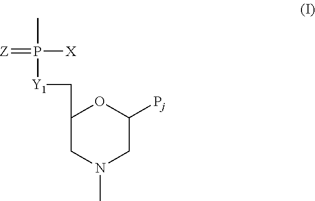

wherein:

Y1 is 0, S, NH—, or —CH2—;

Z is 0 or S;

Pj is a purine or pyrimidine base-pairing moiety effective to bind, by base-specific hydrogen bonding, to a base in a polynucleotide; and X is fluoro, optionally substituted alkyl, optionally substituted alkoxy, optionally substituted thioalkoxy, amino, optionally substituted alkylamino, or optionally substituted heterocyclyl.

Optionally, variations can be made to the intersubunit linkage as long as the variations do not interfere with binding or activity. For example, the oxygen attached to phosphorus may be substituted with sulfur (thiophosphorodiamidate). The 5′ oxygen may be substituted with amino or lower alkyl substituted amino. The pendant nitrogen attached to the phosphorus may be unsubstituted, monosubstituted, or disubstituted with (optionally substituted) lower alkyl.

Suitably, the synthesis, structures, and binding characteristics of morpholino oligonucleotides are detailed in U.S. Pat. Nos. 5,698,685, 5,217,866, 5,142,047, 5,034,506, 5,166,315, 5,521,063, and 5,506,337, and PCT Appn. No. PCT/US07/11435.

### Binding of the Antisense Oligonucleotide

The present invention relates to an antisense oligonucleotide capable of binding within the region 0 to +89 of exon 51 of human dystrophin pre-mRNA.

By ‘capable of binding’ it is meant that the antisense oligonucleotide comprises a sequence with is able to bind to human dystrophin pre-mRNA in the region stated.

Suitably, the antisense oligonucleotide is complementary to a sequence of human dystrophin pre-mRNA in the region stated.

Suitably, the antisense oligonucleotide comprises a sequence which is complementary to a sequence of human dystrophin pre-mRNA in the region stated.

The antisense oligonucleotide and a sequence within the region 0 to +89 of exon 51 of human dystrophin pre-mRNA are complementary to each other when a sufficient number of corresponding positions in each molecule are occupied by nucleotides which can hydrogen bond with each other and thereby cause exon skipping, suitably exon skipping of exon 51. Thus, ‘hybridisable’ and ‘complementary’ are terms which are used to indicate a sufficient degree of complementarity or pairing such that stable and specific binding occurs between the antisense oligonucleotide and a sequence within region 0 to +89 of exon 51 of human dystrophin pre-mRNA. Suitably, the antisense oligonucleotide is sufficiently hybridisable and/or complementary to a sequence within region 0 to +89 of exon 51 of human dystrophin pre-mRNA to induce exon skipping, suitably exon skipping of exon 51. Suitably, the antisense oligonucleotide may not be 100% complementary to a sequence within region of 0 to +89 of exon 51 of human dystrophin pre-mRNA. However, suitably the antisense oligonucleotide is sufficiently complementary to avoid non-specific binding.

Suitably, the antisense oligonucleotide is at least 70%, at least 80%, at least 90%, at least 95%, at least 96%, at least 97%, at least 98% at least 99% complementary to a sequence within the region 0 to +89 of exon 51 of human dystrophin pre-mRNA.

It is understood that in order for the antisense oligonucleotide to be capable of binding, it does not require that the entire length of the antisense oligonucleotide binds to the human dystrophin pre-mRNA. It will be appreciated that a portion of the antisense oligonucleotide Page 11 Header may not bind to the human dystrophin pre-mRNA, for example the 5′ or the 3′ ends of the antisense oligonucleotide. However, in accordance with the first aspect, the parts of the antisense oligonucleotide which are bound to the human dystrophin pre-mRNA must fall within the region of 0 to +89 of exon 51.

Suitably, therefore, the antisense oligonucleotide is hybridisable to a sequence within the region of 0 to +89 of exon 51 of human dystrophin pre-mRNA. Suitably, the antisense oligonucleotide is sufficiently hybridisable to a sequence within the region of 0 to +89 of exon 51 of human dystrophin pre-mRNA to cause exon skipping of exon 51.

### Human Dystrophin

The present invention relates to a therapeutic antisense oligonucleotide for use in the treatment of muscular disorders, particularly dystrophin disorders such as DMD.

Dystrophin is a rod-shaped cytoplasmic protein, and a vital part of the protein complex that connects the cytoskeleton of a muscle fibre to the surrounding extracellular matrix through the cell membrane.

Dystrophin protein contains multiple functional domains. The DMD gene, encoding the dystrophin protein, is one of the longest known human genes covering 2.3 megabases (0.08% of the human genome) at locus Xp21. The primary transcript in muscle measures about 2.100 kilobases and takes 16 hours to transcribe: the mature mRNA measures 14.0 kilobases. The 79-exon muscle transcript codes for a protein of 3685 amino acid residues. Dystrophin protein contains an actin binding domain and a central rod domain. This large central domain is formed by 24 spectrin-like triple-helical elements of about 109 amino acids, which have homology to alpha-actinin and spectrin. The repeats are typically interrupted by four proline-rich non-repeat segments, also referred to as hinge regions. Each repeat is encoded by two exons, typically interrupted by an intron between amino acids 47 and 48 in the first part of alpha-helix 2. The other intron is found at different positions in the repeat, usually scattered over helix 3. Dystrophin also contains a cysteine-rich domain including a cysteine-rich segment (i.e., 15 Cysteines in 280 amino acids).

In normal cases, the amino-terminus of dystrophin binds to F-actin and the carboxy-terminus binds to the dystrophin-associated protein complex (DAPC) at the sarcolemma. The DAPC includes the dystroglycans, sarcoglycans, integrins and caveolin, and mutations in any of these components cause autosomally inherited muscular dystrophies. Normal skeletal muscle tissue contains only small amounts of dystrophin (about 0.002% of total muscle protein), but its absence (or abnormal expression) leads to the development of a Page 12 Header severe and currently incurable symptoms most readily characterized by several aberrant intracellular signaling pathways that ultimately yield pronounced myofiber necrosis as well as progressive muscle weakness and fatigability. The DAPC is destabilized when dystrophin is absent, which results in diminished levels of the member proteins, and in turn leads to progressive fibre damage and membrane leakage. In various forms of muscular dystrophy, such as Duchenne's muscular dystrophy (DMD) and Becker's muscular dystrophy (BMD), muscle cells produce an altered and functionally defective form of dystrophin, or no dystrophin at all, mainly due to mutations in the gene sequence that lead to incorrect splicing. The predominant expression of the defective dystrophin protein, or the complete lack of dystrophin or a dystrophin-like protein, leads to rapid progression of muscle degeneration.

The mRNA encoding dystrophin in muscular dystrophy patients typically contains out-of-frame mutations (e.g. deletions, insertions or splice site mutations), resulting in frameshift or early termination of the translation process, so that in most muscle fibres no functional dystrophin is produced.

Suitably, the antisense oligonucleotide triggers exon skipping to restore the reading frame of the dystrophin mRNA. Suitably, the antisense oligonucleotide triggers exon skipping of exon 51 to restore the reading frame of the dystrophin mRNA. Suitably, restoration of the reading frame restores production of a partially functional dystrophin protein.

Suitably, the partially functional dystrophin is a truncated dystrophin protein.

Suitably, the truncated dystrophin protein is the same dystrophin protein produced in patients suffering from the less severe muscular disorder; BMD.

### Muscular Disorder

The present invention relates to the use of therapeutic antisense oligonucleotides in the treatment of muscular disorders.

Suitably the muscular disorder is selected from any muscular disorder resulting from a genetic mutation.

Suitably the muscular disorder is selected from any muscular disorder resulting from a genetic mutation in a gene associated with muscle function.

Suitably the muscular disorder is selected from any muscular disorder resulting from a genetic mutation in the human dystrophin gene.

Suitably, the muscular disorder is selected from any muscular dystrophy disorder.

Suitably, the muscular disorder is selected from Duchenne muscular dystrophy, Becker muscular dystrophy, congenital muscular dystrophy, Distal muscular dystrophy, Emery-Dreifuss muscular dystrophy, Facioscapulohumeral muscular dystrophy, Limb-girdle muscular dystrophy, Myotonic muscular dystrophy, Oculopharyngeal Muscular dystrophy. Suitably, the muscular disorder is Duchenne Muscular Dystrophy (DMD) or Becker Muscular Dystrophy (BMD).

In one embodiment, the muscular disorder is DMD.

### Carrier and Conjugate

The present invention also relates to a conjugate of the antisense oligonucleotide with a carrier.

Suitably, the carrier may comprise any molecule operable to transport the antisense oligonucleotide into a target cell, suitably into a muscle cell.

Suitable carriers may include: peptides, small molecule chemicals, polymers, nanoparticles, lipids, liposomes, exosomes or the like.

Suitably, the carrier is a peptide. The peptide may be selected from viral proteins such as VP22 (derived from herpes virus tegument protein), snake venom protein such as CyLOP-1 (derived from crotamin), cell adhesion glycoproteins such as pVEC (derived from murine vascular endothelial-cadherin protein), Penetratin (Antennapedia homeodomain), Tat (human immunodeficiency virus transactivating regulatory protein) or reverse Tat, for example. Suitably, the peptide is a cell penetrating peptide.

Suitably, the peptide is an arginine-rich cell penetrating peptide.

The use of arginine-rich peptide carriers is particularly useful. Certain arginine based peptide carriers have been shown to be highly effective at delivery of antisense compounds into primary cells including muscle cells (Marshall, Oda et al. 2007; Jearawiriyapaisarn, Moulton et al. 2008: Wu, Moulton et al. 2008). Furthermore, compared to other peptides,

the arginine peptide carriers when conjugated to an antisense oligonucleotide, demonstrate an enhanced ability to alter splicing of several gene transcripts (Marshall, Oda et al. 2007). Suitably, the arginine-rich cell penetrating peptide may be selected from those carrier peptides described in WO2015075747, WO2013030569, WO2009147368, US20120289457, or US20160237426, for example.

In one embodiment, the arginine rich cell penetrating peptide is selected from those described in WO2013030569 or WO2009147368.

Suitably, the carrier has the capability of inducing cell penetration of the antisense oligonucleotide within at least 30%, 40%, 50%, 60%, 70%, 80%, 90%, or 100% of cells of a given cell culture population. Suitably, the carrier has the capability of inducing cell penetration of the antisense oligonucleotide within at least 30%, 40%, 50%, 60%, 70%, 80%, 90%, or 100% of muscle cells in a muscle cell culture.

Suitably, conjugation of the carrier to the antisense oligonucleotide may be at any position suitable for forming a covalent bond between the carrier and the antisense oligonucleotide or between the linker moiety and the antisense oligonucleotide. For example, conjugation of a carrier may be at the 3′ end of the antisense oligonucleotide. Alternatively, conjugation of a carrier to the antisense oligonucleotide may be at the 5′ end of the oligonucleotide. Alternatively, a carrier may be conjugated to the antisense oligonucleotide through any of the intersubunit linkages.

Suitably, the carrier is covalently coupled at its N-terminal or C-terminal residue to the 3′ or 5′ end of the antisense oligonucleotide.

Suitably, the carrier is coupled at its C-terminal residue to the 5′ end of the antisense oligonucleotide.

Optionally, where the antisense oligonucleotide comprises phosphorus-containing intersubunit linkages, and the carrier is a peptide, the peptide may be conjugated to the antisense oligonucleotide via a covalent bond to the phosphorous of the terminal linkage group.

Alternatively, when the carrier is a peptide, and the antisense oligonucleotide is a morpholino, the peptide may be conjugated to the nitrogen atom of the 3′ terminal morpholino group of the oligomer.

Optionally, the carrier may be conjugated to the antisense oligonucleotide via a linker moiety. Optionally, the linker moiety may comprise one or more of: an optionally substituted piperazinyl moiety, a beta alanine, glycine, proline, and/or a 6-aminohexanoic acid residue in any combination.

Alternatively, the carrier may be conjugated directly to the antisense oligonucleotide without a linker moiety.

Suitably the conjugate may further comprise a homing moiety.

Suitably, the homing moiety is selective for a selected mammalian tissue, i.e., the same tissue being targeted by the antisense oligonucleotide. Suitably, the homing moiety is selective for muscle tissue.

Suitably, the homing moiety is a homing peptide.

Suitable homing peptides are disclosed in ‘Effective Dystrophin Restoration by a Novel Muscle-Homing Peptide-Morpholino Conjugate in Dystrophin-Deficient mdx Mice’ Gao et Mol Ther. 2014 July; 22(7): 1333-1341, for example.

Suitably, the carrier peptide and the homing peptide may be formed as a chimeric fusion protein.

Suitably, the conjugate may comprise a chimeric peptide formed from a cell penetrating peptide and a muscle-specific homing peptide.

Optionally, the conjugate may be of the form: carrier peptide-homing peptide-antisense oligonucleotide or of the form: homing peptide-carrier peptide-antisense oligonucleotide. Suitably, the antisense oligonucleotide may be conjugated to a carrier that enhances the solubility of the antisense oligonucleotide. Suitably the solubility in an aqueous medium. Suitably, a carrier that enhances solubility may be conjugated to the antisense oligonucleotide in addition to a carrier operable to transport the antisense oligonucleotide. Suitably, the carrier that enhances solubility and the carrier that transports the antisense oligonucleotide may be formed as a chimeric fusion protein.

Suitable carriers that enhance the solubility of an antisense oligonucleotide are polymers, such as polyethylene glycol, or triethylene glycol.

### Pharmaceutically Acceptable Excipient

The present invention further relates to a pharmaceutical composition comprising the antisense oligonucleotide of the invention or a conjugate thereof, further comprising one or more pharmaceutically acceptable excipients.

Suitably, the pharmaceutical composition is prepared in a manner known in the art (as described in Remingtons Pharmaceutical Sciences, Mack Publ. Co., Easton, PA (1985)), with pharmaceutically inert inorganic and/or organic excipients being used. The term “pharmaceutically acceptable” refers to molecules and compositions that are physiologically tolerable and do not typically produce an allergic or similarly untoward reaction when administered to a patient.

Suitably, the pharmaceutical composition may be formulated as a pill, tablet, coated tablet, hard gelatin capsule, soft gelatin capsule and/or suppository, solution and/or syrup, injection solution, microcapsule, implant and/or rod, and the like.

In one embodiment, the pharmaceutical composition may be formulated as an injection solution.

Suitably, pharmaceutically acceptable excipients for preparing pills, tablets, coated tablets and hard gelatin capsules may be selected from any of: Lactose, corn starch and/or derivatives thereof, talc, stearic acid and/or its salts, etc.

Suitably, pharmaceutically acceptable excipients for preparing soft gelatin capsules and/or suppositories may be selected from fats, waxes, semisolid and liquid polyols, natural and/or hardened oils, etc.

Suitably, pharmaceutically acceptable excipients for preparing solutions and/or syrups may be selected from water, sucrose, invert sugar, glucose, polyols, etc.

Suitably, pharmaceutically acceptable excipients for preparing injection solutions may be selected from water, saline, alcohols, glycerol, polyols, vegetable oils, etc.

Suitably, pharmaceutically acceptable excipients for preparing microcapsules, implants and/or rods may be selected from mixed polymers such as glycolic acid and lactic acid or the like.

In addition, the pharmaceutical composition may comprise a liposome formulation which are described in N. Weiner, (Drug Develop Ind Pharm 15 (1989) 1523), “Liposome Dermatics” (Springer Verlag 1992) and Hayashi (Gene Therapy 3 (1996) 878).

Optionally, the pharmaceutical composition may comprise two or more different antisense oligonucleotides or conjugates thereof. Optionally, the pharmaceutical composition may further comprise one or more antisense oligonucleotides or conjugates thereof targeting different exons, suitably different exons of the human dystrophin pre-mRNA. Optionally, the one or more further antisense oligonucleotides or conjugates thereof may target exons adjacent to exon 51, for example, exon 50 or exon 52 of the human dystrophin pre-mRNA. Suitably, the one or more antisense oligonucleotides or conjugates thereof targeting different exons of the human dystrophin pre-mRNA are operable, together with the antisense oligonucleotide of the invention, to restore the reading frame of dystrophin mRNA.

Optionally, the pharmaceutical composition may further comprise one or more antisense oligonucleotides or conjugates thereof targeting different genes. For example, the one or more further antisense oligonucleotides or conjugates thereof may target myostatin. Such dual targeting is described in Dual exon skipping in myostatin and dystrophin for Duchenne muscular dystrophy' Kemaladewi et al. BMC Med Genomics. 2011 Apr 20:4:36.

Optionally, the one or more further antisense oligonucleotides may be joined together and/or joined to the antisense oligonucleotide of the first aspect.

Optionally, the antisense oligonucleotide and/or conjugate may be present in the pharmaceutical composition as a physiologically tolerated salt. Suitably, physiologically tolerated salts retain the desired biological activity of the antisense oligonucleotide and/or conjugate thereof and do not impart undesired toxicological effects. For antisense oligonucleotides, suitable examples of pharmaceutically acceptable salts include (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like: (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like: and (d) salts formed from elemental anions such as chlorine, bromine, and iodine.

Optionally, the pharmaceutical composition may comprise, in addition to at least one antisense oligonucleotide and/or conjugate, one or more different therapeutically active ingredients. The one or more therapeutically active ingredients may be selected from, for example: corticosteroids, utrophin-upregulators, TGF-beta inhibitors, and myostatin inhibitors.

Suitably, in addition to the active ingredients and excipients, a pharmaceutical composition may also comprise additives, such as fillers, extenders, disintegrants, binders, lubricants, wetting agents, stabilizing agents, emulsifiers, preservatives, sweeteners, dyes, flavorings or aromatizing agents, thickeners, diluents or buffering substances, and, in addition, solvents and/or solubilizing agents and/or agents for achieving a slow release effect, and also salts for altering the osmotic pressure, coating agents and/or antioxidants. Suitable additives may include Tris-HCl, acetate, phosphate, Tween 80, Polysorbate 80, ascorbic acid, sodium metabisulfite, Thimersol, benzyl alcohol, lactose, mannitol, or the like.

### Administration

The present invention relates to a therapeutic antisense oligonucleotide and to a pharmaceutical composition comprising the therapeutic antisense oligonucleotide which are for administration to a subject.

Suitably, the antisense oligonucleotide and/or pharmaceutical composition may be for topical, enteral or parenteral administration.

Suitably, the antisense oligonucleotide and/or pharmaceutical composition may be for administration orally, transdermally, intravenously, intrathecally, intramuscularly, subcutaneously, nasally, transmucosally or the like.

In one embodiment, the antisense oligonucleotide and/or pharmaceutical composition is for intramuscular administration.

In one embodiment, the antisense oligonucleotide and/or pharmaceutical composition is for intramuscular administration by injection.

An ‘effective amount’ or ‘therapeutically effective amount’ refers to an amount of the antisense oligonucleotide, administered to a subject, either as a single dose or as part of a Page 19 Header series of doses, which is effective to produce a desired physiological response or therapeutic effect in the subject.

Suitably, the desired physiological response includes increased expression of a relatively functional or biologically active form of the dystrophin protein, suitably in muscle tissues or cells that contain a defective dystrophin protein or no dystrophin.

Suitably, the desired therapeutic effects include improvements in the symptoms or pathology of a muscular disorder, reducing the progression of symptoms or pathology of a muscular disorder, and slowing the onset of symptoms or pathology of a muscular disorder. Examples of such symptoms include fatigue, mental retardation, muscle weakness, difficulty with motor skills (e.g., running, hopping, jumping), frequent falls, and difficulty walking.

Suitably, the antisense oligonucleotide or conjugate thereof are administered at a dose in the range from about 0.0001 to about 100 mg per kilogram of body weight per day.

Suitably, the antisense oligonucleotide or conjugate thereof are administered daily, once every 2, 3, 4, 5, 6, 7, 8, 9, 10, 11, 12, 13, 14 days, once every 1, 2, 3, 4, 5, 6, 7, 8, 9, 10, 11, 12 weeks, or once every 1, 2, 3, 4, 5, 6, 7, 8, 9, 10, 11, 12 months.

Suitably, the dose and frequency of administration may be decided by a physician, as needed, to maintain the desired expression of a functional dystrophin protein.

Suitably, the antisense oligonucleotide or conjugate thereof may be administered as two, three, four, five, six or more sub-doses separately at appropriate intervals throughout the day, optionally, in unit dosage forms.

### Subject

The present invention also relates to treatment of a muscular disorder by administering a therapeutically effective amount of the antisense oligonucleotide or conjugate thereof to a subject in need thereof.

Suitably the subject has a muscular disorder, as defined above. Suitably, the subject is mammalian. Suitably the subject is human.

Suitably the subject may be male or female. However, suitably the subject is male.

Suitably, the subject is any age. However, suitably the subject is between the ages of 1 month old to 50 years old, suitably between the ages of 1 years old and 30 years old, suitably between the ages of 2 years old to 27 years old, suitably between the ages of 4 years old to 25 years old

### Increased Exon Skipping and Dystrophin Expression

The present invention relates to a therapeutic antisense oligonucleotide for use in the treatment of muscular disorder by inducing exon skipping in the human dystrophin pre-mRNA to restore functional dystrophin protein expression.

Suitably, a ‘functional’ dystrophin protein refers to a dystrophin protein having sufficient biological activity to reduce the progressive degradation of muscle tissue that is otherwise characteristic of muscular dystrophy when compared to the defective form of dystrophin protein that is present in subjects with a muscular disorder such as DMD.

Suitably, a functional dystrophin protein may have about 10%, 20%, 30%, 40%, 50%, 60%, 70%, 80%, 90%, or 100% of the in vitro or in vivo biological activity of wild-type dystrophin. Suitably, a functional dystrophin protein has at least 10% to 20% of the in vitro or in vivo biological activity of wild-type dystrophin.

Suitably, the activity of dystrophin in muscle cultures in vitro can be measured according to myotube size, myofibril organization, contractile activity, and spontaneous clustering of acetylcholine receptors (see, e.g., Brown et al., Journal of Cell Science. 112:209-216, 1999).

Animal models are also valuable resources for studying the pathogenesis of disease, and provide a means to test dystrophin-related activity. Two of the most widely used animal models for DMD research are the mdx mouse and the golden retriever muscular dystrophy (GRMD) dog, both of which are dystrophin negative (see, e.g., Collins & Morgan, Int J Exp Pathol 84: 165-172, 2003). These and other animal models can be used to measure the functional activity of various dystrophin proteins.

Suitably, ‘exon skipping’ refers to the process by which an entire exon, or a portion thereof, is removed from a given pre-processed RNA (pre-mRNA), and is thereby excluded from being present in the mature RNA that is translated into a protein.

Suitably, the portion of the protein that is otherwise encoded by the skipped exon is not present in the expressed form of the protein.

Suitably therefore, exon skipping creates a truncated, though still functional, form of the protein as defined above.

Suitably, the exon being skipped is an exon from the human dystrophin gene, which may contain a mutation or other alteration in its sequence that otherwise causes aberrant splicing.

Suitably, the exon being skipped is exon 51 of the dystrophin gene.

Suitably, the antisense oligonucleotide is operable to induce exon skipping in dystrophin pre-mRNA.

Suitably, the antisense oligonucleotide is operable to induce exon skipping of exon 51 in dystrophin pre-mRNA.

Suitably, the antisense oligonucleotide is operable to increase expression of a functional form of a dystrophin protein in muscle tissue, and is operable to increase muscle function in muscle tissue.

Suitably, the antisense oligonucleotide is operable to increase muscle function by at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% compared to muscle function in subjects with a muscular disorder such as DMD that have not received the antisense oligonucleotide.

Suitably, the antisense oligonucleotide is operable to increase the percentage of muscle fibres that express a functional dystrophin protein in about at least 1%₅ 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% of muscle fibres compared to subjects with a muscular disorder such as DMD that have not received the antisense oligonucleotide.

Suitably, the antisense oligonucleotide is operable to induce expression of a functional form of a dystrophin protein to a level of at least 1, 2, 3, 4, 5, 6, 7, 8, 9, 10, 15, 20, 25, 30, 25, 40, 45, or 50% of the expression of dystrophin protein in wild type cells and/or subjects. Suitably, the antisense oligonucleotide is operable to induce expression of a functional form of a dystrophin protein to a level of at least 1, 2, 3, 4, 5, 6, 7, 8, 9, 10, 15, or 20% of the expression of dystrophin protein in wild type cells and/or subjects.

Suitably, the antisense oligonucleotide is operable to induce expression of a functional form of a dystrophin protein to a level of at least 10, 15, or 20% of the expression of dystrophin protein in wild type cells and/or subjects.

Suitably, the antisense oligonucleotide is operable to induce exon 51 skipping in the dystrophin pre-mRNA to a level of at least 10%, 20%, 30%, 40%, 50%, 60%, 70%, 80%, 90%, or 100%.

Suitably, the antisense oligonucleotide is operable to induce exon 51 skipping in the dystrophin pre-mRNA to a level of at least 60%, 70%, 80%, 90%, or 100%.

Suitably, the antisense oligonucleotide is operable to induce exon 51 skipping in the dystrophin pre-mRNA to a level of between 60% to 80%.

An ‘increased’ or ‘enhanced’ amount may include an increase that is 1.1, 1.2, 1.5, 2, 2.5, 3, 4, 5, 6, 7, 8, 9, 10, 15, 20, 30, 40, 50 or more times the amount produced when no antisense oligonucleotide compound (the absence of an agent) or a control compound is administered under the same circumstances.

Suitably, an ‘increased’ or ‘enhanced’ amount is a statistically significant amount.

### BRIEF DESCRIPTION OF THE DRAWINGS

Certain embodiments of the present invention will now be described with reference to the following figures and tables in which:

FIG. 1 Shows in vitro screening of antisense oligonucleotides (AOs) and analog AOs of eteplirsen (aEte) and drisapersen (aDri) at 10 μM, in immortalized clonal exon 52-deleted DMD skeletal muscle cells (KM571). Differentiated myotubes were harvested at day 5 following transfection (A) Efficiency of exon 51 skipping as measured by one-step RT-PCR. Representative images are shown. M, 100 bp marker: blank, no RNA template. (B) Efficiency in inducing truncated dystrophin protein as measured by quantitative Western blotting with the anti-dystrophin C-terminal antibody. Rescued dystrophin protein levels are calculated using calibration curves with healthy 8220 cells. Data represent mean +SD from 3-4 independent experiments. **p<0.01 vs aEte. t p<0.05 and tt p<0.01 vs aDri. §§ p<0.01 vs all of AOs in (A) and vs Ac0 in (B).

FIG. 2 Shows a time-course analysis of dystrophin exon 51 skipping and protein in an exon 52-deleted DMD-KM571 cell line transfected with AcO, Ac48, and analog AOs of Page 23 Header eteplirsen and drisapersen at 5 μM. Samples were collected at days 2 and 11 post-transfection (A) RT-PCR analysis of exon 51 skipping. M, 100 bp marker; R, replicate number: blank, no RNA templates. (B) Quantification of induced dystrophin protein by Western blotting with the anti-dystrophin C-terminal antibody. Representative replicates from 3 independent experiments are shown.

FIG. 3 Shows the dose-dependent effects of AcO, Ac48, and analogs AOs of eteplirsen and drisapersen in immortalized DMD skeletal muscle cells as measured by one-step RT-PCR and quantitative Western blotting. DMD skeletal muscle cells were transfected with AOs at 1, 3, and 10 μM and harvested at day 5 post-transfection. (A) and (B) show exon 51 skipping efficiency and expression levels of rescued dystrophin protein, respectively, in DMD muscle cells with exon 52 deletion mutation (ID KM 571). Efficacy of skipping exon 51 and rescuing dystrophin protein expression is shown in (C) and (D), respectively, in DMD muscle cells harboring exons 48-50) deletion mutation (ID 6594). Data represent mean ±SD from 3-7 independent experiments in the KM571 cell line and from 3-4 independent experiments in the 6594 cell line. *p<0.05. **p<0.01 vs aEte; t p<0.05 and tt p<0.01 vs Ac48: § p<0.05. §§ p<0.01 vs aDri in the same concentration, NS, no significance vs Ac0 at the next dose; ns, no significance vs Ac0 at 10 μM. (E) Dose-responsiveness to the AOs analysed by regression model. Statistical validity of regression equations in skipping and producing dystrophin protein was p<0.008 and p<0.014. respectively. Plots indicate values of exon skipping or dystrophin protein levels predicted in the regression analysis. The regression slope and 95% confidence interval (CI) are shown in individual AOs.

FIG. 4 Shows Immunocytochemistry in immortalized DMD patient-derived skeletal muscle cells with exon 52 (ID KM571) and exons 48-50 deletion mutations (ID 6594). Cells at day 5 post-transfection with 10 μM AcO, Ac48. and analog eteplirsen (aEte) were stained with anti-dystrophin C-terminal antibody. Grey lines indicate dystrophin-positive myotubes. White dots indicate nuclei counter-stained with DAPI. * indicates representative false-positive myotubes due to their contraction or detachment from the culture plate. Representative images are shown from 3 independent experiments. Scale bar: 100 μm.

FIG. 5 Shows Length optimization of the AcO morpholino antisense oligonucleotide. Immortalized DMD muscle cells were transfected with Ac0 morpholinos composed of 25-, 26-, 27-, 28-, 29-, and 30-mer lengths. A representative image and quantification of exon 51 skipping induced by Ac0 morpholinos at 1 μM (A and B) and 3 μM (C and D) in DMD

muscle cells with exon 52 deletion (KM571) are shown as represented by RT-PCR. (E-H) indicate the results in immortalized DMD cells with exons 48-50 deletion. The data are shown from 3 independent experiments.

FIG. 6 shows Exon 51 skipping efficiency induced by AcO, Ac48, analog AOs of eteplirsen (aEte) and drisapersen (aDri) in primary DMD and healthy skeletal muscle cells. Differentiated myotubes were transfected with AcO, Ac48, and analog eteplirsen and drisapersen at 10 μM, and then harvested 3 days later. Exon 51 skipping efficiency as represented by one-step RT-PCR was shown in primary DMD cells with the deletion mutation of exons 45-50 (ID 4546) (A and B) or exons 49-50 (ID 4555) (C and D), and primary healthy muscle cells (E and F). Data represent mean±SD from at least triplicate wells in each condition. M. 100 bp marker. *p<0.05 and **p<0.01 vs Ac48. tt p<0.01 vs aEte. §§<0.01 vs aDri.

FIG. 7 shows In vivo efficacy of 30-mer Aco antisense morpholino oligonucleotide in the hDMD/Dmd-null mouse model. Exon skipping efficacy was analysed by RT-PCR with tibialis anterior muscles 2 weeks after the intramuscular injection of Ac0 morpholino or analog eteplirsen, aEte (50 μg in 30 μL saline). (A) Densitometry analysis of exon 51 skipping as represented by a microchip-based capillary electrophoresis system. (B) Averaged percentage of exon 51 skipping efficiency (mean±SE). N=7 in each group. M, marker; NT, non-treated muscle, UM, upper marker dye; LM, lower marker dye.

FIG. 8 shows In vivo efficacy of 30-mer Ac48 antisense morpholino oligonucleotide in the hDMD/Dmd-null mouse model. Exon skipping efficacy was analysed by RT-PCR with tibialis anterior muscles 2 weeks after the intramuscular injection of Ac48 morpholino or analog drisapersen aDri (50 μg in 30 μL saline). (A) Densitometry analysis of exon 51 skipping as represented by a microchip-based capillary electrophoresis system. (B) Averaged percentage of exon 51 skipping efficiency (mean±SE). N=7 in each group. M, marker; NT, non-treated muscle, UM, upper marker dye; LM, lower marker dye.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or 27 process so disclosed, may be combined in any combination, except combinations where at least some of such features and/or steps are mutually exclusive.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed. The reader's attention is directed to all papers and documents which are filed concurrently with or previous to this specification in connection with this application and which are open to public inspection with this specification, and the contents of all such papers and documents are incorporated herein by reference.

### EXAMPLES

### Materials and methods

### 1.1 Design and In Silico Screening of AOs

413 30-mer and 25-mer AOs targeting exon 51 were designed and analysed using the AO predictive algorithm we recently developed (see Table 3). Table 3 shows in the columns from left to right: the exon number, the distance from acceptor splice site, the AO sequence (5′ to 3′), the predicted skipping %, and the ranking within the screen. The left hand AOs are 30mers and the right hand AOs are 25-mers. Based on predicted exon skipping efficiencies, 8 AOs spaced at least 4 bases apart were selected for in vitro screening (Table 2). Target sequence specificities of selected AOs, eteplirsen, and drisapersen were analysed using The University of California, Santa Cruz Genome Browser (http://genome.ucsc.edu/index.html), confirming that the AO sequences theoretically do not bind any non-target RNA sequences with 100% identity.

### 1.2 Antisense Morpholinos

All antisense sequences, including analog AOs of eteplirsen and drisapersen, were synthesized with the morpholino chemistry by Gene Tools (Philomath, OR).

### 1.3 Cells

Immortalized human skeletal muscle cells derived from three healthy subjects (IDs 8220, CHQ, and KM155) and two DMD patients harbouring deletion mutations of exon 52 (ID KM571) and exons 48-50 (ID 6594) in the DMD gene, respectively, were generated by transduction with human telomerase-expressing and cyclin-dependent kinase 4-expressing vectors in the Institute of Myology human cell immortalization platform, as previously described.33 The three immortalized healthy muscle cell lines were characterized and the clonal line 8220, which showed the highest dystrophin expression was selected as a positive control to prevent overestimation of rescued dystrophin expression in immortalized DMD cells. Primary skeletal muscle cells derived from DMD patients with deletion mutations of ex45-50 (ID 4546) and ex49-50 (ID 4555) and a healthy subject were prepared by the BioBank of Skeletal Muscle, Nerve Tissue, DNA and cell lines.

### 1.4 AO Transfection

To mimic as closely as possible the in vivo effects of AO-mediated exon skipping therapy, mature, differentiated myotubes expressing sufficient levels of DMD mRNA were used for in vitro screening. Cells were cultured in proliferation conditions with growth medium (GM): DMEM/F12 with skeletal muscle supplement mix (Promocell, Heidelberg, Germany), 20% fetal bovine serum (Life Technologies, Waltham, MA), and antibiotics (50 units penicillin and 50 μg/ml streptomycin, Life Technologies, Waltham, MA). Immortalized and primary DMD skeletal muscle cells were seeded at 1.7×104/cm2 and 2.2×104/cm2, respectively, in collagen type I-coated 12 or 24-well culture plates. Two days after seeding, at approximately 80-90% confluence, GM was replaced with differentiation medium (DM): DMEM/F12 with 2% horse serum (GE Healthcare, Chicago, IL), 1× ITS solution (Sigma, St. Louis, MO), and antibiotics. After three days in DM, cells were transfected with AO at 1, 3, 5 or 10 μM containing 6 μM of Endo-porter transfection reagent (Gene Tools, Philomath, OR) (concentrated AOs at 1 mM were incubated at 65° C. for 10 min just before diluting with DM). Two days following AO transfection, AO-containing DM was replaced with regular DM. Cells were harvested at day 2, 5, or 11 after AO transfection (day 5, 8 or 14 following differentiation).

### 1.5 Mice

Animal studies were approved by the Animal Care and Use Committee at the University of Alberta, Children's National Medical Center, and National Center of Neurology and Page 27 Header Psychiatry (NCNP). Male and female Dmd exon 52-deficient mdx5242 and wild-type mice (Jackson Laboratory, Bar Harbor, ME) with a C57BL/6J background were prepared at age 4-8 weeks. Dmd mutation in affected mice was confirmed by genotyping with PCR. A transgenic mouse model harboring the human DMD gene and lacking the mouse Dmd gene (hDMD/Dmd-null mouse) was generated by cross-breeding male hDMD mice (Jackson Laboratory, Bar Harbor, ME) with female Dmd-null mice.

### 1.6 Intramuscular Injection

Mouse version morpholinos of AcO, Ac48, eteplirsen or drisapersen at 5 or 20 μg in 40 μL saline were intramuscularly injected into tibialis anterior (TA) muscle under inhalation anesthesia with isoflurane as previously described.43 Fifty-μg of Ac0 morpholino and analog eteplirsen in 30 μL saline was injected into TA muscles of hDMD/Dmd-null mice. All muscle samples were harvested 2 weeks after intramuscular injection.

### 1.7 Exon Skipping Analysis by RT-PCR

Total RNA was extracted with Trizol (Invitrogen, Waltham, MA) as previously described. RT-PCR to detect dystrophin mRNA was performed with the SuperScript III One-Step RT-PCR System (Invitrogen, Waltham, MA) and 0.2 μM of forward and reverse primers (see Table 1) for 200 ng and 320 ng of total RNA in immortalized and primary skeletal muscle cells, respectively. Primers were designed using Primer3Plus software and their specificity was confirmed in healthy human skeletal muscle cells (line 8220). The RT-PCR conditions were as follows: 50° C. for 5 minutes: 94° C. for 2 minutes: 35 cycles at 94° C. for 15 seconds, 60° C. for 30 seconds, and 68° C. for 35 seconds: and 68° C. for 5 minutes. PCR products were separated on a 1.5% agarose gel and visualized by SYBR Safe DNA Gel Stain (Invitrogen, Waltham, MA). Using ImageJ software (NIH) or the MCE-202 MultiNA system (Shimadzu, Kyoto, Japan), the efficiency of exon 51 skipping was calculated using the following formula: exon 51-skipped transcript intensity/(native+intermediate+exon 51-skipped transcript intensities)×100 (%) Exsiskippedtranscript

Native+Exsiskippedtranscript^{xd(%)}. Unknown top bands above the native band, possibly coming from unexpected splicing events, were excluded from quantification of skipping efficiency. The sequences of the PCR products were confirmed with Big Dye Terminator v3.1 (Applied Biosystems, Waltham, MA). GAPDH or 18S ribosomal RNA was used as an internal control.

TABLE 1

| Name | Sequence (5′→3′) | Purpose |
|---|---|---|
| Ex49/50_94-10_hDMD_Fwd | CAGCCAGTGAAGAGGAAGTTAG | Immortal KM571 DMD cells with ex52 |
| SEQ ID NO. 16 | | del. |
| Ex53 80-99 hDMD_Rv | CCAGCCATTGTGTTGAATCC | Primary DMD and healthy cells |
| SEQ ID NO.-17 | | hDMDIDmd-null mice |
| Ex47 60-79 hDMD Fwd | AGGACCCGTGCTTGTAAGTG | Immortal 6594 DMD cells with ex48-50 |
| SEQ ID NO. 18 | | del. |
| Ex52_83-105_hDMD_Rv | GATTGTTCTAGCCTCTTGATTGC | Primary 4555 DMD cells with ex49-50 |
| SEQ ID NO. 19 | | del. |
| Ex43/44_167-12_hDMD_Fwd | GACAAGGGCGATTTGACAG | |
| SEQ ID NO. 20 | | |
| Ex52_83-105_hDMD_Rv | GATTGTTCTAGCCTCTTGATTGC | Primary 4546 DMD cells with ex45-50 |
| SEQ ID NO. 19 | | del. |
| Ex49/50 94-10 hDMD Fwd | CAGCCAGTGAAGAGGAAGTTAG | Primary healthy cells |
| SEQ ID NO. 16 | | |
| Ex52 83-105 hDMD Rv | GATTGTTCTAGCCTCTTGATTGC | hDMD/Dmd-null mice |
| SEQ ID NO. 19 | | |
| mDmd_ex49_83-102_Fwd | CAAGCACTCAGCCAGTGAAG | |
| SEQ ID NO. 21 | | |
| deletion mDmd_ex53_81-100_Rv | TCCAGCCATTGTGTTGAATC | mdx52 mice with ex52 |
| SEQ ID NO. 22 | | |
| hGAPDH_662-81_Fwd | TCCCTGAGCTGAACGGGAAG | |
| SEQ ID NO. 23 | | |
| control hGAPDH_860-79_Rv | GGAGGAGTGGGTGTCGCTGT | Internal |
| SEQ ID NO. 24 | | |
| h18S_760-82_Fwd | TCGATGCTCTTAGCTGAGTGTCC | |
| SEQ ID NO. 25 | | |
| control h18S_1039-58_Rv | TGATCGTCTTCGAACCTCCG | Internal |
| SEQ ID NO. 26 | | |

### 1.8 Western Blotting

Cells were harvested with RIPA buffer (Thermo Scientific, Waltham, MA) containing complete, Mini, EDTA-free protease inhibitor cocktail (Roche, Basel, Switzerland), and then homogenized by passing through a 21-gauge needle 10 times. The supernatants as loading samples were prepared by centrifugation at 14,000 g for 15 min at 4° C. Protein from muscle tissues were prepared as previously described. Protein concentrations were adjusted using the Bradford assay with supernatants diluted 100 times with distilled water. Proteins in a sample buffer containing 10% SDS, 70 mM Tris-HCl, pH 6.8, 5 mM EDTA, 20% glycerol, 0.004% bromophenol blue and 5% 2-mercaptoethanol were heated at 70° C. for 10 min. Western blotting was then done as previously described.32,43,44 Twelve-μg and thirty-μg from cells and tissues, respectively, were used for sodium dodecyl sulfate-polyacrylamide gel electrophoresis. Blots were incubated with a rabbit polyclonal antibody against dystrophin C-terminal (1:2500, ab15278, Abcam, Cambridge, United Kingdom) in the blocking solution or DYSI antibody against dystrophin rod domain (1:400, Leica Biosystems, Buffalo Grove, IL) for 1 hour at room temperature. The primary antibody was reacted with HRP-conjugated anti-rabbit or mouse IgG H+L antibody (1:10,000, Bio-Rad, Hercules, CA). Expression levels of dystrophin protein induced by AOs were quantified using calibration curves (R2=0.93−0.99) from dystrophin protein of healthy 8220 skeletal muscle cells diluted with protein from non-treated DMD cells, or wild-type mouse using ImageJ (NIH). Alpha-tubulin was detected on the same membrane as a loading control. Myosin heavy chain (MyHC) on post-transferred gels was stained with Coomassie Brilliant Blue (Bio-Rad, Hercules, CA) as a loading control/differentiation marker.

### 1.9 Immunocytochemistry

Cells were fixed with 4% paraformaldehyde for 5 min at room temperature. After washing with PBS containing 0.01% Triton X-100, cells were blocked with 10% goat serum (Life Technologies, Waltham, MA) in PBS with 0.05% Triton X-100 for 20 min and then incubated with anti-dystrophin C-terminal (ab15278) or rod-domain (DYS1) antibody at 1:50 dilution in blocking solution overnight at 4° C. Dystrophin signals were detected with Alexa 488- or 594-conjugated secondary antibody (1:500). Desmin (1:80, Abcam, Cambridge, United Kingdom) and MyHC-fast type (1:30, Leica Biosystems, Buffalo Grove, IL) were detected to confirm myogenic differentiation of cells. Cells were stored in Slow Fade Gold Antifade Mountant with DAPI (Invitrogen, Waltham, MA) at 4° C. until analysed.

### 1.10 Immunohistochemistry

Dystrophin-positive muscle fibers on cryosections from TA muscles of non-treated and treated mdx52 mice were detected with the ab15278 antibody as previously described. Signal intensity of dystrophin in the treated mice was compared with that in wild-type using neutral density filters (Eclipse TE 2000-U, Nikon, Tokyo, Japan).

### 1.11 Statistical Analysis

For determining the significance of efficiencies in exon skipping and dystrophin protein rescue, data sets were prepared from at least three independent experiments in immortalized cells, triplicate wells in primary cells, and 3-7 mice. The statistical analysis between AO-treated groups was performed by one-way ANOVA followed by a post hoc Tukey-Kramer multiple comparison test. Simple linear regression analysis was performed for dose-responsiveness to AOs. Statistical significance was set at p<0.05 for all analyses.

### 2. Results

### 2.1 In Silico Screening of AOs for Exon 51 Skipping

We designed total 413 AOs: 204 and 209 AOs with 30-mer and 25-mer lengths, respectively, which cover all possible target sites in DMD exon 51 (see Table 3). Our exon skipping efficiency algorithm (In Silico Screening Based on Predictive Algorithms as a Design Tool for Exon Skipping Oligonucleotides in Duchenne Muscular Dystrophy' Echigoya et al. PLOS ONE March 2015) predicted that the highest efficiency for exon 51 skipping was 80.5% for 30-mer AOs, and 41.2% for 25-mer AOs in the initial 5′ site of exon 51. In silico screening indicated a very low exon skipping efficiency for the 30-base region targeted by eteplirsen (23.7%), which was ranked 92nd in all 413 AO candidates tested. It is noted that the drisapersen target site is completely encompassed by that of the 30-mer eteplirsen.

### 2.2 Characterization of Immortalized Clonal Healthy and DMD Skeletal Muscle Cell Lines

Significant issues in preclinical testing with primary DMD muscle cells include low purity of muscle cells and insufficient amounts of mutant dystrophin mRNA, which present problems when trying to test AO efficacy. To overcome these hurdles, we generated immortalized clonal skeletal muscle cells from 3 healthy subjects and 2 DMD patients with exon 52 (ex52) and ex48-50 deletion (del.) mutations (IDs KM571 and 6594, respectively). All immortalized skeletal muscle cell lines tested expressed easily detectable dystrophin mRNA from day 3 Page 31 Header after induction of differentiation. To avoid overestimation of dystrophin protein levels induced by AOs in DMD cells, we selected a cell line (ID 8220) with the highest level of dystrophin protein among three immortalized healthy skeletal muscle cell lines as determined by Western blotting to serve as a positive control. Dystrophin protein expression in the 8220 cell line was also confirmed by immunocytochemistry.

### 2.3 In Vitro Screening of Exon 51 Skipping AOs

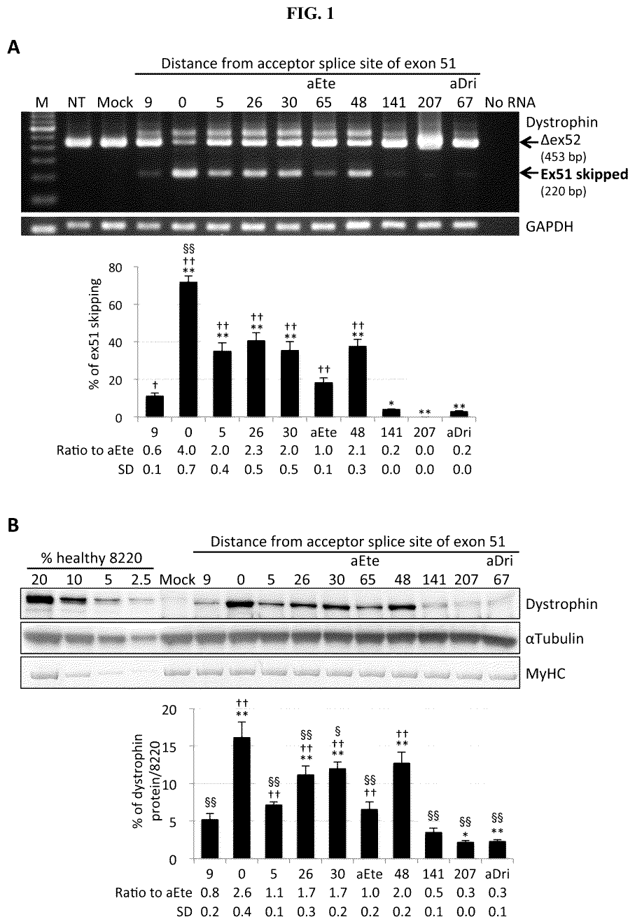

Based on the in silico screening results, we selected eight 30-mer AOs, including both high-ranking and low-ranking sequences, spaced at least 4 bases apart from each other for in vitro screening (Table 2). In the present study, all tested AOs, including eteplirsen and drisapersen sequences, were synthesized using the morpholino chemistry that has been demonstrated to be well-tolerated in patients enrolled in clinical trials. Here, we termed control morpholino oligonucleotides having the same sequences as eteplirsen and drisapersen (produced by Gene Tools) as “analog eteplirsen” and “analog drisapersen”. In RT-PCR, 5 of our morpholino AOs (AcO, Ac5, Ac26, Ac30 and Ac48) at 10 μM showed significantly higher skipping efficiency compared to analog eteplirsen and drisapersen in immortalized DMD skeletal muscle cells harboring ex52 del. (FIG. 1A). Of the tested AOs, Ac0 in particular had the highest skipping efficiency, reaching up to 72%, which was 4 and 25 times more efficient than analogs of eteplirsen and drisapersen, respectively. In Western blotting, Ac0 also induced the highest levels of dystrophin protein, reaching up to 16% of levels in the healthy control cell line, followed by Ac48 at 13% (FIG. 1B). Interestingly, the two AOs, Ac0 and Ac48, with the highest skipping efficiency when tested were not those predicted to be the best from the algorithm.

TABLE 2

| | | | Distance | Predicted | |
|---|---|---|---|---|---|
| Name | Oligo sequence (5′ to 3′) | Length (mer) | from Ac | Skip % | Ranking |
| hEx51_Ac9 | CCACAGGTTGTGTCACCAGAGTAACAGTCT | 30 | 9 | 80.5 | 1 |
| SEQ NO. 13 | | | | | |
| hEx51_Ac0 | GTGTCACCAGAGTAACAGTCTGAGTAGGAG | 30 | 0 | 80.1 | 2 |
| SEQ ID No. 1 | | | | | |
| hEx51_Ac5 | AGGTTGTGTCACCAGAGTAACAGTCTGAGT | 30 | 5 | 73.0 | 4 |
| SEQ ID NO. 2 | | | | | |
| hEx51_Ac26 | GGCAGTTTCCTTAGTAACCACAGGTTGTGT | 30 | 26 | 66.3 | 12 |
| SEQ ID NO. 3 | | | | | |
| hEx51_Ac30 | AGATGGCAGTTTCCTTAGTAACCACAGGTT | 30 | 30 | 55.5 | 25 |
| SEQ ID NO. 4 | | | | | |
| Eteplirsen | CTCCAACATCAAGGAAGATGGCATTTC TAG | 30 | 65 | 23.7 | 67 |
| SEQ ID NO. 6 | | | | | |
| hEx51_Ac48 | ATGGCATTTCTAGTTTGGAGATGGCAGTTT | 30 | 48 | 10.6 | 128 |
| SEQ ID NO. 5 | | | | | |
| hEx51_Ac141 | TTATAACTTGATCAAGCAGAGAAAGCCAGT | 30 | 141 | 1.8 | 142 |
| SEQ ID NO. 14 | | | | | |
| hEx51_Ac207 | atacCTTCTGCTTGATGATCATCTCGTTGA | 30 | 207 | NA | NA |
| SEQ ED NO. 15 | | | | | |
| Drisapersen | TCAAGGAAGATGGCATTTCT | 20 | 67 | NA | NA |
| SEQ ID NO. 7 | | | | | |
| hEx51_Ac0-29mer | TGTCACCAGAGTAACAGTCTGAGTAGGAG | 29 | 0 | NA | NA |
| SEQ ID NO. 8 | | | | | |
| hEx51_Ac0-28mer | GTCACCAGAGTAACAGTCTGAGTAGGAG | 28 | 0 | NA | NA |
| SEQ ID NO. 9 | | | | | |
| hEx51_Ac0-27mer | TCACCAGAGTAACAGTCTGAGTAGGAG | 27 | 0 | NA | NA |
| SEQ ID NO. 10 | | | | | |
| hEx51_Ac0-26mer | CACCAGAGTAACAGTCTGAGTAGGAG | 26 | 0 | NA | NA |
| SEQ ID NO. 11 | | | | | |
| hEx51_Ac0-25mer | ACCAGAGTAACAGTCTGAGTAGGAG | 25 | 0 | 33.3 | 10a |
| SEQ ID NO. 12 | | | | | |

Ac, acceptor splice site.
Uncapitalized nucleotides indicate intronic sequence.
a the ranking in 25-mer AOs.

### 2.4 Time-Course Analysis With AcO, Ac48, and Analog AOs of Eteplirsen and Drisapersen

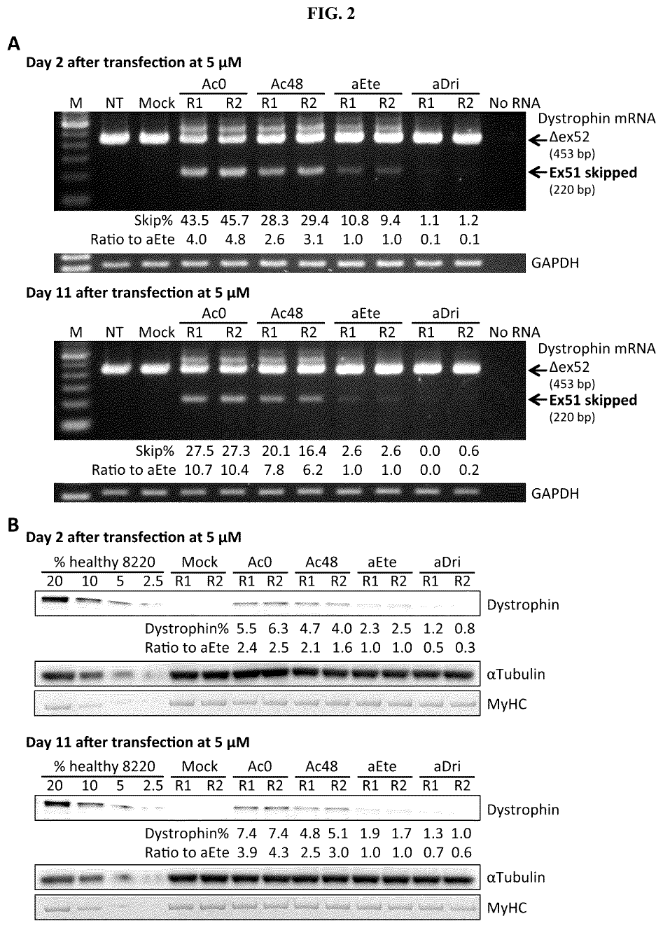

The persistent effects of AcO, Ac48, and analogs of eteplirsen and drisapersen at 5 μM were examined in ex52 del. KM571 cells. The superiority of the oligonucleotides Ac0 and Ac48 of the present invention, with respect to exon skipping efficiency and dystrophin protein rescue, was observed at days 2 and 11 post-transfection compared to analog AOs of eteplirsen and drisapersen (FIG. 2).

### 2.5 Dose-Dependent Effects of AcO, Ac48, and Analog Eteplirsen and Drisapersen

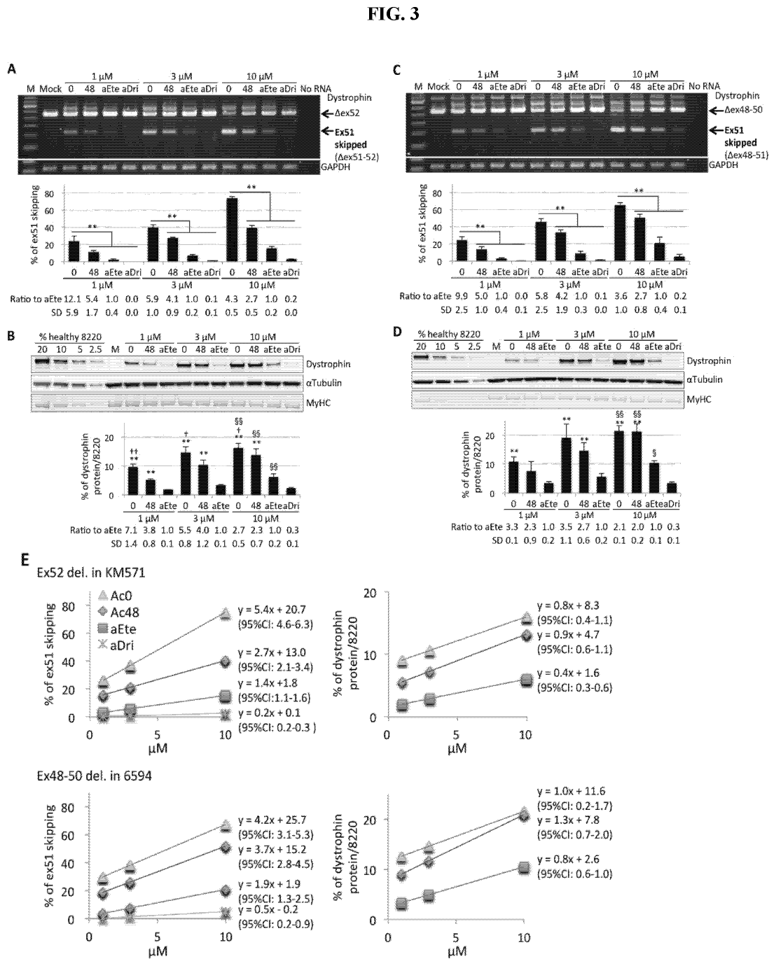

RT-PCR showed that Ac0 at the highest concentration of 10 μM induced up to 74% and 64% exon 51 skipping in DMD KM571 (ex52 del.) and 6594 cells (ex48-50 del.), respectively, which were significantly higher than analogs of eteplirsen and drisapersen (FIG. 3). At the lowest concentration (1 μM), Ac0 showed 12 and 10 times higher exon skipping efficiency compared to analog eteplirsen in KM571 and 6594 cells, respectively. Interestingly, even a concentration of 1 μM Ac0 induced higher levels of exon 51 skipping than 10 μM analog eteplirsen (24% efficiency vs 15% in KM571 and 24% efficiency vs 21% in 6594, respectively). Quantitative Western blotting revealed that 10 μM Ac0 rescued dystrophin protein expression in DMD cell lines up to 21% of healthy cell line levels (FIG. 3A to D). Even at 1 μM, the relative ratio of Ac0 to analog eteplirsen represented 7.1 and 3.3 times higher efficiency in producing dystrophin protein in KM571 and 6594 cell lines, respectively. Ac0 at 1 μM enabled the production of rescued dystrophin protein at higher or comparable levels than analog eteplirsen at 10 μM (10% vs 6% in KM571 and 11% vs 10% in 6594, respectively), confirming that Aco is more than 10-fold effective at producing Page 33 Header dystrophin protein compared to analog eteplirsen concentration-wise. Analog drisapersen did not work effectively for either of exon skipping or dystrophin production in the DMD muscle cell lines. The exon skipping response to Ac0 and Ac48 occurred in a dose-dependent manner that was greater than both analog eteplirsen, and analog drisapersen (FIG. 3A and C). The dose-responsiveness of AcO with respect to dystrophin protein production was also higher than the control analogs in both DMD cell lines (FIG. 3E).

### 2.6 Immunocytochemical Assessment of Dystrophin Protein Rescue

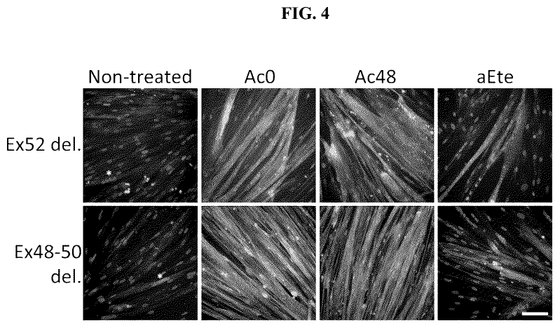

Immunocytochemistry revealed that Ac0 and Ac48 at 10 μM yielded more dystrophin-positive myotubes and displayed stronger signal intensity in DMD skeletal muscle cell lines harboring ex52 and ex48-50 del. mutations compared to analog eteplirsen (FIG. 4).

### 2.7 Length Optimization of Ac0 Morpholino

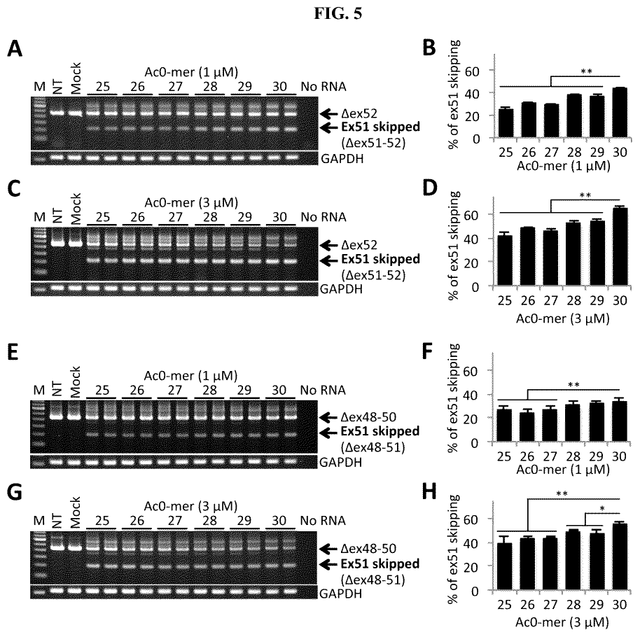

In silico and in vitro screening revealed that the initial 5′ region of exon 51 between 0 and +89 is an important region for influencing exon 51 skipping. To optimize the sequence length of Ac0 targeting this region, we compared the skipping efficiencies of Ac0 morpholinos of different lengths (25- to 30-mer), in which nucleotides at the 5′ site were systematically removed one at a time (see Table 2). In vitro testing in immortalized DMD muscle cells treated with 1 μM of these AOs showed that 25-30-mer Ac0 morpholinos produced efficient exon skipping (>20%) (FIG. 5), an effect that was not observed analog eteplirsen, and analog drisapersen at the same dose (FIG. 3). However, the efficiency of exon skipping increased as the length of the AO was increased. The statistically significant effectiveness of 30-mer Ac0 was confirmed at 1 and 3 μM doses compared to the shorter Aco morpholinos in both cell lines, even those AOs that are only 1 or 2 bases shorter.

### 2.8 Effect of AcO, Ac48, and Analog Eteplirsen and Drisapersen on Primary DMD Patient-Derived Skeletal Muscle Cells

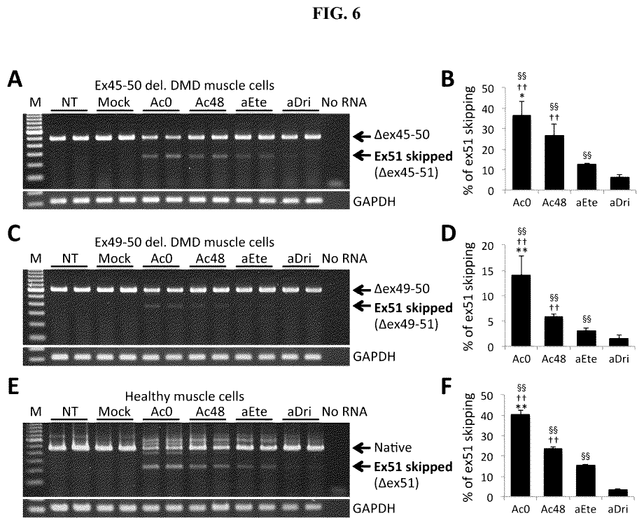

We also tested the AOs in primary DMD skeletal muscle cells with exons 45-50 (ID 4546) or exons 49-50 del. mutations (ID 4555) to validate if the superior efficacy of 30-mer Ac0 is consistent for other muscle cell types and deletion mutation patterns. RT-PCR showed that Ac0 achieved significantly higher exon skipping efficiency in both primary DMD muscle cells compared to analog eteplirsen, or analog drisapersen (FIG. 6A to D): up to 5 and 7 times higher efficiency were observed compared to analog eteplirsen and drisapersen, respectively. A significant efficiency of Ac0-mediated exon 51 skipping was also confirmed Page 34 Header in primary healthy skeletal muscle cells (FIG. 6E and F). Interestingly, with increasing exon 51 skipping efficiency, spontaneous exon 52 skipping, which does not disrupt the reading frame, was observed in primary healthy and DMD muscle cells, and an immortalized DMD muscle cell line with ex48-50 del. (6594).

2.10 In Vivo Efficacy of Ac0 Morpholino and Analog Eteplirsen in hDMD/Dmd-Null Mice

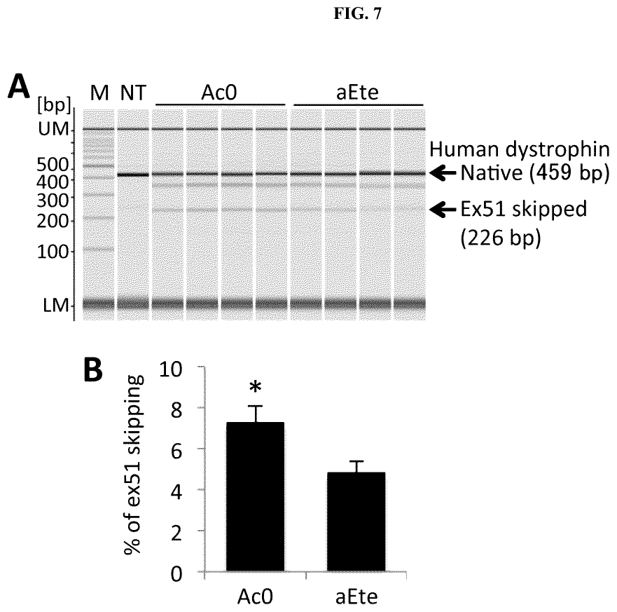

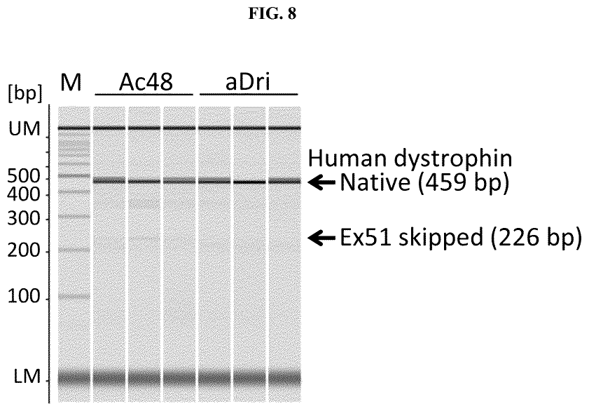

A major hurdle in the development of exon skipping therapy is that human-specific AOs cannot always be tested in an appropriate animal model. This limits the evaluation of in vivo effects of AOs designed for patients. Here, we developed a new mouse model that has the full-length human DMD gene but lacks the entire mouse Dmd gene (hDMD/Dmd-null) to test the in vivo efficacy of human AOs. This mouse model was employed to avoid the cross-reaction between human sequences and mouse sequences (note that conventional mdx mice still have the mouse dystrophin mRNA, which can cross-react with human-targeting AOs), and obtained by cross-breeding between hDMD mice34 and Dmd-null mice35. AcO, Ac48, analog eteplirsen or analog drisapersen was injected into TA muscles of these mice, and the effectiveness of in vivo exon 51 skipping was analysed 2 weeks after the injection. The result showed significantly greater exon skipping efficiency in mice treated with Ac0 compared to analog eteplirsen (FIG. 7). Visible exon 51-skipped bands were found in Ac48-treated mice, with an average exon skipping efficiency of 1.11% (+0.46%, SE). On the other hand, no quantifiable exon 51-skipped bands were observed in mice treated with analog drisapersen (FIG. 8).

SEQUENCES

| | |
|---|---|
| GTGTCACCAGAGTAACAGTCTGAGTAGGAG | Ac0 (SEQ ID NO. 1) |
| AGGTTGTGTCACCAGAGTAACAGTCTGAGT | Ac5 (SEQ ID NO. 2) |
| GGCAGTTTCCTTAGTAACCACAGGTTGTGT | Ac26 (SEQ ID NO. 3) |
| AGATGGCAGTTTCCTTAGTAACCACAGGTT | Ac30 (SEQ ID NO. 4) |
| ATGGCATTTCTAGTTTGGAGATGGCAGTTT | Ac48 (SEQ ID NO. 5) |
| CTCCAACATCAAGGAAGATGGCATTTCTAG | Eteplirsen (SEQ ID NO. 6) |
| TCAAGGAAGATGGCATTTCT | Drisapersen (SEQ ID NO. 7) |
| TGTCACCAGAGTAACAGTCTGAGTAGGAG | hEx51_Ac0-29mer (SEQ ID NO. 8) |
| GTCACCAGAGTAACAGTCTGAGTAGGAG | hEx51_Ac0-28mer (SEQ ID NO. 9) |
| TCACCAGAGTAACAGTCTGAGTAGGAG | hEx51_Ac0-27mer (SEQ ID NO. 10) |
| CACCAGAGTAACAGTCTGAGTAGGAG | hEx51_Ac0-26mer (SEQ ID NO. 11) |
| ACCAGAGTAACAGTCTGAGTAGGAG | hEx51_Ac0-25mer (SEQ ID NO. 12) |
| CCACAGGTTGTGTCACCAGAGTAACAGTCT | Ac9 (SEQ ID NO. 13) |
| TTATAACTTGATCAAGCAGAGAAAGCCAGT | Ac141 (SEQ ID NO. 14) |
| atacCTTCTGCTTGATGATCATCTCGTTGA | Ac207 (SEQ ID NO. 15) |
| CAGCCAGTGAAGAGGAAGTTAG | Ex49/50_94-10_hDMD_Fwd (SEQ ID NO. 16) |
| CCAGCCATTGTGTTGAATCC | Ex53_80-99_hDMD_Rv (SEQ ID NO. 17) |
| AGGACCCGTGCTTGTAAGTG | Ex47_60-79_hDMD_Fwd (SEQ ID NO. 18) |
| GATTGTTCTAGCCTCTTGATTGC | Ex52_83-105_hDMD_Rv (SEQ ID NO. 19) |
| GACAAGGGCGATTTGACAG | Ex43/44_167-12_hDMD_Fwd (SEQ ID NO. 20) |
| CAAGCACTCAGCCAGTGAAG | mDmd_ex49_83-102_Fwd (SEQ ID NO 21) |
| TCCAGCCATTGTGTTGAATC | deletionmDmd_ex53_81-100_Rv (SEQ ID NO. 22) |
| TCCCTGAGCTGAACGGGAAG | hGAPDH_662-81_Fwd (SEQ ID NO. 23) |
| TCCAGCCATTGTGTTGAATC | controlhGAPDH_860-79_Rv (SEQ ID NO. 24) |
| TCGATGCTCTTAGCTGAGTGTCC | h18S_760-82_Fwd (SEQ ID NO. 25) |
| TGATCGTCTTCGAACCTCCG | control h18S_1039-58_Rv (SEQ ID NO. 26) |

TABLE 3

| | | | | | | | | |
|---|---|---|---|---|---|---|---|---|
| 51 | 9 | CCACAGGTTGTGTCACCAGAGTAACAGTCT | 80.49 | 1 | 18 | GTAACCACAGGTTGTGTCACCAGAG | 41.20 | 1 |
| 51 | 0 | GTGTCACCAGAGTAACAGTCTGAGTAGGAG | 80.11 | 2 | 16 | AACCACAGGTTGTGTCACCAGAGTA | 39.94 | 2 |
| 51 | 10 | ACCACAGGTTGTGTCACCAGAGTAACAGTC | 79.98 | 3 | 12 | ACAGGTTGTGTCACCAGAGTAACAG | 38.08 | 3 |
| 51 | 5 | AGGTTGTGTCACCAGAGTAACAGTCTGAGT | 72.97 | 4 | 14 | CCACAGGTTGTGTCACCAGAGTAAC | 37.52 | 4 |
| 51 | 8 | CACAGGTTGTGTCACCAGAGTAACAGTCTG | 72.01 | 5 | 15 | ACCACAGGTTGTGTCACCAGAGTAA | 37.23 | 5 |
| 51 | 1 | TGTGTCACCAGAGTAACAGTCTGAGTAGGA | 71.94 | 6 | 31 | GGCAGTTTCCTTAGTAACCACAGGT | 37.18 | 6 |
| 51 | 2 | TTGTGTCACCAGAGTAACAGTCTGAGTAGG | 71.51 | 7 | 13 | CACAGGTTGTGTCACCAGAGTAACA | 36.66 | 7 |
| 51 | 11 | AACCACAGGTTGTGTCACCAGAGTAACAGT | 70.65 | 8 | 10 | AGGTTGTGTCACCAGAGTAACAGTC | 35.56 | 8 |
| 51 | 6 | CAGGTTGTGTCACCAGAGTAACAGTCTGAG | 68.18 | 9 | 11 | CAGGTTGTGTCACCAGAGTAACAGT | 33.75 | 9 |
| 51 | 7 | ACAGGTTGTGTCACCAGAGTAACAGTCTGA | 68.14 | 10 | 0 | ACCAGAGTAACAGTCTGAGTAGGAG | 33.34 | 10 |
| 51 | 4 | GGTTGTGTCACCAGAGTAACAGTCTGAGTA | 66.65 | 11 | 9 | GGTTGTGTCACCAGAGTAACAGTCT | 33.10 | 11 |
| 51 | 26 | GGCAGTTTCCTTAGTAACCACAGGTTGTGT | 66.32 | 12 | 17 | TAACCACAGGTTGTGTCACCAGAGT | 32.95 | 12 |
| 51 | 18 | CCTTAGTAACCACAGGTTGTGTCACCAGAG | 65.25 | 13 | 32 | TGGCAGTTTCCTTAGTAACCACAGG | 32.77 | 13 |
| 51 | 19 | TCCTTAGTAACCACAGGTTGTGTCACCAGA | 64.81 | 14 | 30 | GCAGTTTCCTTAGTAACCACAGGTT | 31.61 | 14 |
| 51 | 27 | TGGCAGTTTCCTTAGTAACCACAGGTTGTG | 64.09 | 15 | 19 | AGTAACCACAGGTTGTGTCACCAGA | 30.95 | 15 |
| 51 | 12 | TAACCACAGGTTGTGTCACCAGAGTAACAG | 64.08 | 16 | 23 | CCTTAGTAACCACAGGTTGTGTCAC | 30.66 | 16 |
| 51 | 13 | GTAACCACAGGTTGTGTCACCAGAGTAACA | 63.65 | 17 | 5 | GTGTCACCAGAGTAACAGTCTGAGT | 30.54 | 17 |
| 51 | 25 | GCAGTTTCCTTAGTAACCACAGGTTGTGTC | 61.81 | 18 | 1 | CACCAGAGTAACAGTCTGAGTAGGA | 30.26 | 18 |
| 51 | 29 | GATGGCAGTTTCCTTAGTAACCACAGGTTG | 61.44 | 19 | 6 | TGTGTCACCAGAGTAACAGTCTGAG | 29.52 | 19 |
| 51 | 14 | AGTAACCACAGGTTGTGTCACCAGAGTAAC | 57.56 | 20 | 24 | TCCTTAGTAACCACAGGTTGTGTCA | 28.97 | 20 |
| 51 | 23 | AGTTTCCTTAGTAACCACAGGTTGTGTCAC | 57.29 | 21 | 2 | TCACCAGAGTAACAGTCTGAGTAGG | 28.83 | 21 |
| 51 | 3 | GTTGTGTCACCAGAGTAACAGTCTGAGTAG | 56.65 | 22 | 3 | GTCACCAGAGTAACAGTCTGAGTAG | 26.60 | 22 |
| 51 | 17 | CTTAGTAACCACAGGTTGTGTCACCAGAGT | 56.39 | 23 | 25 | TTCCTTAGTAACCACAGGTTGTGTC | 26.23 | 23 |
| 51 | 24 | CAGTTTCCTTAGTAACCACAGGTTGTGTCA | 56.16 | 24 | 27 | GTTTCCTTAGTAACCACAGGTTGTG | 26.11 | 24 |
| 51 | 30 | AGATGGCAGTTTCCTTAGTAACCACAGGTT | 55.46 | 25 | 29 | CAGTTTCCTTAGTAACCACAGGTTG | 24.24 | 25 |
| 51 | 20 | TTCCTTAGTAACCACAGGTTGTGTCACCAG | 53.39 | 26 | 34 | GATGGCAGTTTCCTTAGTAACCACA | 22.08 | 26 |
| 51 | 16 | TTAGTAACCACAGGTTGTGTCACCAGAGTA | 53.04 | 27 | 8 | GTTGTGTCACCAGAGTAACAGTCTG | 21.25 | 27 |
| 51 | 15 | TAGTAACCACAGGTTGTGTCACCAGAGTAA | 52.28 | 28 | 7 | TTGTGTCACCAGAGTAACAGTCTGA | 21.05 | 28 |
| 51 | 22 | GTTTCCTTAGTAACCACAGGTTGTGTCACC | 51.90 | 29 | 4 | TGTCACCAGAGTAACAGTCTGAGTA | 20.02 | 29 |
| 51 | 28 | ATGGCAGTTTCCTTAGTAACCACAGGTTGT | 46.50 | 30 | 26 | TTTCCTTAGTAACCACAGGTTGTGT | 19.62 | 30 |
| 51 | 21 | TTTCCTTAGTAACCACAGGTTGTGTCACCA | 45.73 | 31 | 22 | CTTAGTAACCACAGGTTGTGTCACC | 18.61 | 31 |
| 51 | 31 | GAGATGGCAGTTTCCTTAGTAACCACAGGT | 43.71 | 32 | 33 | ATGGCAGTTTCCTTAGTAACCACAG | 18.23 | 32 |
| 51 | 32 | GGAGATGGCAGTTTCCTTAGTAACCACAGG | 38.58 | 33 | 108 | TCTGTCCAAGCCCGGTTGAAATCTG | 18.06 | 33 |
| 51 | 98 | CCAAGCCCGGTTGAAATCTGCCAGAGCAGG | 36.79 | 34 | 87 | TCTGCCAGAGCAGGTACCTCCAACA | 18.04 | 34 |
| 51 | 77 | CAGAGCAGGTACCTCCAACATCAAGGAAGA | 36.22 | 35 | 98 | CCCGGTTGAAATCTGCCAGAGCAGG | 17.52 | 35 |
| 51 | 46 | GGCATTTCTAGTTTGGAGATGGCAGTTTCC | 35.05 | 36 | 28 | AGTTTCCTTAGTAACCACAGGTTGT | 16.78 | 36 |
| 51 | 102 | CTGTCCAAGCCCGGTTGAAATCTGCCAGAG | 33.96 | 37 | 35 | AGATGGCAGTTTCCTTAGTAACCAC | 16.49 | 37 |
| 51 | 103 | TCTGTCCAAGCCCGGTTGAAATCTGCCAGA | 33.85 | 38 | 20 | TAGTAACCACAGGTTGTGTCACCAG | 16.42 | 38 |
| 51 | 78 | CCAGAGCAGGTACCTCCAACATCAAGGAAG | 32.83 | 39 | 83 | CCAGAGCAGGTACCTCCAACATCAA | 15.89 | 39 |
| 51 | 100 | GTCCAAGCCCGGTTGAAATCTGCCAGAGCA | 32.12 | 40 | 86 | CTGCCAGAGCAGGTACCTCCAACAT | 15.09 | 40 |
| 51 | 101 | TGTCCAAGCCCGGTTGAAATCTGCCAGAGC | 31.85 | 41 | 82 | CAGAGCAGGTACCTCCAACATCAAG | 14.95 | 41 |
| 51 | 53 | GGAAGATGGCATTTCTAGTTTGGAGATGGC | 31.58 | 42 | 84 | GCCAGAGCAGGTACCTCCAACATCA | 14.91 | 42 |
| 51 | 99 | TCCAAGCCCGGTTGAAATCTGCCAGAGCAG | 31.36 | 43 | 85 | TGCCAGAGCAGGTACCTCCAACATC | 14.66 | 43 |
| 51 | 106 | AGTTCTGTCCAAGCCCGGTTGAAATCTGCC | 31.20 | 44 | 110 | GTTCTGTCCAAGCCCGGTTGAAATC | 13.87 | 44 |
| 51 | 33 | TGGAGATGGCAGTTTCCTTAGTAACCACAG | 30.45 | 45 | 107 | CTGTCCAAGCCCGGTTGAAATCTGC | 12.76 | 45 |
| 51 | 105 | GTTCTGTCCAAGCCCGGTTGAAATCTGCCA | 29.76 | 46 | 109 | TTCTGTCCAAGCCCGGTTGAAATCT | 12.71 | 46 |
| 51 | 104 | TTCTGTCCAAGCCCGGTTGAAATCTGCCAG | 29.45 | 47 | 99 | GCCCGGTTGAAATCTGCCAGAGCAG | 12.07 | 47 |
| 51 | 117 | GCCAGTCGGTAAGTTCTGTCCAAGCCCGGT | 28.99 | 48 | 81 | AGAGCAGGTACCTCCAACATCAAGG | 11.09 | 48 |
| 51 | 87 | TGAAATCTGCCAGAGCAGGTACCTCCAACA | 27.92 | 49 | 36 | GAGATGGCAGTTTCCTTAGTAACCA | 10.96 | 49 |
| 51 | 37 | AGTTTGGAGATGGCAGTTTCCTTAGTAACC | 27.37 | 50 | 51 | GGCATTTCTAGTTTGGAGATGGCAG | 10.71 | 50 |
| 51 | 97 | CAAGCCCGGTTGAAATCTGCCAGAGCAGGT | 27.25 | 51 | 111 | AGTTCTGTCCAAGCCCGGTTGAAAT | 10.66 | 51 |
| 51 | 40 | TCTAGTTTGGAGATGGCAGTTTCCTTAGTA | 27.24 | 52 | 21 | TTAGTAACCACAGGTTGTGTCACCA | 10.43 | 52 |
| 51 | 76 | AGAGCAGGTACCTCCAACATCAAGGAAGAT | 27.06 | 53 | 106 | TGTCCAAGCCCGGTTGAAATCTGCC | 10.38 | 53 |
| 51 | 81 | CTGCCAGAGCAGGTACCTCCAACATCAAGG | 26.57 | 54 | 112 | AAGTTCTGTCCAAGCCCGGTTGAAA | 9.48 | 54 |
| 51 | 95 | AGCCCGGTTGAAATCTGCCAGAGCAGGTAC | 26.37 | 55 | 115 | GGTAAGTTCTGTCCAAGCCCGGTTG | 9.33 | 55 |
| 51 | 86 | GAAATCTGCCAGAGCAGGTACCTCCAACAT | 25.98 | 56 | 50 | GCATTTCTAGTTTGGAGATGGCAGT | 9.05 | 56 |
| 51 | 80 | TGCCAGAGCAGGTACCTCCAACATCAAGGA | 25.94 | 57 | 101 | AAGCCCGGTTGAAATCTGCCAGAGC | 8.92 | 57 |
| 51 | 96 | AAGCCCGGTTGAAATCTGCCAGAGCAGGTA | 25.55 | 58 | 103 | CCAAGCCCGGTTGAAATCTGCCAGA | 8.24 | 58 |
| 51 | 79 | GCCAGAGCAGGTACCTCCAACATCAAGGAA | 25.55 | 59 | 113 | TAAGTTCTGTCCAAGCCCGGTTGAA | 7.96 | 59 |
| 51 | 108 | TAAGTTCTGTCCAAGCCCGGTTGAAATCTG | 25.54 | 60 | 105 | GTCCAAGCCCGGTTGAAATCTGCCA | 7.95 | 60 |
| 51 | 90 | GGTTGAAATCTGCCAGAGCAGGTACCTCCA | 25.44 | 61 | 53 | ATGGCATTTCTAGTTTGGAGATGGC | 7.67 | 61 |
| 51 | 50 | AGATGGCATTTCTAGTTTGGAGATGGCAGT | 25.21 | 62 | 100 | AGCCCGGTTGAAATCTGCCAGAGCA | 7.33 | 62 |
| 51 | 89 | GTTGAAATCTGCCAGAGCAGGTACCTCCAA | 24.68 | 63 | 116 | CGGTAAGTTCTGTCCAAGCCCGGTT | 7.17 | 63 |
| 51 | 94 | GCCCGGTTGAAATCTGCCAGAGCAGGTACC | 23.96 | 64 | 97 | CCGGTTGAAATCTGCCAGAGCAGGT | 6.96 | 64 |
| 51 | 88 | TTGAAATCTGCCAGAGCAGGTACCTCCAAC | 23.90 | 65 | 117 | TCGGTAAGTTCTGTCCAAGCCCGGT | 6.90 | 65 |
| 51 | 34 | TTGGAGATGGCAGTTTCCTTAGTAACCACA | 23.77 | 66 | 102 | CAAGCCCGGTTGAAATCTGCCAGAG | 6.69 | 66 |
| 51 | 65 | CTCCAACATCAAGGAAGATGGCATTTCTAG | 23.66 | 67 | 114 | GTAAGTTCTGTCCAAGCCCGGTTGA | 6.65 | 67 |
| 51 | 35 | TTTGGAGATGGCAGTTTCCTTAGTAACCAC | 23.53 | 68 | 104 | TCCAAGCCCGGTTGAAATCTGCCAG | 6.63 | 68 |
| 51 | 91 | CGGTTGAAATCTGCCAGAGCAGGTACCTCC | 23.52 | 69 | 91 | GAAATCTGCCAGAGCAGGTACCTCC | 6.24 | 69 |
| 51 | 45 | GCATTTCTAGTTTGGAGATGGCAGTTTCCT | 23.40 | 70 | 37 | GGAGATGGCAGTTTCCTTAGTAACC | 6.17 | 70 |
| 51 | 57 | TCAAGGAAGATGGCATTTCTAGTTTGGAGA | 23.34 | 71 | 88 | ATCTGCCAGAGCAGGTACCTCCAAC | 6.14 | 71 |
| 51 | 118 | AGCCAGTCGGTAAGTTCTGTCCAAGCCCGG | 23.29 | 72 | 118 | GTCGGTAAGTTCTGTCCAAGCCCGG | 5.97 | 72 |
| 51 | 82 | TCTGCCAGAGCAGGTACCTCCAACATCAAG | 22.86 | 73 | 95 | GGTTGAAATCTGCCAGAGCAGGTAC | 5.25 | 73 |
| 51 | 93 | CCCGGTTGAAATCTGCCAGAGCAGGTACCT | 22.81 | 74 | 57 | GAAGATGGCATTTCTAGTTTGGAGA | 5.02 | 74 |
| 51 | 107 | AAGTTCTGTCCAAGCCCGGTTGAAATCTGC | 22.77 | 75 | 46 | TTCTAGTTTGGAGATGGCAGTTTCC | 5.00 | 75 |
| 51 | 116 | CCAGTCGGTAAGTTCTGTCCAAGCCCGGTT | 22.52 | 76 | 47 | TTTCTAGTTTGGAGATGGCAGTTTC | 4.87 | 76 |
| 51 | 109 | GTAAGTTCTGTCCAAGCCCGGTTGAAATCT | 22.51 | 77 | 89 | AATCTGCCAGAGCAGGTACCTCCAA | 4.60 | 77 |
| 51 | 110 | GGTAAGTTCTGTCCAAGCCCGGTTGAAATC | 22.46 | 78 | 58 | GGAAGATGGCATTTCTAGTTTGGAG | 4.30 | 78 |
| 51 | 92 | CCGGTTGAAATCTGCCAGAGCAGGTACCTC | 22.07 | 79 | 96 | CGGTTGAAATCTGCCAGAGCAGGTA | 3.79 | 79 |
| 51 | 36 | GTTTGGAGATGGCAGTTTCCTTAGTAACCA | 21.97 | 80 | 131 | GCAGAGAAAGCCAGTCGGTAAGTTC | 3.63 | 80 |
| 51 | 56 | CAAGGAAGATGGCATTTCTAGTTTGGAGAT | 21.85 | 81 | 80 | GAGCAGGTACCTCCAACATCAAGGA | 3.51 | 81 |
| 51 | 75 | GAGCAGGTACCTCCAACATCAAGGAAGATG | 21.81 | 82 | 120 | CAGTCGGTAAGTTCTGTCCAAGCCC | 3.35 | 82 |
| 51 | 39 | CTAGTTTGGAGATGGCAGTTTCCTTAGTAA | 21.62 | 83 | 128 | GAGAAAGCCAGTCGGTAAGTTCTGT | 3.12 | 83 |
| 51 | 64 | TCCAACATCAAGGAAGATGGCATTTCTAGT | 21.18 | 84 | 119 | AGTCGGTAAGTTCTGTCCAAGCCCG | 3.09 | 84 |
| 51 | 115 | CAGTCGGTAAGTTCTGTCCAAGCCCGGTTG | 20.90 | 85 | 92 | TGAAATCTGCCAGAGCAGGTACCTC | 2.10 | 85 |
| 51 | 52 | GAAGATGGCATTTCTAGTTTGGAGATGGCA | 20.77 | 86 | 40 | TTTGGAGATGGCAGTTTCCTTAGTA | 1.87 | 86 |
| 51 | 63 | CCAACATCAAGGAAGATGGCATTTCTAGTT | 20.61 | 87 | 90 | AAATCTGCCAGAGCAGGTACCTCCA | 1.74 | 87 |
| 51 | 111 | CGGTAAGTTCTGTCCAAGCCCGGTTGAAAT | 20.60 | 88 | 129 | AGAGAAAGCCAGTCGGTAAGTTCTG | 1.57 | 88 |
| 51 | 41 | TTCTAGTTTGGAGATGGCAGTTTCCTTAGT | 20.19 | 89 | 121 | CCAGTCGGTAAGTTCTGTCCAAGCC | 1.52 | 89 |
| 51 | 84 | AATCTGCCAGAGCAGGTACCTCCAACATCA | 19.86 | 90 | 122 | GCCAGTCGGTAAGTTCTGTCCAAGC | 0.99 | 90 |
| 51 | 113 | GTCGGTAAGTTCTGTCCAAGCCCGGTTGAA | 19.80 | 91 | 38 | TGGAGATGGCAGTITCCITAGTAAC | 0.44 | 91 |
| 51 | 114 | AGTCGGTAAGTTCTGTCCAAGCCCGGTTGA | 19.75 | 92 | 45 | TCTAGTTTGGAGATGGCAGTTTCCT | 0.40 | 92 |
| 51 | 73 | GCAGGTACCTCCAACATCAAGGAAGATGGC | 19.30 | 93 | 78 | GCAGGTACCTCCAACATCAAGGAAG | 0.10 | 93 |
| 51 | 38 | TAGTTTGGAGATGGCAGTTTCCTTAGTAAC | 19.21 | 94 | 48 | ATTTCTAGTTTGGAGATGGCAGTTT | −0.21 | 94 |
| 51 | 119 | AAGCCAGTCGGTAAGTTCTGTCCAAGCCCG | 19.04 | 95 | 125 | AAAGCCAGTCGGTAAGTTCTGTCCA | −0.23 | 95 |
| 51 | 67 | ACCTCCAACATCAAGGAAGATGGCATTTCT | 19.03 | 96 | 126 | GAAAGCCAGTCGGTAAGTTCTGTCC | −0.58 | 96 |
| 51 | 83 | ATCTGCCAGAGCAGGTACCTCCAACATCAA | 18.98 | 97 | 39 | TTGGAGATGGCAGTTTCCTTAGTAA | −0.64 | 97 |
| 51 | 58 | ATCAAGGAAGATGGCATTTCTAGTTTGGAG | 18.74 | 98 | 134 | CAAGCAGAGAAAGCCAGTCGGTAAG | −0.80 | 98 |
| 51 | 112 | TCGGTAAGTTCTGTCCAAGCCCGGTTGAAA | 18.59 | 99 | 132 | AGCAGAGAAAGCCAGTCGGTAAGTT | −0.92 | 99 |
| 51 | 54 | AGGAAGATGGCATTTCTAGTTTGGAGATGG | 18.20 | 100 | 137 | GATCAAGCAGAGAAAGCCAGTCGGT | −0.94 | 100 |
| 51 | 85 | AAATCTGCCAGAGCAGGTACCTCCAACATC | 17.93 | 101 | 133 | AAGCAGAGAAAGCCAGTCGGTAAGT | −1.00 | 101 |
| 51 | 66 | CCTCCAACATCAAGGAAGATGGCATTTCTA | 17.65 | 102 | 138 | TGATCAAGCAGAGAAAGCCAGTCGG | −1.03 | 102 |
| 51 | 49 | GATGGCATTTCTAGTTTGGAGATGGCAGTT | 17.50 | 103 | 127 | AGAAAGCCAGTCGGTAAGTTCTGTC | −1.07 | 103 |
| 51 | 42 | TTTCTAGTTTGGAGATGGCAGTTTCCTTAG | 17.40 | 104 | 136 | ATCAAGCAGAGAAAGCCAGTCGGTA | −1.36 | 104 |
| 51 | 44 | CATTTCTAGTTTGGAGATGGCAGTTTCCTT | 16.99 | 105 | 72 | ACCTCCAACATCAAGGAAGATGGCA | −1.50 | 105 |
| 51 | 51 | AAGATGGCATTTCTAGTTTGGAGATGGCAG | 16.77 | 106 | 79 | AGCAGGTACCTCCAACATCAAGGAA | −1.50 | 106 |
| 51 | 55 | AAGGAAGATGGCATTTCTAGTTTGGAGATG | 16.50 | 107 | 130 | CAGAGAAAGCCAGTCGGTAAGTTCT | −1.79 | 107 |
| 51 | 68 | TACCTCCAACATCAAGGAAGATGGCATTTC | 16.36 | 108 | 94 | GTTGAAATCTGCCAGAGCAGGTACC | −1.88 | 108 |
| 51 | 121 | GAAAGCCAGTCGGTAAGTTCTGTCCAAGCC | 16.27 | 109 | 59 | AGGAAGATGGCATTTCTAGTTTGGA | −2.14 | 109 |
| 51 | 120 | AAAGCCAGTCGGTAAGTTCTGTCCAAGCCC | 16.02 | 110 | 56 | AAGATGGCATTTCTAGTTTGGAGAT | −2.16 | 110 |
| 51 | 74 | AGCAGGTACCTCCAACATCAAGGAAGATGG | 15.45 | 111 | 93 | TTGAAATCTGCCAGAGCAGGTACCT | −2.19 | 111 |
| 51 | 122 | AGAAAGCCAGTCGGTAAGTTCTGTCCAAGC | 15.40 | 112 | 140 | CTTGATCAAGCAGAGAAAGCCAGTC | −2.58 | 112 |
| 51 | 59 | CATCAAGGAAGATGGCATTTCTAGTTTGGA | 15.35 | 113 | 70 | CTCCAACATCAAGGAAGATGGCATT | −2.88 | 113 |
| 51 | 123 | GAGAAAGCCAGTCGGTAAGTTCTGTCCAAG | 15.01 | 114 | 71 | CCTCCAACATCAAGGAAGATGGCAT | −2.97 | 114 |
| 51 | 69 | GTACCTCCAACATCAAGGAAGATGGCATTT | 14.79 | 115 | 52 | TGGCATTTCTAGTTTGGAGATGGCA | −3.00 | 115 |
| 51 | 60 | ACATCAAGGAAGATGGCATTTCTAGTTTGG | 14.56 | 116 | 63 | ATCAAGGAAGATGGCATTTCTAGTT | −3.06 | 116 |
| 51 | 72 | CAGGTACCTCCAACATCAAGGAAGATGGCA | 13.59 | 117 | 42 | AGTTTGGAGATGGCAGTTTCCTTAG | −3.16 | 117 |
| 51 | 124 | AGAGAAAGCCAGTCGGTAAGTTCTGTCCAA | 12.92 | 118 | 54 | GATGGCATTTCTAGTTTGGAGATGG | −3.16 | 118 |
| 51 | 70 | GGTACCTCCAACATCAAGGAAGATGGCATT | 12.45 | 119 | 49 | CATTTCTAGTTTGGAGATGGCAGTT | −3.37 | 119 |
| 51 | 71 | AGGTACCTCCAACATCAAGGAAGATGGCAT | 12.24 | 120 | 135 | TCAAGCAGAGAAAGCCAGTCGGTAA | −3.39 | 120 |
| 51 | 62 | CAACATCAAGGAAGATGGCATTTCTAGTTT | 12.18 | 121 | 123 | AGCCAGTCGGTAAGTTCTGTCCAAG | −3.44 | 121 |
| 51 | 61 | AACATCAAGGAAGATGGCATTTCTAGTTTG | 12.09 | 122 | 141 | ACTTGATCAAGCAGAGAAAGCCAGT | −3.99 | 122 |
| 51 | 126 | GCAGAGAAAGCCAGTCGGTAAGTTCTGTCC | 12.04 | 123 | 68 | CCAACATCAAGGAAGATGGCATTTC | −4.03 | 123 |
| 51 | 125 | CAGAGAAAGCCAGTCGGTAAGTTCTGTCCA | 11.49 | 124 | 124 | AAGCCAGTCGGTAAGTTCTGTCCAA | −4.20 | 124 |
| 51 | 43 | ATTTCTAGTTTGGAGATGGCAGTTTCCTTA | 11.13 | 125 | 60 | AAGGAAGATGGCATTTCTAGTTTGG | −4.22 | 125 |
| 51 | 47 | TGGCATTTCTAGTTTGGAGATGGCAGTTTC | 11.09 | 126 | 55 | AGATGGCATTTCTAGTTTGGAGATG | −4.42 | 126 |
| 51 | 129 | CAAGCAGAGAAAGCCAGTCGGTAAGTTCTG | 10.80 | 127 | 142 | AACTTGATCAAGCAGAGAAAGCCAG | −4.53 | 127 |
| 51 | 48 | ATGGCATTTCTAGTTTGGAGATGGCAGTTT | 10.61 | 128 | 73 | TACCTCCAACATCAAGGAAGATGGC | −4.70 | 128 |
| 51 | 130 | TCAAGCAGAGAAAGCCAGTCGGTAAGTTCT | 10.43 | 129 | 139 | TTGATCAAGCAGAGAAAGCCAGTCG | −4.97 | 129 |
| 51 | 128 | AAGCAGAGAAAGCCAGTCGGTAAGTTCTGT | 9.84 | 130 | 41 | GTTTGGAGATGGCAGTTTCCTTAGT | −5.02 | 130 |
| 51 | 131 | ATCAAGCAGAGAAAGCCAGTCGGTAAGTTC | 8.53 | 131 | 74 | GTACCTCCAACATCAAGGAAGATGG | −5.30 | 131 |
| 51 | 127 | AGCAGAGAAAGCCAGTCGGTAAGTTCTGTC | 8.26 | 132 | 64 | CATCAAGGAAGATGGCATTTCTAGT | −5.36 | 132 |
| 51 | 136 | ACTTGATCAAGCAGAGAAAGCCAGTCGGTA | 6.43 | 133 | 65 | ACATCAAGGAAGATGGCATTTCTAG | −5.43 | 133 |
| 51 | 137 | AACTTGATCAAGCAGAGAAAGCCAGTCGGT | 5.84 | 134 | 44 | CTAGTTTGGAGATGGCAGTTTCCTT | −6.16 | 134 |
| 51 | 138 | TAACTTGATCAAGCAGAGAAAGCCAGTCGG | 5.26 | 135 | 61 | CAAGGAAGATGGCATTTCTAGTTTG | −6.27 | 135 |
| 51 | 132 | GATCAAGCAGAGAAAGCCAGTCGGTAAGTT | 4.60 | 136 | 172 | GTCACCCACCATCACCCTCTGTGAT | −7.68 | 136 |
| 51 | 140 | TATAACTTGATCAAGCAGAGAAAGCCAGTC | 4.36 | 137 | 69 | TCCAACATCAAGGAAGATGGCATTT | −7.84 | 137 |
| 51 | 133 | TGATCAAGCAGAGAAAGCCAGTCGGTAAGT | 4.31 | 138 | 143 | TAACTTGATCAAGCAGAGAAAGCCA | −8.29 | 138 |
| 51 | 134 | TTGATCAAGCAGAGAAAGCCAGTCGGTAAG | 3.75 | 139 | 62 | TCAAGGAAGATGGCATTTCTAGTTT | −8.30 | 139 |
| 51 | 139 | ATAACTTGATCAAGCAGAGAAAGCCAGTCG | 2.70 | 140 | 77 | CAGGTACCTCCAACATCAAGGAAGA | −8.53 | 140 |
| 51 | 135 | CTTGATCAAGCAGAGAAAGCCAGTCGGTAA | 2.28 | 141 | 67 | CAACATCAAGGAAGATGGCATTTCT | −8.81 | 141 |
| 51 | 141 | TTATAACTTGATCAAGCAGAGAAAGCCAGT | 1.76 | 142 | 173 | GGTCACCCACCATCACCCTCTGTGA | −8.87 | 142 |
| 51 | 142 | TTTATAACTTGATCAAGCAGAGAAAGCCAG | −0.17 | 143 | 144 | ATAACTTGATCAAGCAGAGAAAGCC | −8.98 | 143 |
| 51 | 146 | TGATTTTATAACTTGATCAAGCAGAGAAAG | −3.06 | 144 | 75 | GGTACCTCCAACATCAAGGAAGATG | −9.18 | 144 |
| 51 | 145 | GATTTTATAACTTGATCAAGCAGAGAAAGC | −3.17 | 145 | 174 | AGGTCACCCACCATCACCCTCTGTG | −9.52 | 145 |
| 51 | 156 | TCACCCTCTGTGATTTTATAACTTGATCAA | −3.49 | 146 | 66 | AACATCAAGGAAGATGGCATTTCTA | −9.58 | 146 |
| 51 | 167 | GTCACCCACCATCACCCTCTGTGATTTTAT | −3.97 | 147 | 43 | TAGTTTGGAGATGGCAGTTTCCTTA | −9.61 | 147 |
| 51 | 143 | TTTTATAACTTGATCAAGCAGAGAAAGCCA | −4.01 | 148 | 146 | TTATAACTTGATCAAGCAGAGAAAG | −9.74 | 148 |
| 51 | 174 | CCTCAAGGTCACCCACCATCACCCTCTGTG | −4.37 | 149 | 171 | TCACCCACCATCACCCTCTGTGATT | −10.00 | 149 |
| 51 | 144 | ATTTTATAACTTGATCAAGCAGAGAAAGCC | −4.66 | 150 | 170 | CACCCACCATCACCCTCTGTGATTT | −10.19 | 150 |
| 51 | 155 | CACCCTCTGTGATTTTATAACTTGATCAAG | −5.42 | 151 | 147 | TTTATAACTTGATCAAGCAGAGAAA | −10.27 | 151 |
| 51 | 168 | GGTCACCCACCATCACCCTCTGTGATTTTA | −5.53 | 152 | 176 | CAAGGTCACCCACCATCACCCTCTG | −11.06 | 152 |
| 51 | 175 | TCCTCAAGGTCACCCACCATCACCCTCTGT | −5.64 | 153 | 145 | TATAACTTGATCAAGCAGAGAAAGC | −11.20 | 153 |
| 51 | 157 | ATCACCCTCTGTGATTTTATAACTTGATCA | −5.75 | 154 | 168 | CCCACCATCACCCTCTGTGATTTTA | −11.23 | 154 |
| 51 | 173 | CTCAAGGTCACCCACCATCACCCTCTGTGA | −6.14 | 155 | 169 | ACCCACCATCACCCTCTGTGATTTT | −11.29 | 155 |
| 51 | 176 | ATCCTCAAGGTCACCCACCATCACCCTCTG | −6.36 | 156 | 177 | TCAAGGTCACCCACCATCACCCTCT | −11.45 | 156 |
| 51 | 172 | TCAAGGTCACCCACCATCACCCTCTGTGAT | −6.44 | 157 | 181 | ATCCTCAAGGTCACCCACCATCACC | −11.50 | 157 |
| 51 | 166 | TCACCCACCATCACCCTCTGTGATTTTATA | −6.45 | 158 | 175 | AAGGTCACCCACCATCACCCTCTGT | −11.55 | 158 |
| 51 | 163 | CCCACCATCACCCTCTGTGATTTTATAACT | −6.75 | 159 | 180 | TCCTCAAGGTCACCCACCATCACCC | −11.59 | 159 |
| 51 | 161 | CACCATCACCCTCTGTGATTTTATAACTTG | −6.93 | 160 | 179 | CCTCAAGGTCACCCACCATCACCCT | −11.76 | 160 |
| 51 | 147 | GTGATTTTATAACTTGATCAAGCAGAGAAA | −7.04 | 161 | 183 | ATATCCTCAAGGTCACCCACCATCA | −11.76 | 161 |
| 51 | 164 | ACCCACCATCACCCTCTGTGATTTTATAAC | −7.04 | 162 | 178 | CTCAAGGTCACCCACCATCACCCTC | −11.79 | 162 |
| 51 | 171 | CAAGGTCACCCACCATCACCCTCTGTGATT | −7.07 | 163 | 76 | AGGTACCTCCAACATCAAGGAAGAT | −12.00 | 163 |
| 51 | 169 | AGGTCACCCACCATCACCCTCTGTGATTTT | −7.08 | 164 | 182 | TATCCTCAAGGTCACCCACCATCAC | −12.19 | 164 |
| 51 | 153 | CCCTCTGTGATTTTATAACTTGATCAAGCA | −7.09 | 165 | 156 | CTCTGTGATTTTATAACTTGATCAA | −12.23 | 165 |
| 51 | 165 | CACCCACCATCACCCTCTGTGATTTTATAA | −7.12 | 166 | 167 | CCACCATCACCCTCTGTGATTTTAT | −12.52 | 166 |
| 51 | 178 | ATATCCTCAAGGTCACCCACCATCACCCTC | −7.12 | 167 | 157 | CCTCTGTGATTTTATAACTTGATCA | −12.99 | 167 |
| 51 | 160 | ACCATCACCCTCTGTGATTTTATAACTTGA | −7.13 | 168 | 184 | GATATCCTCAAGGTCACCCACCATC | −13.02 | 168 |
| 51 | 177 | TATCCTCAAGGTCACCCACCATCACCCTCT | −7.18 | 169 | 148 | TTTTATAACTTGATCAAGCAGAGAA | −13.20 | 169 |
| 51 | 154 | ACCCTCTGTGATTTTATAACTTGATCAAGC | −7.53 | 170 | 190 | CTCGTTGATATCCTCAAGGTCACCC | −13.32 | 170 |
| 51 | 170 | AAGGTCACCCACCATCACCCTCTGTGATTT | −7.89 | 171 | 189 | TCGTTGATATCCTCAAGGTCACCCA | −13.68 | 171 |
| 51 | 162 | CCACCATCACCCTCTGTGATTTTATAACTT | −8.37 | 172 | 185 | TGATATCCTCAAGGTCACCCACCAT | −13.68 | 172 |
| 51 | 159 | CCATCACCCTCTGTGATTTTATAACTTGAT | −8.43 | 173 | 188 | CGTTGATATCCTCAAGGTCACCCAC | −13.80 | 173 |
| 51 | 158 | CATCACCCTCTGTGATTTTATAACTTGATC | −8.95 | 174 | 149 | ATTTTATAACTTGATCAAGCAGAGA | −13.86 | 174 |
| 51 | 179 | GATATCCTCAAGGTCACCCACCATCACCCT | −9.14 | 175 | 191 | TCTCGTTGATATCCTCAAGGTCACC | −13.88 | 175 |
| 51 | 152 | CCTCTGTGATTTTATAACTTGATCAAGCAG | −9.35 | 176 | 160 | CACCCTCTGTGATTTTATAACTTGA | −13.94 | 176 |
| 51 | 149 | CTGTGATTTTATAACTTGATCAAGCAGAGA | −9.51 | 177 | 187 | GTTGATATCCTCAAGGTCACCCACC | −14.07 | 177 |
| 51 | 180 | TGATATCCTCAAGGTCACCCACCATCACCC | −9.82 | 178 | 155 | TCTGTGATTTTATAACTTGATCAAG | −14.17 | 178 |
| 51 | 181 | TTGATATCCTCAAGGTCACCCACCATCACC | −9.89 | 179 | 158 | CCCTCTGTGATTTTATAACTTGATC | −14.23 | 179 |
| 51 | 148 | TGTGATTTTATAACTTGATCAAGCAGAGAA | −10.04 | 180 | 192 | ATCTCGTTGATATCCTCAAGGTCAC | −14.36 | 180 |
| 51 | 182 | GTTGATATCCTCAAGGTCACCCACCATCAC | −10.09 | 181 | 186 | TTGATATCCTCAAGGTCACCCACCA | −14.42 | 181 |
| 51 | 183 | CGTTGATATCCTCAAGGTCACCCACCATCA | −10.50 | 182 | 193 | CATCTCGTTGATATCCTCAAGGTCA | −14.55 | 182 |
| 51 | 184 | TCGTTGATATCCTCAAGGTCACCCACCATC | −10.67 | 183 | 161 | TCACCCTCTGTGATTTTATAACTTG | −14.60 | 183 |
| 51 | 185 | CTCGTTGATATCCTCAAGGTCACCCACCAT | −10.79 | 184 | 166 | CACCATCACCCTCTGTGATITTATA | −14.64 | 184 |
| 51 | 186 | TCTCGTTGATATCCTCAAGGTCACCCACCA | −11.32 | 185 | 194 | TCATCTCGTTGATATCCTCAAGGTC | −14.75 | 185 |
| 51 | 188 | CATCTCGTTGATATCCTCAAGGTCACCCAC | −11.44 | 186 | 196 | GATCATCTCGTTGATATCCTCAAGG | −14.84 | 186 |
| 51 | 189 | TCATCTCGTTGATATCCTCAAGGTCACCCA | −11.81 | 187 | 150 | GATTTTATAACTTGATCAAGCAGAG | −14.89 | 187 |
| 51 | 187 | ATCTCGTTGATATCCTCAAGGTCACCCACC | −11.82 | 188 | 197 | TGATCATCTCGTTGATATCCTCAAG | −14.91 | 188 |
| 51 | 190 | ATCATCTCGTTGATATCCTCAAGGTCACCC | −12.10 | 189 | 199 | GATGATCATCTCGTTGATATCCTCA | −14.93 | 189 |
| 51 | 191 | GATCATCTCGTTGATATCCTCAAGGTCACC | −12.59 | 190 | 198 | ATGATCATCTCGTTGATATCCTCAA | −14.94 | 190 |
| 51 | 151 | CTCTGTGATTTTATAACTTGATCAAGCAGA | −12.71 | 191 | 200 | TGATGATCATCTCGTTGATATCCTC | −14.98 | 191 |
| 51 | 192 | TGATCATCTCGTTGATATCCTCAAGGTCAC | −13.26 | 192 | 201 | TTGATGATCATCTCGTTGATATCCT | −15.00 | 192 |
| 51 | 150 | TCTGTGATTTTATAACTTGATCAAGCAGAG | −13.47 | 193 | 195 | ATCATCTCGTTGATATCCTCAAGGT | −15.11 | 193 |
| 51 | 193 | ATGATCATCTCGTTGATATCCTCAAGGTCA | −13.85 | 194 | 202 | CTTGATGATCATCTCGTTGATATCC | −15.16 | 194 |
| 51 | 194 | GATGATCATCTCGTTGATATCCTCAAGGTC | −13.95 | 195 | 159 | ACCCTCTGTGATTTTATAACTTGAT | −15.57 | 195 |
| 51 | 195 | TGATGATCATCTCGTTGATATCCTCAAGGT | −14.35 | 196 | 204 | TGCTTGATGATCATCrCGTTGATAT | −15.61 | 196 |
| 51 | 196 | TTGATGATCATCTCGTTGATATCCTCAAGG | −14.51 | 197 | 203 | GCTTGATGATCATCTCGTTGATATC | −15.64 | 197 |
| 51 | 198 | GCTTGATGATCATCTCGTTGATATCCTCAA | −14.58 | 198 | 164 | CCATCACCCrCTGTGATTTTATAAC | −15.79 | 198 |
| 51 | 197 | CTTGATGATCATCTCGTTGATATCCTCAAG | −14.70 | 199 | 205 | CTGCTTGATGATCATCTCGTTGATA | −16.24 | 199 |
| 51 | 199 | TGCTTGATGATCATCTCGTTGATATCCTCA | −14.77 | 200 | 165 | ACCATCACCCTCTGTGATTTTATAA | −16.41 | 200 |
| 51 | 200 | CTGCTTGATGATCATCTCGTTGATATCCTC | −15.02 | 201 | 163 | CATCACCCTCTGTGATTTTATAACT | −16.49 | 201 |
| 51 | 201 | TCTGCTTGATGATCATCTCGTTGATATCCT | −15.29 | 202 | 206 | TCTGCTTGATGATCATCTCGTTGAT | −16.57 | 202 |
| 51 | 202 | TTCTGCTTGATGATCATCTCGTTGATATCC | −15.67 | 203 | 207 | TTCTGCTTGATGATCATCTCGTTGA | −16.92 | 203 |
| 51 | 203 | CTTCTGCTTGATGATCATCTCGTTGATATC | −16.24 | 204 | 208 | CTTCTGCTTGATGATCATCTCGTTG | −17.32 | 204 |
| 51 | | | | | 154 | CTGTGATTTTATAACTTGATCAAGC | −17.42 | 205 |
| 51 | | | | | 162 | ATCACCCTCTGTGATTTTATAACTT | −17.53 | 206 |
| 51 | | | | | 152 | GTGATTTTATAACTTGATCAAGCAG | −17.91 | 207 |
| 51 | | | | | 151 | TGATTTTATAACTTGATCAAGCAGA | −17.98 | 208 |
| 51 | | | | | 153 | TGTGATTTTATAACTTGATCAAGCA | −19.57 | 209 |

What is claimed is:

1. A method of increasing human dystrophin protein expression in a cell comprising:
contacting the cell with a conjugate comprising a morpholino antisense oligonucleotide (PMO) consisting of SEQ ID NO:1 (Ac0), SEQ ID NO:2 (Ac5), SEQ ID NO:3 (Ac26), SEQ ID NO:4 (Ac30), or SEQ ID NO:5 (Ac48), wherein the PMO binds to exon 51 of human dystrophin pre-mRNA within the region between 0 and +89 of the pre-mRNA sequence.

2. The method of claim 1, wherein the conjugate further comprises a carrier, wherein the carrier is conjugated to the PMO.

3. The method of claim 2, wherein the carrier is operable to transport the PMO into a target cell.

4. The method of claim 2, wherein the carrier is selected from a peptide, a small molecule chemical, a polymer, a nanoparticle, a lipid, a liposome or an exosome.

5. The method of claim 2, wherein the carrier is a cell penetrating peptide.

6. The method of claim 2, wherein the carrier is an arginine-rich cell penetrating peptide.

7. The method of claim 1, wherein the PMO binds to exon 51 of human dystrophin pre-mRNA within the region between 0 and +88, 0 and +87, 0 and +86, 0 and +85, 0 and +84, 0 and +83, 0 and +82, 0 and +81, 0 and +80, 0 and +79, or 0 and +78 of the pre-mRNA sequence.

8. The method of claim 1, wherein the PMO binds to exon 51 of human dystrophin pre-mRNA within the region between 0 and +78 of the pre-mRNA sequence.

9. The method of claim 1, wherein the PMO is at least 70%, at least 80%, at least 90%, at least 95%, at least 96%, at least 97%, at least 98% at least 99% complementary to a sequence of exon 51 of human dystrophin pre-mRNA falling within the region.

10. The method of claim 1, wherein the PMO is hybridisable to a sequence of exon 51 of human dystrophin pre-mRNA falling within the region.

11. A pharmaceutical composition comprising: a conjugate comprising a morpholino antisense oligonucleotide (PMO) consisting of SEQ ID NO:1 (Ac0), SEQ ID NO:2 (Ac5), SEQ ID NO:3 (Ac26), SEQ ID NO:4 (Ac30), or SEQ ID NO:5 (Ac48); and a pharmaceutically acceptable excipient.

12. The pharmaceutical composition of claim 11, wherein the conjugate further comprises a carrier, wherein the carrier is conjugated to the PMO.

13. The pharmaceutical composition of claim 12, wherein the carrier is operable to transport the PMO into a target cell.

14. The pharmaceutical composition of claim 12, wherein the carrier is selected from a peptide, a small molecule chemical, a polymer, a nanoparticle, a lipid, a liposome or an exosome.

15. The pharmaceutical composition of claim 12, wherein the carrier is a cell penetrating peptide.

16. The pharmaceutical composition of claim 12, wherein the carrier is an arginine-rich cell penetrating peptide.

17. The pharmaceutical composition of claim 11, wherein the PMO binds to exon 51 of human dystrophin pre-mRNA within the region between 0 and +88, 0 and +87, 0 and +86, 0 and +85, 0 and +84, 0 and +83, 0 and +82, 0 and +81, 0 and +80, 0 and +79, or 0 and +78 of the pre-mRNA sequence.

18. The pharmaceutical composition of claim 11, wherein the PMO binds to exon 51 of human dystrophin pre-mRNA within the region between 0 and +78 of the pre-mRNA sequence.

19. The pharmaceutical composition of claim 11, wherein the PMO is at least 70%, at least 80%, at least 90%, at least 95%, at least 96%, at least 97%, at least 98% at least 99% complementary to a sequence of exon 51 of human dystrophin pre-mRNA falling within the region.

20. The pharmaceutical composition of claim 11, wherein the PMO is hybridisable to a sequence of exon 51 of human dystrophin pre-mRNA falling within the region.


SEQUENCE LISTING (440 sequences)

SEQ ID NO: 1 - DNA, 30 bp, synthetic construct
  misc_feature 1..30: note = morpholino antisense oligonucleotide
  source 1..30: mol_type = other DNA
gtgtcaccagagtaacagtctgagtaggag

SEQ ID NO: 2 - DNA, 30 bp, synthetic construct
  misc_feature 1..30: note = morpholino antisense oligonucleotide
  source 1..30: mol_type = other DNA
aggttgtgtcaccagagtaacagtctgagt

SEQ ID NO: 3 - DNA, 30 bp, synthetic construct
  misc_feature 1..30: note = morpholino antisense oligonucleotide
  source 1..30: mol_type = other DNA
ggcagtttccttagtaaccacaggttgtgt

SEQ ID NO: 4 - DNA, 30 bp, synthetic construct
  misc_feature 1..30: note = morpholino antisense oligonucleotide
  source 1..30: mol_type = other DNA
agatggcagtttccttagtaaccacaggtt

SEQ ID NO: 5 - DNA, 30 bp, synthetic construct
  misc_feature 1..30: note = morpholino antisense oligonucleotide
  source 1..30: mol_type = other DNA
atggcatttctagtttggagatggcagttt

SEQ ID NO: 6 - DNA, 30 bp, synthetic construct
  misc_feature 1..30: note = morpholino antisense oligonucleotide
  source 1..30: mol_type = other DNA
ctccaacatcaaggaagatggcatttctag

SEQ ID NO: 7 - DNA, 20 bp, synthetic construct
  misc_feature 1..20: note = morpholino antisense oligonucleotide
  source 1..20: mol_type = other DNA
tcaaggaagatggcatttct

SEQ ID NO: 8 - DNA, 29 bp, synthetic construct
  misc_feature 1..29: note = morpholino antisense oligonucleotide
  source 1..29: mol_type = other DNA
tgtcaccagagtaacagtctgagtaggag

SEQ ID NO: 9 - DNA, 28 bp, synthetic construct
  misc_feature 1..28: note = morpholino antisense oligonucleotide
  source 1..28: mol_type = other DNA
gtcaccagagtaacagtctgagtaggag

SEQ ID NO: 10 - DNA, 27 bp, synthetic construct
  misc_feature 1..27: note = morpholino antisense oligonucleotide
  source 1..27: mol_type = other DNA
tcaccagagtaacagtctgagtaggag

SEQ ID NO: 11 - DNA, 26 bp, synthetic construct
  misc_feature 1..26: note = morpholino antisense oligonucleotide
  source 1..26: mol_type = other DNA
caccagagtaacagtctgagtaggag

SEQ ID NO: 12 - DNA, 25 bp, synthetic construct
  misc_feature 1..25: note = morpholino antisense oligonucleotide
  source 1..25: mol_type = other DNA
accagagtaacagtctgagtaggag

SEQ ID NO: 13 - DNA, 30 bp, synthetic construct
  misc_feature 1..30: note = morpholino antisense oligonucleotide
  source 1..30: mol_type = other DNA
ccacaggttgtgtcaccagagtaacagtct

SEQ ID NO: 14 - DNA, 30 bp, synthetic construct
  misc_feature 1..30: note = morpholino antisense oligonucleotide
  source 1..30: mol_type = other DNA
ttataacttgatcaagcagagaaagccagt

SEQ ID NO: 15 - DNA, 30 bp, synthetic construct
  misc_feature 1..30: note = morpholino antisense oligonucleotide
  source 1..30: mol_type = other DNA
ataccttctgcttgatgatcatctcgttga

SEQ ID NO: 16 - DNA, 22 bp, synthetic construct
  misc_feature 1..22: note = primer
  source 1..22: mol_type = other DNA
cagccagtgaagaggaagttag

SEQ ID NO: 17 - DNA, 20 bp, synthetic construct
  misc_feature 1..20: note = primer
  source 1..20: mol_type = other DNA
ccagccattgtgttgaatcc

SEQ ID NO: 18 - DNA, 20 bp, synthetic construct
  misc_feature 1..20: note = primer
  source 1..20: mol_type = other DNA
aggacccgtgcttgtaagtg

SEQ ID NO: 19 - DNA, 23 bp, synthetic construct
  misc_feature 1..23: note = primer
  source 1..23: mol_type = other DNA
gattgttctagcctcttgattgc

SEQ ID NO: 20 - DNA, 19 bp, synthetic construct
  misc_feature 1..19: note = primer
  source 1..19: mol_type = other DNA
gacaagggcgatttgacag

SEQ ID NO: 21 - DNA, 20 bp, synthetic construct
  misc_feature 1..20: note = primer
  source 1..20: mol_type = other DNA
caagcactcagccagtgaag

SEQ ID NO: 22 - DNA, 20 bp, synthetic construct
  misc_feature 1..20: note = primer
  source 1..20: mol_type = other DNA
tccagccattgtgttgaatc

SEQ ID NO: 23 - DNA, 20 bp, synthetic construct
  misc_feature 1..20: note = primer
  source 1..20: mol_type = other DNA
tccctgagctgaacgggaag

SEQ ID NO: 24 - DNA, 20 bp, synthetic construct
  misc_feature 1..20: note = primer
  source 1..20: mol_type = other DNA
tccagccattgtgttgaatc

SEQ ID NO: 25 - DNA, 23 bp, synthetic construct
  misc_feature 1..23: note = primer
  source 1..23: mol_type = other DNA
tcgatgctcttagctgagtgtcc

SEQ ID NO: 26 - DNA, 20 bp, synthetic construct
  misc_feature 1..20: note = primer
  source 1..20: mol_type = other DNA
tgatcgtcttcgaacctccg

SEQ ID NO: 27 - DNA, 30 bp, synthetic construct; identical to SEQ ID NO: 13
  misc_feature 1..30: note = morpholino antisense oligonucleotide
  source 1..30: mol_type = other DNA

SEQ ID NO: 28 - DNA, 30 bp, synthetic construct; identical to SEQ ID NO: 1
  misc_feature 1..30: note = morpholino antisense oligonucleotide
  source 1..30: mol_type = other DNA

SEQ ID NO: 29 - DNA, 30 bp, synthetic construct
  misc_feature 1..30: note = morpholino antisense oligonucleotide
  source 1..30: mol_type = other DNA
accacaggttgtgtcaccagagtaacagtc

SEQ ID NO: 30 - DNA, 30 bp, synthetic construct; identical to SEQ ID NO: 2
  misc_feature 1..30: note = morpholino antisense oligonucleotide
  source 1..30: mol_type = other DNA

SEQ ID NO: 31 - DNA, 30 bp, synthetic construct
  misc_feature 1..30: note = morpholino antisense oligonucleotide
  source 1..30: mol_type = other DNA
cacaggttgtgtcaccagagtaacagtctg

SEQ ID NO: 32 - DNA, 30 bp, synthetic construct
  misc_feature 1..30: note = morpholino antisense oligonucleotide
  source 1..30: mol_type = other DNA
tgtgtcaccagagtaacagtctgagtagga

SEQ ID NO: 33 - DNA, 30 bp, synthetic construct
  misc_feature 1..30: note = morpholino antisense oligonucleotide
  source 1..30: mol_type = other DNA
ttgtgtcaccagagtaacagtctgagtagg

SEQ ID NO: 34 - DNA, 30 bp, synthetic construct
  misc_feature 1..30: note = morpholino antisense oligonucleotide
  source 1..30: mol_type = other DNA
aaccacaggttgtgtcaccagagtaacagt

SEQ ID NO: 35 - DNA, 30 bp, synthetic construct
  misc_feature 1..30: note = morpholino antisense oligonucleotide
  source 1..30: mol_type = other DNA
caggttgtgtcaccagagtaacagtctgag

SEQ ID NO: 36 - DNA, 30 bp, synthetic construct
  misc_feature 1..30: note = morpholino antisense oligonucleotide
  source 1..30: mol_type = other DNA
acaggttgtgtcaccagagtaacagtctga

SEQ ID NO: 37 - DNA, 30 bp, synthetic construct
  misc_feature 1..30: note = morpholino antisense oligonucleotide
  source 1..30: mol_type = other DNA
ggttgtgtcaccagagtaacagtctgagta

SEQ ID NO: 38 - DNA, 30 bp, synthetic construct; identical to SEQ ID NO: 3
  misc_feature 1..30: note = morpholino antisense oligonucleotide
  source 1..30: mol_type = other DNA

SEQ ID NO: 39 - DNA, 30 bp, synthetic construct
  misc_feature 1..30: note = morpholino antisense oligonucleotide
  source 1..30: mol_type = other DNA
ccttagtaaccacaggttgtgtcaccagag

SEQ ID NO: 40 - DNA, 30 bp, synthetic construct
  misc_feature 1..30: note = morpholino antisense oligonucleotide
  source 1..30: mol_type = other DNA
tccttagtaaccacaggttgtgtcaccaga

SEQ ID NO: 41 - DNA, 30 bp, synthetic construct
  misc_feature 1..30: note = morpholino antisense oligonucleotide
  source 1..30: mol_type = other DNA
tggcagtttccttagtaaccacaggttgtg

SEQ ID NO: 42 - DNA, 30 bp, synthetic construct
  misc_feature 1..30: note = morpholino antisense oligonucleotide
  source 1..30: mol_type = other DNA
taaccacaggttgtgtcaccagagtaacag

SEQ ID NO: 43 - DNA, 30 bp, synthetic construct
  misc_feature 1..30: note = morpholino antisense oligonucleotide
  source 1..30: mol_type = other DNA
gtaaccacaggttgtgtcaccagagtaaca

SEQ ID NO: 44 - DNA, 30 bp, synthetic construct
  misc_feature 1..30: note = morpholino antisense oligonucleotide
  source 1..30: mol_type = other DNA
gcagtttccttagtaaccacaggttgtgtc

SEQ ID NO: 45 - DNA, 30 bp, synthetic construct
  misc_feature 1..30: note = morpholino antisense oligonucleotide
  source 1..30: mol_type = other DNA
gatggcagtttccttagtaaccacaggttg

SEQ ID NO: 46 - DNA, 30 bp, synthetic construct
  misc_feature 1..30: note = morpholino antisense oligonucleotide
  source 1..30: mol_type = other DNA
agtaaccacaggttgtgtcaccagagtaac

SEQ ID NO: 47 - DNA, 30 bp, synthetic construct
  misc_feature 1..30: note = morpholino antisense oligonucleotide
  source 1..30: mol_type = other DNA
agtttccttagtaaccacaggttgtgtcac

SEQ ID NO: 48 - DNA, 30 bp, synthetic construct
  misc_feature 1..30: note = morpholino antisense oligonucleotide
  source 1..30: mol_type = other DNA
gttgtgtcaccagagtaacagtctgagtag

SEQ ID NO: 49 - DNA, 30 bp, synthetic construct
  misc_feature 1..30: note = morpholino antisense oligonucleotide
  source 1..30: mol_type = other DNA
cttagtaaccacaggttgtgtcaccagagt

SEQ ID NO: 50 - DNA, 30 bp, synthetic construct
  misc_feature 1..30: note = morpholino antisense oligonucleotide
  source 1..30: mol_type = other DNA
cagtttccttagtaaccacaggttgtgtca

SEQ ID NO: 51 - DNA, 30 bp, synthetic construct; identical to SEQ ID NO: 4
  misc_feature 1..30: note = morpholino antisense oligonucleotide
  source 1..30: mol_type = other DNA

SEQ ID NO: 52 - DNA, 30 bp, synthetic construct
  misc_feature 1..30: note = morpholino antisense oligonucleotide
  source 1..30: mol_type = other DNA
ttccttagtaaccacaggttgtgtcaccag

SEQ ID NO: 53 - DNA, 30 bp, synthetic construct
  misc_feature 1..30: note = morpholino antisense oligonucleotide
  source 1..30: mol_type = other DNA
ttagtaaccacaggttgtgtcaccagagta

SEQ ID NO: 54 - DNA, 30 bp, synthetic construct
  misc_feature 1..30: note = morpholino antisense oligonucleotide
  source 1..30: mol_type = other DNA
tagtaaccacaggttgtgtcaccagagtaa

SEQ ID NO: 55 - DNA, 30 bp, synthetic construct
  misc_feature 1..30: note = morpholino antisense oligonucleotide
  source 1..30: mol_type = other DNA
gtttccttagtaaccacaggttgtgtcacc

SEQ ID NO: 56 - DNA, 30 bp, synthetic construct
  misc_feature 1..30: note = morpholino antisense oligonucleotide
  source 1..30: mol_type = other DNA
atggcagtttccttagtaaccacaggttgt

SEQ ID NO: 57 - DNA, 30 bp, synthetic construct
  misc_feature 1..30: note = morpholino antisense oligonucleotide
  source 1..30: mol_type = other DNA
tttccttagtaaccacaggttgtgtcacca

SEQ ID NO: 58 - DNA, 30 bp, synthetic construct
  misc_feature 1..30: note = morpholino antisense oligonucleotide
  source 1..30: mol_type = other DNA
gagatggcagtttccttagtaaccacaggt

SEQ ID NO: 59 - DNA, 30 bp, synthetic construct
  misc_feature 1..30: note = morpholino antisense oligonucleotide
  source 1..30: mol_type = other DNA
ggagatggcagtttccttagtaaccacagg

SEQ ID NO: 60 - DNA, 30 bp, synthetic construct
  misc_feature 1..30: note = morpholino antisense oligonucleotide
  source 1..30: mol_type = other DNA
ccaagcccggttgaaatctgccagagcagg

SEQ ID NO: 61 - DNA, 30 bp, synthetic construct
  misc_feature 1..30: note = morpholino antisense oligonucleotide
  source 1..30: mol_type = other DNA
cagagcaggtacctccaacatcaaggaaga

SEQ ID NO: 62 - DNA, 30 bp, synthetic construct
  misc_feature 1..30: note = morpholino antisense oligonucleotide
  source 1..30: mol_type = other DNA
ggcatttctagtttggagatggcagtttcc

SEQ ID NO: 63 - DNA, 30 bp, synthetic construct
  misc_feature 1..30: note = morpholino antisense oligonucleotide
  source 1..30: mol_type = other DNA
ctgtccaagcccggttgaaatctgccagag

SEQ ID NO: 64 - DNA, 30 bp, synthetic construct
  misc_feature 1..30: note = morpholino antisense oligonucleotide
  source 1..30: mol_type = other DNA
tctgtccaagcccggttgaaatctgccaga

SEQ ID NO: 65 - DNA, 30 bp, synthetic construct
  misc_feature 1..30: note = morpholino antisense oligonucleotide
  source 1..30: mol_type = other DNA
ccagagcaggtacctccaacatcaaggaag

SEQ ID NO: 66 - DNA, 30 bp, synthetic construct
  misc_feature 1..30: note = morpholino antisense oligonucleotide
  source 1..30: mol_type = other DNA
gtccaagcccggttgaaatctgccagagca

SEQ ID NO: 67 - DNA, 30 bp, synthetic construct
  misc_feature 1..30: note = morpholino antisense oligonucleotide
  source 1..30: mol_type = other DNA
tgtccaagcccggttgaaatctgccagagc

SEQ ID NO: 68 - DNA, 30 bp, synthetic construct
  misc_feature 1..30: note = morpholino antisense oligonucleotide
  source 1..30: mol_type = other DNA
ggaagatggcatttctagtttggagatggc

SEQ ID NO: 69 - DNA, 30 bp, synthetic construct
  misc_feature 1..30: note = morpholino antisense oligonucleotide
  source 1..30: mol_type = other DNA
tccaagcccggttgaaatctgccagagcag

SEQ ID NO: 70 - DNA, 30 bp, synthetic construct
  misc_feature 1..30: note = morpholino antisense oligonucleotide
  source 1..30: mol_type = other DNA
agttctgtccaagcccggttgaaatctgcc

SEQ ID NO: 71 - DNA, 30 bp, synthetic construct
  misc_feature 1..30: note = morpholino antisense oligonucleotide
  source 1..30: mol_type = other DNA
tggagatggcagtttccttagtaaccacag

SEQ ID NO: 72 - DNA, 30 bp, synthetic construct
  misc_feature 1..30: note = morpholino antisense oligonucleotide
  source 1..30: mol_type = other DNA
gttctgtccaagcccggttgaaatctgcca

SEQ ID NO: 73 - DNA, 30 bp, synthetic construct
  misc_feature 1..30: note = morpholino antisense oligonucleotide
  source 1..30: mol_type = other DNA
ttctgtccaagcccggttgaaatctgccag

SEQ ID NO: 74 - DNA, 30 bp, synthetic construct
  misc_feature 1..30: note = morpholino antisense oligonucleotide
  source 1..30: mol_type = other DNA
gccagtcggtaagttctgtccaagcccggt

SEQ ID NO: 75 - DNA, 30 bp, synthetic construct
  misc_feature 1..30: note = morpholino antisense oligonucleotide
  source 1..30: mol_type = other DNA
tgaaatctgccagagcaggtacctccaaca

SEQ ID NO: 76 - DNA, 30 bp, synthetic construct
  misc_feature 1..30: note = morpholino antisense oligonucleotide
  source 1..30: mol_type = other DNA
agtttggagatggcagtttccttagtaacc

SEQ ID NO: 77 - DNA, 30 bp, synthetic construct
  misc_feature 1..30: note = morpholino antisense oligonucleotide
  source 1..30: mol_type = other DNA
caagcccggttgaaatctgccagagcaggt

SEQ ID NO: 78 - DNA, 30 bp, synthetic construct
  misc_feature 1..30: note = morpholino antisense oligonucleotide
  source 1..30: mol_type = other DNA
tctagtttggagatggcagtttccttagta

SEQ ID NO: 79 - DNA, 30 bp, synthetic construct
  misc_feature 1..30: note = morpholino antisense oligonucleotide
  source 1..30: mol_type = other DNA
agagcaggtacctccaacatcaaggaagat

SEQ ID NO: 80 - DNA, 30 bp, synthetic construct
  misc_feature 1..30: note = morpholino antisense oligonucleotide
  source 1..30: mol_type = other DNA
ctgccagagcaggtacctccaacatcaagg

SEQ ID NO: 81 - DNA, 30 bp, synthetic construct
  misc_feature 1..30: note = morpholino antisense oligonucleotide
  source 1..30: mol_type = other DNA
agcccggttgaaatctgccagagcaggtac

SEQ ID NO: 82 - DNA, 30 bp, synthetic construct
  misc_feature 1..30: note = morpholino antisense oligonucleotide
  source 1..30: mol_type = other DNA
gaaatctgccagagcaggtacctccaacat

SEQ ID NO: 83 - DNA, 30 bp, synthetic construct
  misc_feature 1..30: note = morpholino antisense oligonucleotide
  source 1..30: mol_type = other DNA
tgccagagcaggtacctccaacatcaagga

SEQ ID NO: 84 - DNA, 30 bp, synthetic construct
  misc_feature 1..30: note = morpholino antisense oligonucleotide
  source 1..30: mol_type = other DNA
aagcccggttgaaatctgccagagcaggta

SEQ ID NO: 85 - DNA, 30 bp, synthetic construct
  misc_feature 1..30: note = morpholino antisense oligonucleotide
  source 1..30: mol_type = other DNA
gccagagcaggtacctccaacatcaaggaa

SEQ ID NO: 86 - DNA, 30 bp, synthetic construct
  misc_feature 1..30: note = morpholino antisense oligonucleotide
  source 1..30: mol_type = other DNA
taagttctgtccaagcccggttgaaatctg

SEQ ID NO: 87 - DNA, 30 bp, synthetic construct
  misc_feature 1..30: note = morpholino antisense oligonucleotide
  source 1..30: mol_type = other DNA
ggttgaaatctgccagagcaggtacctcca

SEQ ID NO: 88 - DNA, 30 bp, synthetic construct
  misc_feature 1..30: note = morpholino antisense oligonucleotide
  source 1..30: mol_type = other DNA
agatggcatttctagtttggagatggcagt

SEQ ID NO: 89 - DNA, 30 bp, synthetic construct
  misc_feature 1..30: note = morpholino antisense oligonucleotide
  source 1..30: mol_type = other DNA
gttgaaatctgccagagcaggtacctccaa

SEQ ID NO: 90 - DNA, 30 bp, synthetic construct
  misc_feature 1..30: note = morpholino antisense oligonucleotide
  source 1..30: mol_type = other DNA
gcccggttgaaatctgccagagcaggtacc

SEQ ID NO: 91 - DNA, 30 bp, synthetic construct
  misc_feature 1..30: note = morpholino antisense oligonucleotide
  source 1..30: mol_type = other DNA
ttgaaatctgccagagcaggtacctccaac

SEQ ID NO: 92 - DNA, 30 bp, synthetic construct
  misc_feature 1..30: note = morpholino antisense oligonucleotide
  source 1..30: mol_type = other DNA
ttggagatggcagtttccttagtaaccaca

SEQ ID NO: 93 - DNA, 30 bp, synthetic construct; identical to SEQ ID NO: 6
  misc_feature 1..30: note = morpholino antisense oligonucleotide
  source 1..30: mol_type = other DNA

SEQ ID NO: 94 - DNA, 30 bp, synthetic construct
  misc_feature 1..30: note = morpholino antisense oligonucleotide
  source 1..30: mol_type = other DNA
tttggagatggcagtttccttagtaaccac

SEQ ID NO: 95 - DNA, 30 bp, synthetic construct
  misc_feature 1..30: note = morpholino antisense oligonucleotide
  source 1..30: mol_type = other DNA
cggttgaaatctgccagagcaggtacctcc

SEQ ID NO: 96 - DNA, 30 bp, synthetic construct
  misc_feature 1..30: note = morpholino antisense oligonucleotide
  source 1..30: mol_type = other DNA
gcatttctagtttggagatggcagtttcct

SEQ ID NO: 97 - DNA, 30 bp, synthetic construct
  misc_feature 1..30: note = morpholino antisense oligonucleotide
  source 1..30: mol_type = other DNA
tcaaggaagatggcatttctagtttggaga

SEQ ID NO: 98 - DNA, 30 bp, synthetic construct
  misc_feature 1..30: note = morpholino antisense oligonucleotide
  source 1..30: mol_type = other DNA
agccagtcggtaagttctgtccaagcccgg

SEQ ID NO: 99 - DNA, 30 bp, synthetic construct
  misc_feature 1..30: note = morpholino antisense oligonucleotide
  source 1..30: mol_type = other DNA
tctgccagagcaggtacctccaacatcaag

SEQ ID NO: 100 - DNA, 30 bp, synthetic construct
  misc_feature 1..30: note = morpholino antisense oligonucleotide
  source 1..30: mol_type = other DNA
cccggttgaaatctgccagagcaggtacct

SEQ ID NO: 101 - DNA, 30 bp, synthetic construct
  misc_feature 1..30: note = morpholino antisense oligonucleotide
  source 1..30: mol_type = other DNA
aagttctgtccaagcccggttgaaatctgc

SEQ ID NO: 102 - DNA, 30 bp, synthetic construct
  misc_feature 1..30: note = morpholino antisense oligonucleotide
  source 1..30: mol_type = other DNA
ccagtcggtaagttctgtccaagcccggtt

SEQ ID NO: 103 - DNA, 30 bp, synthetic construct
  misc_feature 1..30: note = morpholino antisense oligonucleotide
  source 1..30: mol_type = other DNA
gtaagttctgtccaagcccggttgaaatct

SEQ ID NO: 104 - DNA, 30 bp, synthetic construct
  misc_feature 1..30: note = morpholino antisense oligonucleotide
  source 1..30: mol_type = other DNA
ggtaagttctgtccaagcccggttgaaatc

SEQ ID NO: 105 - DNA, 30 bp, synthetic construct
  misc_feature 1..30: note = morpholino antisense oligonucleotide
  source 1..30: mol_type = other DNA
ccggttgaaatctgccagagcaggtacctc

SEQ ID NO: 106 - DNA, 30 bp, synthetic construct
  misc_feature 1..30: note = morpholino antisense oligonucleotide
  source 1..30: mol_type = other DNA
gtttggagatggcagtttccttagtaacca

SEQ ID NO: 107 - DNA, 30 bp, synthetic construct
  misc_feature 1..30: note = morpholino antisense oligonucleotide
  source 1..30: mol_type = other DNA
caaggaagatggcatttctagtttggagat

SEQ ID NO: 108 - DNA, 30 bp, synthetic construct
  misc_feature 1..30: note = morpholino antisense oligonucleotide
  source 1..30: mol_type = other DNA
gagcaggtacctccaacatcaaggaagatg

SEQ ID NO: 109 - DNA, 30 bp, synthetic construct
  misc_feature 1..30: note = morpholino antisense oligonucleotide
  source 1..30: mol_type = other DNA
ctagtttggagatggcagtttccttagtaa

SEQ ID NO: 110 - DNA, 30 bp, synthetic construct
  misc_feature 1..30: note = morpholino antisense oligonucleotide
  source 1..30: mol_type = other DNA
tccaacatcaaggaagatggcatttctagt

SEQ ID NO: 111 - DNA, 30 bp, synthetic construct
  misc_feature 1..30: note = morpholino antisense oligonucleotide
  source 1..30: mol_type = other DNA
cagtcggtaagttctgtccaagcccggttg

SEQ ID NO: 112 - DNA, 30 bp, synthetic construct
  misc_feature 1..30: note = morpholino antisense oligonucleotide
  source 1..30: mol_type = other DNA
gaagatggcatttctagtttggagatggca

SEQ ID NO: 113 - DNA, 30 bp, synthetic construct
  misc_feature 1..30: note = morpholino antisense oligonucleotide
  source 1..30: mol_type = other DNA
ccaacatcaaggaagatggcatttctagtt

SEQ ID NO: 114 - DNA, 30 bp, synthetic construct
  misc_feature 1..30: note = morpholino antisense oligonucleotide
  source 1..30: mol_type = other DNA
cggtaagttctgtccaagcccggttgaaat

SEQ ID NO: 115 - DNA, 30 bp, synthetic construct
  misc_feature 1..30: note = morpholino antisense oligonucleotide
  source 1..30: mol_type = other DNA
ttctagtttggagatggcagtttccttagt

SEQ ID NO: 116 - DNA, 30 bp, synthetic construct
  misc_feature 1..30: note = morpholino antisense oligonucleotide
  source 1..30: mol_type = other DNA
aatctgccagagcaggtacctccaacatca

SEQ ID NO: 117 - DNA, 30 bp, synthetic construct
  misc_feature 1..30: note = morpholino antisense oligonucleotide
  source 1..30: mol_type = other DNA
gtcggtaagttctgtccaagcccggttgaa

SEQ ID NO: 118 - DNA, 30 bp, synthetic construct
  misc_feature 1..30: note = morpholino antisense oligonucleotide
  source 1..30: mol_type = other DNA
agtcggtaagttctgtccaagcccggttga

SEQ ID NO: 119 - DNA, 30 bp, synthetic construct
  misc_feature 1..30: note = morpholino antisense oligonucleotide
  source 1..30: mol_type = other DNA
gcaggtacctccaacatcaaggaagatggc

SEQ ID NO: 120 - DNA, 30 bp, synthetic construct
  misc_feature 1..30: note = morpholino antisense oligonucleotide
  source 1..30: mol_type = other DNA
tagtttggagatggcagtttccttagtaac

SEQ ID NO: 121 - DNA, 30 bp, synthetic construct
  misc_feature 1..30: note = morpholino antisense oligonucleotide
  source 1..30: mol_type = other DNA
aagccagtcggtaagttctgtccaagcccg

SEQ ID NO: 122 - DNA, 30 bp, synthetic construct
  misc_feature 1..30: note = morpholino antisense oligonucleotide
  source 1..30: mol_type = other DNA
acctccaacatcaaggaagatggcatttct

SEQ ID NO: 123 - DNA, 30 bp, synthetic construct
  misc_feature 1..30: note = morpholino antisense oligonucleotide
  source 1..30: mol_type = other DNA
atctgccagagcaggtacctccaacatcaa

SEQ ID NO: 124 - DNA, 30 bp, synthetic construct
  misc_feature 1..30: note = morpholino antisense oligonucleotide
  source 1..30: mol_type = other DNA
atcaaggaagatggcatttctagtttggag

SEQ ID NO: 125 - DNA, 30 bp, synthetic construct
  misc_feature 1..30: note = morpholino antisense oligonucleotide
  source 1..30: mol_type = other DNA
tcggtaagttctgtccaagcccggttgaaa

SEQ ID NO: 126 - DNA, 30 bp, synthetic construct
  misc_feature 1..30: note = morpholino antisense oligonucleotide
  source 1..30: mol_type = other DNA
aggaagatggcatttctagtttggagatgg

SEQ ID NO: 127 - DNA, 30 bp, synthetic construct
  misc_feature 1..30: note = morpholino antisense oligonucleotide
  source 1..30: mol_type = other DNA
aaatctgccagagcaggtacctccaacatc

SEQ ID NO: 128 - DNA, 30 bp, synthetic construct
  misc_feature 1..30: note = morpholino antisense oligonucleotide
  source 1..30: mol_type = other DNA
cctccaacatcaaggaagatggcatttcta

SEQ ID NO: 129 - DNA, 30 bp, synthetic construct
  misc_feature 1..30: note = morpholino antisense oligonucleotide
  source 1..30: mol_type = other DNA
gatggcatttctagtttggagatggcagtt

SEQ ID NO: 130 - DNA, 30 bp, synthetic construct
  misc_feature 1..30: note = morpholino antisense oligonucleotide
  source 1..30: mol_type = other DNA
tttctagtttggagatggcagtttccttag

SEQ ID NO: 131 - DNA, 30 bp, synthetic construct
  misc_feature 1..30: note = morpholino antisense oligonucleotide
  source 1..30: mol_type = other DNA
catttctagtttggagatggcagtttcctt

SEQ ID NO: 132 - DNA, 30 bp, synthetic construct
  misc_feature 1..30: note = morpholino antisense oligonucleotide
  source 1..30: mol_type = other DNA
aagatggcatttctagtttggagatggcag

SEQ ID NO: 133 - DNA, 30 bp, synthetic construct
  misc_feature 1..30: note = morpholino antisense oligonucleotide
  source 1..30: mol_type = other DNA
aaggaagatggcatttctagtttggagatg

SEQ ID NO: 134 - DNA, 30 bp, synthetic construct
  misc_feature 1..30: note = morpholino antisense oligonucleotide
  source 1..30: mol_type = other DNA
tacctccaacatcaaggaagatggcatttc

SEQ ID NO: 135 - DNA, 30 bp, synthetic construct
  misc_feature 1..30: note = morpholino antisense oligonucleotide
  source 1..30: mol_type = other DNA
gaaagccagtcggtaagttctgtccaagcc

SEQ ID NO: 136 - DNA, 30 bp, synthetic construct
  misc_feature 1..30: note = morpholino antisense oligonucleotide
  source 1..30: mol_type = other DNA
aaagccagtcggtaagttctgtccaagccc

SEQ ID NO: 137 - DNA, 30 bp, synthetic construct
  misc_feature 1..30: note = morpholino antisense oligonucleotide
  source 1..30: mol_type = other DNA
agcaggtacctccaacatcaaggaagatgg

SEQ ID NO: 138 - DNA, 30 bp, synthetic construct
  misc_feature 1..30: note = morpholino antisense oligonucleotide
  source 1..30: mol_type = other DNA
agaaagccagtcggtaagttctgtccaagc

SEQ ID NO: 139 - DNA, 30 bp, synthetic construct
  misc_feature 1..30: note = morpholino antisense oligonucleotide
  source 1..30: mol_type = other DNA
catcaaggaagatggcatttctagtttgga

SEQ ID NO: 140 - DNA, 30 bp, synthetic construct
  misc_feature 1..30: note = morpholino antisense oligonucleotide
  source 1..30: mol_type = other DNA
gagaaagccagtcggtaagttctgtccaag

SEQ ID NO: 141 - DNA, 30 bp, synthetic construct
  misc_feature 1..30: note = morpholino antisense oligonucleotide
  source 1..30: mol_type = other DNA
gtacctccaacatcaaggaagatggcattt

SEQ ID NO: 142 - DNA, 30 bp, synthetic construct
  misc_feature 1..30: note = morpholino antisense oligonucleotide
  source 1..30: mol_type = other DNA
acatcaaggaagatggcatttctagtttgg

SEQ ID NO: 143 - DNA, 30 bp, synthetic construct
  misc_feature 1..30: note = morpholino antisense oligonucleotide
  source 1..30: mol_type = other DNA
caggtacctccaacatcaaggaagatggca

SEQ ID NO: 144 - DNA, 30 bp, synthetic construct
  misc_feature 1..30: note = morpholino antisense oligonucleotide
  source 1..30: mol_type = other DNA
agagaaagccagtcggtaagttctgtccaa

SEQ ID NO: 145 - DNA, 30 bp, synthetic construct
  misc_feature 1..30: note = morpholino antisense oligonucleotide
  source 1..30: mol_type = other DNA
ggtacctccaacatcaaggaagatggcatt

SEQ ID NO: 146 - DNA, 30 bp, synthetic construct
  misc_feature 1..30: note = morpholino antisense oligonucleotide
  source 1..30: mol_type = other DNA
aggtacctccaacatcaaggaagatggcat

SEQ ID NO: 147 - DNA, 30 bp, synthetic construct
  misc_feature 1..30: note = morpholino antisense oligonucleotide
  source 1..30: mol_type = other DNA
caacatcaaggaagatggcatttctagttt

SEQ ID NO: 148 - DNA, 30 bp, synthetic construct
  misc_feature 1..30: note = morpholino antisense oligonucleotide
  source 1..30: mol_type = other DNA
aacatcaaggaagatggcatttctagtttg

SEQ ID NO: 149 - DNA, 30 bp, synthetic construct
  misc_feature 1..30: note = morpholino antisense oligonucleotide
  source 1..30: mol_type = other DNA
gcagagaaagccagtcggtaagttctgtcc

SEQ ID NO: 150 - DNA, 30 bp, synthetic construct
  misc_feature 1..30: note = morpholino antisense oligonucleotide
  source 1..30: mol_type = other DNA
cagagaaagccagtcggtaagttctgtcca

SEQ ID NO: 151 - DNA, 30 bp, synthetic construct
  misc_feature 1..30: note = morpholino antisense oligonucleotide
  source 1..30: mol_type = other DNA
atttctagtttggagatggcagtttcctta

SEQ ID NO: 152 - DNA, 30 bp, synthetic construct
  misc_feature 1..30: note = morpholino antisense oligonucleotide
  source 1..30: mol_type = other DNA
tggcatttctagtttggagatggcagtttc

SEQ ID NO: 153 - DNA, 30 bp, synthetic construct
  misc_feature 1..30: note = morpholino antisense oligonucleotide
  source 1..30: mol_type = other DNA
caagcagagaaagccagtcggtaagttctg

SEQ ID NO: 154 - DNA, 30 bp, synthetic construct; identical to SEQ ID NO: 5
  misc_feature 1..30: note = morpholino antisense oligonucleotide
  source 1..30: mol_type = other DNA

SEQ ID NO: 155 - DNA, 30 bp, synthetic construct
  misc_feature 1..30: note = morpholino antisense oligonucleotide
  source 1..30: mol_type = other DNA
tcaagcagagaaagccagtcggtaagttct

SEQ ID NO: 156 - DNA, 30 bp, synthetic construct
  misc_feature 1..30: note = morpholino antisense oligonucleotide
  source 1..30: mol_type = other DNA
aagcagagaaagccagtcggtaagttctgt

SEQ ID NO: 157 - DNA, 30 bp, synthetic construct
  misc_feature 1..30: note = morpholino antisense oligonucleotide
  source 1..30: mol_type = other DNA
atcaagcagagaaagccagtcggtaagttc

SEQ ID NO: 158 - DNA, 30 bp, synthetic construct
  misc_feature 1..30: note = morpholino antisense oligonucleotide
  source 1..30: mol_type = other DNA
agcagagaaagccagtcggtaagttctgtc

SEQ ID NO: 159 - DNA, 30 bp, synthetic construct
  misc_feature 1..30: note = morpholino antisense oligonucleotide
  source 1..30: mol_type = other DNA
acttgatcaagcagagaaagccagtcggta

SEQ ID NO: 160 - DNA, 30 bp, synthetic construct
  misc_feature 1..30: note = morpholino antisense oligonucleotide
  source 1..30: mol_type = other DNA
aacttgatcaagcagagaaagccagtcggt

SEQ ID NO: 161 - DNA, 30 bp, synthetic construct
  misc_feature 1..30: note = morpholino antisense oligonucleotide
  source 1..30: mol_type = other DNA
taacttgatcaagcagagaaagccagtcgg

SEQ ID NO: 162 - DNA, 30 bp, synthetic construct
  misc_feature 1..30: note = morpholino antisense oligonucleotide
  source 1..30: mol_type = other DNA
gatcaagcagagaaagccagtcggtaagtt

SEQ ID NO: 163 - DNA, 30 bp, synthetic construct
  misc_feature 1..30: note = morpholino antisense oligonucleotide
  source 1..30: mol_type = other DNA
tataacttgatcaagcagagaaagccagtc

SEQ ID NO: 164 - DNA, 30 bp, synthetic construct
  misc_feature 1..30: note = morpholino antisense oligonucleotide
  source 1..30: mol_type = other DNA
tgatcaagcagagaaagccagtcggtaagt

SEQ ID NO: 165 - DNA, 30 bp, synthetic construct
  misc_feature 1..30: note = morpholino antisense oligonucleotide
  source 1..30: mol_type = other DNA
ttgatcaagcagagaaagccagtcggtaag

SEQ ID NO: 166 - DNA, 30 bp, synthetic construct
  misc_feature 1..30: note = morpholino antisense oligonucleotide
  source 1..30: mol_type = other DNA
ataacttgatcaagcagagaaagccagtcg

SEQ ID NO: 167 - DNA, 30 bp, synthetic construct
  misc_feature 1..30: note = morpholino antisense oligonucleotide
  source 1..30: mol_type = other DNA
cttgatcaagcagagaaagccagtcggtaa

SEQ ID NO: 168 - DNA, 30 bp, synthetic construct; identical to SEQ ID NO: 14
  misc_feature 1..30: note = morpholino antisense oligonucleotide
  source 1..30: mol_type = other DNA

SEQ ID NO: 169 - DNA, 30 bp, synthetic construct
  misc_feature 1..30: note = morpholino antisense oligonucleotide
  source 1..30: mol_type = other DNA
tttataacttgatcaagcagagaaagccag

SEQ ID NO: 170 - DNA, 30 bp, synthetic construct
  misc_feature 1..30: note = morpholino antisense oligonucleotide
  source 1..30: mol_type = other DNA
tgattttataacttgatcaagcagagaaag

SEQ ID NO: 171 - DNA, 30 bp, synthetic construct
  misc_feature 1..30: note = morpholino antisense oligonucleotide
  source 1..30: mol_type = other DNA
gattttataacttgatcaagcagagaaagc

SEQ ID NO: 172 - DNA, 30 bp, synthetic construct
  misc_feature 1..30: note = morpholino antisense oligonucleotide
  source 1..30: mol_type = other DNA
tcaccctctgtgattttataacttgatcaa

SEQ ID NO: 173 - DNA, 30 bp, synthetic construct
  misc_feature 1..30: note = morpholino antisense oligonucleotide
  source 1..30: mol_type = other DNA
gtcacccaccatcaccctctgtgattttat

SEQ ID NO: 174 - DNA, 30 bp, synthetic construct
  misc_feature 1..30: note = morpholino antisense oligonucleotide
  source 1..30: mol_type = other DNA
ttttataacttgatcaagcagagaaagcca

SEQ ID NO: 175 - DNA, 30 bp, synthetic construct
  misc_feature 1..30: note = morpholino antisense oligonucleotide
  source 1..30: mol_type = other DNA
cctcaaggtcacccaccatcaccctctgtg

SEQ ID NO: 176 - DNA, 30 bp, synthetic construct
  misc_feature 1..30: note = morpholino antisense oligonucleotide
  source 1..30: mol_type = other DNA
attttataacttgatcaagcagagaaagcc

SEQ ID NO: 177 - DNA, 30 bp, synthetic construct
  misc_feature 1..30: note = morpholino antisense oligonucleotide
  source 1..30: mol_type = other DNA
caccctctgtgattttataacttgatcaag

SEQ ID NO: 178 - DNA, 30 bp, synthetic construct
  misc_feature 1..30: note = morpholino antisense oligonucleotide
  source 1..30: mol_type = other DNA
ggtcacccaccatcaccctctgtgatttta

SEQ ID NO: 179 - DNA, 30 bp, synthetic construct
  misc_feature 1..30: note = morpholino antisense oligonucleotide
  source 1..30: mol_type = other DNA
tcctcaaggtcacccaccatcaccctctgt

SEQ ID NO: 180 - DNA, 30 bp, synthetic construct
  misc_feature 1..30: note = morpholino antisense oligonucleotide
  source 1..30: mol_type = other DNA
atcaccctctgtgattttataacttgatca

SEQ ID NO: 181 - DNA, 30 bp, synthetic construct
  misc_feature 1..30: note = morpholino antisense oligonucleotide
  source 1..30: mol_type = other DNA
ctcaaggtcacccaccatcaccctctgtga

SEQ ID NO: 182 - DNA, 30 bp, synthetic construct
  misc_feature 1..30: note = morpholino antisense oligonucleotide
  source 1..30: mol_type = other DNA
atcctcaaggtcacccaccatcaccctctg

SEQ ID NO: 183 - DNA, 30 bp, synthetic construct
  misc_feature 1..30: note = morpholino antisense oligonucleotide
  source 1..30: mol_type = other DNA
tcaaggtcacccaccatcaccctctgtgat

SEQ ID NO: 184 - DNA, 30 bp, synthetic construct
  misc_feature 1..30: note = morpholino antisense oligonucleotide
  source 1..30: mol_type = other DNA
tcacccaccatcaccctctgtgattttata

SEQ ID NO: 185 - DNA, 30 bp, synthetic construct
  misc_feature 1..30: note = morpholino antisense oligonucleotide
  source 1..30: mol_type = other DNA
cccaccatcaccctctgtgattttataact

SEQ ID NO: 186 - DNA, 30 bp, synthetic construct
  misc_feature 1..30: note = morpholino antisense oligonucleotide
  source 1..30: mol_type = other DNA
caccatcaccctctgtgattttataacttg

SEQ ID NO: 187 - DNA, 30 bp, synthetic construct
  misc_feature 1..30: note = morpholino antisense oligonucleotide
  source 1..30: mol_type = other DNA
gtgattttataacttgatcaagcagagaaa

SEQ ID NO: 188 - DNA, 30 bp, synthetic construct
  misc_feature 1..30: note = morpholino antisense oligonucleotide
  source 1..30: mol_type = other DNA
acccaccatcaccctctgtgattttataac

SEQ ID NO: 189 - DNA, 30 bp, synthetic construct
  misc_feature 1..30: note = morpholino antisense oligonucleotide
  source 1..30: mol_type = other DNA
caaggtcacccaccatcaccctctgtgatt

SEQ ID NO: 190 - DNA, 30 bp, synthetic construct
  misc_feature 1..30: note = morpholino antisense oligonucleotide
  source 1..30: mol_type = other DNA
aggtcacccaccatcaccctctgtgatttt

SEQ ID NO: 191 - DNA, 30 bp, synthetic construct
  misc_feature 1..30: note = morpholino antisense oligonucleotide
  source 1..30: mol_type = other DNA
ccctctgtgattttataacttgatcaagca

SEQ ID NO: 192 - DNA, 30 bp, synthetic construct
  misc_feature 1..30: note = morpholino antisense oligonucleotide
  source 1..30: mol_type = other DNA
cacccaccatcaccctctgtgattttataa

SEQ ID NO: 193 - DNA, 30 bp, synthetic construct
  misc_feature 1..30: note = morpholino antisense oligonucleotide
  source 1..30: mol_type = other DNA
atatcctcaaggtcacccaccatcaccctc

SEQ ID NO: 194 - DNA, 30 bp, synthetic construct
  misc_feature 1..30: note = morpholino antisense oligonucleotide
  source 1..30: mol_type = other DNA
accatcaccctctgtgattttataacttga

SEQ ID NO: 195 - DNA, 30 bp, synthetic construct
  misc_feature 1..30: note = morpholino antisense oligonucleotide
  source 1..30: mol_type = other DNA
tatcctcaaggtcacccaccatcaccctct

SEQ ID NO: 196 - DNA, 30 bp, synthetic construct
  misc_feature 1..30: note = morpholino antisense oligonucleotide
  source 1..30: mol_type = other DNA
accctctgtgattttataacttgatcaagc

SEQ ID NO: 197 - DNA, 30 bp, synthetic construct
  misc_feature 1..30: note = morpholino antisense oligonucleotide
  source 1..30: mol_type = other DNA
aaggtcacccaccatcaccctctgtgattt

SEQ ID NO: 198 - DNA, 30 bp, synthetic construct
  misc_feature 1..30: note = morpholino antisense oligonucleotide
  source 1..30: mol_type = other DNA
ccaccatcaccctctgtgattttataactt

SEQ ID NO: 199 - DNA, 30 bp, synthetic construct
  misc_feature 1..30: note = morpholino antisense oligonucleotide
  source 1..30: mol_type = other DNA
ccatcaccctctgtgattttataacttgat

SEQ ID NO: 200 - DNA, 30 bp, synthetic construct
  misc_feature 1..30: note = morpholino antisense oligonucleotide
  source 1..30: mol_type = other DNA
catcaccctctgtgattttataacttgatc

SEQ ID NO: 201 - DNA, 30 bp, synthetic construct
  misc_feature 1..30: note = morpholino antisense oligonucleotide
  source 1..30: mol_type = other DNA
gatatcctcaaggtcacccaccatcaccct

SEQ ID NO: 202 - DNA, 30 bp, synthetic construct
  misc_feature 1..30: note = morpholino antisense oligonucleotide
  source 1..30: mol_type = other DNA
cctctgtgattttataacttgatcaagcag

SEQ ID NO: 203 - DNA, 30 bp, synthetic construct
  misc_feature 1..30: note = morpholino antisense oligonucleotide
  source 1..30: mol_type = other DNA
ctgtgattttataacttgatcaagcagaga

SEQ ID NO: 204 - DNA, 30 bp, synthetic construct
  misc_feature 1..30: note = morpholino antisense oligonucleotide
  source 1..30: mol_type = other DNA
tgatatcctcaaggtcacccaccatcaccc

SEQ ID NO: 205 - DNA, 30 bp, synthetic construct
  misc_feature 1..30: note = morpholino antisense oligonucleotide
  source 1..30: mol_type = other DNA
ttgatatcctcaaggtcacccaccatcacc

SEQ ID NO: 206 - DNA, 30 bp, synthetic construct
  misc_feature 1..30: note = morpholino antisense oligonucleotide
  source 1..30: mol_type = other DNA
tgtgattttataacttgatcaagcagagaa

SEQ ID NO: 207 - DNA, 30 bp, synthetic construct
  misc_feature 1..30: note = morpholino antisense oligonucleotide
  source 1..30: mol_type = other DNA
gttgatatcctcaaggtcacccaccatcac

SEQ ID NO: 208 - DNA, 30 bp, synthetic construct
  misc_feature 1..30: note = morpholino antisense oligonucleotide
  source 1..30: mol_type = other DNA
cgttgatatcctcaaggtcacccaccatca

SEQ ID NO: 209 - DNA, 30 bp, synthetic construct
  misc_feature 1..30: note = morpholino antisense oligonucleotide
  source 1..30: mol_type = other DNA
tcgttgatatcctcaaggtcacccaccatc

SEQ ID NO: 210 - DNA, 30 bp, synthetic construct
  misc_feature 1..30: note = morpholino antisense oligonucleotide
  source 1..30: mol_type = other DNA
ctcgttgatatcctcaaggtcacccaccat

SEQ ID NO: 211 - DNA, 30 bp, synthetic construct
  misc_feature 1..30: note = morpholino antisense oligonucleotide
  source 1..30: mol_type = other DNA
tctcgttgatatcctcaaggtcacccacca

SEQ ID NO: 212 - DNA, 30 bp, synthetic construct
  misc_feature 1..30: note = morpholino antisense oligonucleotide
  source 1..30: mol_type = other DNA
catctcgttgatatcctcaaggtcacccac

SEQ ID NO: 213 - DNA, 30 bp, synthetic construct
  misc_feature 1..30: note = morpholino antisense oligonucleotide
  source 1..30: mol_type = other DNA
tcatctcgttgatatcctcaaggtcaccca

SEQ ID NO: 214 - DNA, 30 bp, synthetic construct
  misc_feature 1..30: note = morpholino antisense oligonucleotide
  source 1..30: mol_type = other DNA
atctcgttgatatcctcaaggtcacccacc

SEQ ID NO: 215 - DNA, 30 bp, synthetic construct
  misc_feature 1..30: note = morpholino antisense oligonucleotide
  source 1..30: mol_type = other DNA
atcatctcgttgatatcctcaaggtcaccc

SEQ ID NO: 216 - DNA, 30 bp, synthetic construct
  misc_feature 1..30: note = morpholino antisense oligonucleotide
  source 1..30: mol_type = other DNA
gatcatctcgttgatatcctcaaggtcacc

SEQ ID NO: 217 - DNA, 30 bp, synthetic construct
  misc_feature 1..30: note = morpholino antisense oligonucleotide
  source 1..30: mol_type = other DNA
ctctgtgattttataacttgatcaagcaga

SEQ ID NO: 218 - DNA, 30 bp, synthetic construct
  misc_feature 1..30: note = morpholino antisense oligonucleotide
  source 1..30: mol_type = other DNA
tgatcatctcgttgatatcctcaaggtcac

SEQ ID NO: 219 - DNA, 30 bp, synthetic construct
  misc_feature 1..30: note = morpholino antisense oligonucleotide
  source 1..30: mol_type = other DNA
tctgtgattttataacttgatcaagcagag

SEQ ID NO: 220 - DNA, 30 bp, synthetic construct
  misc_feature 1..30: note = morpholino antisense oligonucleotide
  source 1..30: mol_type = other DNA
atgatcatctcgttgatatcctcaaggtca

SEQ ID NO: 221 - DNA, 30 bp, synthetic construct
  misc_feature 1..30: note = morpholino antisense oligonucleotide
  source 1..30: mol_type = other DNA
gatgatcatctcgttgatatcctcaaggtc

SEQ ID NO: 222 - DNA, 30 bp, synthetic construct
  misc_feature 1..30: note = morpholino antisense oligonucleotide
  source 1..30: mol_type = other DNA
tgatgatcatctcgttgatatcctcaaggt

SEQ ID NO: 223 - DNA, 30 bp, synthetic construct
  misc_feature 1..30: note = morpholino antisense oligonucleotide
  source 1..30: mol_type = other DNA
ttgatgatcatctcgttgatatcctcaagg

SEQ ID NO: 224 - DNA, 30 bp, synthetic construct
  misc_feature 1..30: note = morpholino antisense oligonucleotide
  source 1..30: mol_type = other DNA
gcttgatgatcatctcgttgatatcctcaa

SEQ ID NO: 225 - DNA, 30 bp, synthetic construct
  misc_feature 1..30: note = morpholino antisense oligonucleotide
  source 1..30: mol_type = other DNA
cttgatgatcatctcgttgatatcctcaag

SEQ ID NO: 226 - DNA, 30 bp, synthetic construct
  misc_feature 1..30: note = morpholino antisense oligonucleotide
  source 1..30: mol_type = other DNA
tgcttgatgatcatctcgttgatatcctca

SEQ ID NO: 227 - DNA, 30 bp, synthetic construct
  misc_feature 1..30: note = morpholino antisense oligonucleotide
  source 1..30: mol_type = other DNA
ctgcttgatgatcatctcgttgatatcctc

SEQ ID NO: 228 - DNA, 30 bp, synthetic construct
  misc_feature 1..30: note = morpholino antisense oligonucleotide
  source 1..30: mol_type = other DNA
tctgcttgatgatcatctcgttgatatcct

SEQ ID NO: 229 - DNA, 30 bp, synthetic construct
  misc_feature 1..30: note = morpholino antisense oligonucleotide
  source 1..30: mol_type = other DNA
ttctgcttgatgatcatctcgttgatatcc

SEQ ID NO: 230 - DNA, 30 bp, synthetic construct
  misc_feature 1..30: note = morpholino antisense oligonucleotide
  source 1..30: mol_type = other DNA
cttctgcttgatgatcatctcgttgatatc

SEQ ID NO: 231 - DNA, 25 bp, synthetic construct
  misc_feature 1..25: note = morpholino antisense oligonucleotide
  source 1..25: mol_type = other DNA
gtaaccacaggttgtgtcaccagag

SEQ ID NO: 232 - DNA, 25 bp, synthetic construct
  misc_feature 1..25: note = morpholino antisense oligonucleotide
  source 1..25: mol_type = other DNA
aaccacaggttgtgtcaccagagta

SEQ ID NO: 233 - DNA, 25 bp, synthetic construct
  misc_feature 1..25: note = morpholino antisense oligonucleotide
  source 1..25: mol_type = other DNA
acaggttgtgtcaccagagtaacag

SEQ ID NO: 234 - DNA, 25 bp, synthetic construct
  misc_feature 1..25: note = morpholino antisense oligonucleotide
  source 1..25: mol_type = other DNA
ccacaggttgtgtcaccagagtaac

SEQ ID NO: 235 - DNA, 25 bp, synthetic construct
  misc_feature 1..25: note = morpholino antisense oligonucleotide
  source 1..25: mol_type = other DNA
accacaggttgtgtcaccagagtaa

SEQ ID NO: 236 - DNA, 25 bp, synthetic construct
  misc_feature 1..25: note = morpholino antisense oligonucleotide
  source 1..25: mol_type = other DNA
ggcagtttccttagtaaccacaggt

SEQ ID NO: 237 - DNA, 25 bp, synthetic construct
  misc_feature 1..25: note = morpholino antisense oligonucleotide
  source 1..25: mol_type = other DNA
cacaggttgtgtcaccagagtaaca

SEQ ID NO: 238 - DNA, 25 bp, synthetic construct
  misc_feature 1..25: note = morpholino antisense oligonucleotide
  source 1..25: mol_type = other DNA
aggttgtgtcaccagagtaacagtc

SEQ ID NO: 239 - DNA, 25 bp, synthetic construct
  misc_feature 1..25: note = morpholino antisense oligonucleotide
  source 1..25: mol_type = other DNA
caggttgtgtcaccagagtaacagt

SEQ ID NO: 240 - DNA, 25 bp, synthetic construct
  misc_feature 1..25: note = morpholino antisense oligonucleotide
  source 1..25: mol_type = other DNA
accagagtaacagtctgagtaggag

SEQ ID NO: 241 - DNA, 25 bp, synthetic construct
  misc_feature 1..25: note = morpholino antisense oligonucleotide
  source 1..25: mol_type = other DNA
ggttgtgtcaccagagtaacagtct

SEQ ID NO: 242 - DNA, 25 bp, synthetic construct
  misc_feature 1..25: note = morpholino antisense oligonucleotide
  source 1..25: mol_type = other DNA
taaccacaggttgtgtcaccagagt

SEQ ID NO: 243 - DNA, 25 bp, synthetic construct
  misc_feature 1..25: note = morpholino antisense oligonucleotide
  source 1..25: mol_type = other DNA
tggcagtttccttagtaaccacagg

SEQ ID NO: 244 - DNA, 25 bp, synthetic construct
  misc_feature 1..25: note = morpholino antisense oligonucleotide
  source 1..25: mol_type = other DNA
gcagtttccttagtaaccacaggtt

SEQ ID NO: 245 - DNA, 25 bp, synthetic construct
  misc_feature 1..25: note = morpholino antisense oligonucleotide
  source 1..25: mol_type = other DNA
agtaaccacaggttgtgtcaccaga

SEQ ID NO: 246 - DNA, 25 bp, synthetic construct
  misc_feature 1..25: note = morpholino antisense oligonucleotide
  source 1..25: mol_type = other DNA
ccttagtaaccacaggttgtgtcac

SEQ ID NO: 247 - DNA, 25 bp, synthetic construct
  misc_feature 1..25: note = morpholino antisense oligonucleotide
  source 1..25: mol_type = other DNA
gtgtcaccagagtaacagtctgagt

SEQ ID NO: 248 - DNA, 25 bp, synthetic construct
  misc_feature 1..25: note = morpholino antisense oligonucleotide
  source 1..25: mol_type = other DNA
caccagagtaacagtctgagtagga

SEQ ID NO: 249 - DNA, 25 bp, synthetic construct
  misc_feature 1..25: note = morpholino antisense oligonucleotide
  source 1..25: mol_type = other DNA
tgtgtcaccagagtaacagtctgag

SEQ ID NO: 250 - DNA, 25 bp, synthetic construct
  misc_feature 1..25: note = morpholino antisense oligonucleotide
  source 1..25: mol_type = other DNA
tccttagtaaccacaggttgtgtca

SEQ ID NO: 251 - DNA, 25 bp, synthetic construct
  misc_feature 1..25: note = morpholino antisense oligonucleotide
  source 1..25: mol_type = other DNA
tcaccagagtaacagtctgagtagg

SEQ ID NO: 252 - DNA, 25 bp, synthetic construct
  misc_feature 1..25: note = morpholino antisense oligonucleotide
  source 1..25: mol_type = other DNA
gtcaccagagtaacagtctgagtag

SEQ ID NO: 253 - DNA, 25 bp, synthetic construct
  misc_feature 1..25: note = morpholino antisense oligonucleotide
  source 1..25: mol_type = other DNA
ttccttagtaaccacaggttgtgtc

SEQ ID NO: 254 - DNA, 25 bp, synthetic construct
  misc_feature 1..25: note = morpholino antisense oligonucleotide
  source 1..25: mol_type = other DNA
gtttccttagtaaccacaggttgtg

SEQ ID NO: 255 - DNA, 25 bp, synthetic construct
  misc_feature 1..25: note = morpholino antisense oligonucleotide
  source 1..25: mol_type = other DNA
cagtttccttagtaaccacaggttg

SEQ ID NO: 256 - DNA, 25 bp, synthetic construct
  misc_feature 1..25: note = morpholino antisense oligonucleotide
  source 1..25: mol_type = other DNA
gatggcagtttccttagtaaccaca

SEQ ID NO: 257 - DNA, 25 bp, synthetic construct
  misc_feature 1..25: note = morpholino antisense oligonucleotide
  source 1..25: mol_type = other DNA
gttgtgtcaccagagtaacagtctg

SEQ ID NO: 258 - DNA, 25 bp, synthetic construct
  misc_feature 1..25: note = morpholino antisense oligonucleotide
  source 1..25: mol_type = other DNA
ttgtgtcaccagagtaacagtctga

SEQ ID NO: 259 - DNA, 25 bp, synthetic construct
  misc_feature 1..25: note = morpholino antisense oligonucleotide
  source 1..25: mol_type = other DNA
tgtcaccagagtaacagtctgagta

SEQ ID NO: 260 - DNA, 25 bp, synthetic construct
  misc_feature 1..25: note = morpholino antisense oligonucleotide
  source 1..25: mol_type = other DNA
tttccttagtaaccacaggttgtgt

SEQ ID NO: 261 - DNA, 25 bp, synthetic construct
  misc_feature 1..25: note = morpholino antisense oligonucleotide
  source 1..25: mol_type = other DNA
cttagtaaccacaggttgtgtcacc

SEQ ID NO: 262 - DNA, 25 bp, synthetic construct
  misc_feature 1..25: note = morpholino antisense oligonucleotide
  source 1..25: mol_type = other DNA
atggcagtttccttagtaaccacag

SEQ ID NO: 263 - DNA, 25 bp, synthetic construct
  misc_feature 1..25: note = morpholino antisense oligonucleotide
  source 1..25: mol_type = other DNA
tctgtccaagcccggttgaaatctg

SEQ ID NO: 264 - DNA, 25 bp, synthetic construct
  misc_feature 1..25: note = morpholino antisense oligonucleotide
  source 1..25: mol_type = other DNA
tctgccagagcaggtacctccaaca

SEQ ID NO: 265 - DNA, 25 bp, synthetic construct
  misc_feature 1..25: note = morpholino antisense oligonucleotide
  source 1..25: mol_type = other DNA
cccggttgaaatctgccagagcagg

SEQ ID NO: 266 - DNA, 25 bp, synthetic construct
  misc_feature 1..25: note = morpholino antisense oligonucleotide
  source 1..25: mol_type = other DNA
agtttccttagtaaccacaggttgt

SEQ ID NO: 267 - DNA, 25 bp, synthetic construct
  misc_feature 1..25: note = morpholino antisense oligonucleotide
  source 1..25: mol_type = other DNA
agatggcagtttccttagtaaccac

SEQ ID NO: 268 - DNA, 25 bp, synthetic construct
  misc_feature 1..25: note = morpholino antisense oligonucleotide
  source 1..25: mol_type = other DNA
tagtaaccacaggttgtgtcaccag

SEQ ID NO: 269 - DNA, 25 bp, synthetic construct
  misc_feature 1..25: note = morpholino antisense oligonucleotide
  source 1..25: mol_type = other DNA
ccagagcaggtacctccaacatcaa

SEQ ID NO: 270 - DNA, 25 bp, synthetic construct
  misc_feature 1..25: note = morpholino antisense oligonucleotide
  source 1..25: mol_type = other DNA
ctgccagagcaggtacctccaacat

SEQ ID NO: 271 - DNA, 25 bp, synthetic construct
  misc_feature 1..25: note = morpholino antisense oligonucleotide
  source 1..25: mol_type = other DNA
cagagcaggtacctccaacatcaag

SEQ ID NO: 272 - DNA, 25 bp, synthetic construct
  misc_feature 1..25: note = morpholino antisense oligonucleotide
  source 1..25: mol_type = other DNA
gccagagcaggtacctccaacatca

SEQ ID NO: 273 - DNA, 25 bp, synthetic construct
  misc_feature 1..25: note = morpholino antisense oligonucleotide
  source 1..25: mol_type = other DNA
tgccagagcaggtacctccaacatc

SEQ ID NO: 274 - DNA, 25 bp, synthetic construct
  misc_feature 1..25: note = morpholino antisense oligonucleotide
  source 1..25: mol_type = other DNA
gttctgtccaagcccggttgaaatc

SEQ ID NO: 275 - DNA, 25 bp, synthetic construct
  misc_feature 1..25: note = morpholino antisense oligonucleotide
  source 1..25: mol_type = other DNA
ctgtccaagcccggttgaaatctgc

SEQ ID NO: 276 - DNA, 25 bp, synthetic construct
  misc_feature 1..25: note = morpholino antisense oligonucleotide
  source 1..25: mol_type = other DNA
ttctgtccaagcccggttgaaatct

SEQ ID NO: 277 - DNA, 25 bp, synthetic construct
  misc_feature 1..25: note = morpholino antisense oligonucleotide
  source 1..25: mol_type = other DNA
gcccggttgaaatctgccagagcag

SEQ ID NO: 278 - DNA, 25 bp, synthetic construct
  misc_feature 1..25: note = morpholino antisense oligonucleotide
  source 1..25: mol_type = other DNA
agagcaggtacctccaacatcaagg

SEQ ID NO: 279 - DNA, 25 bp, synthetic construct
  misc_feature 1..25: note = morpholino antisense oligonucleotide
  source 1..25: mol_type = other DNA
gagatggcagtttccttagtaacca

SEQ ID NO: 280 - DNA, 25 bp, synthetic construct
  misc_feature 1..25: note = morpholino antisense oligonucleotide
  source 1..25: mol_type = other DNA
ggcatttctagtttggagatggcag

SEQ ID NO: 281 - DNA, 25 bp, synthetic construct
  misc_feature 1..25: note = morpholino antisense oligonucleotide
  source 1..25: mol_type = other DNA
agttctgtccaagcccggttgaaat

SEQ ID NO: 282 - DNA, 25 bp, synthetic construct
  misc_feature 1..25: note = morpholino antisense oligonucleotide
  source 1..25: mol_type = other DNA
ttagtaaccacaggttgtgtcacca

SEQ ID NO: 283 - DNA, 25 bp, synthetic construct
  misc_feature 1..25: note = morpholino antisense oligonucleotide
  source 1..25: mol_type = other DNA
tgtccaagcccggttgaaatctgcc

SEQ ID NO: 284 - DNA, 25 bp, synthetic construct
  misc_feature 1..25: note = morpholino antisense oligonucleotide
  source 1..25: mol_type = other DNA
aagttctgtccaagcccggttgaaa

SEQ ID NO: 285 - DNA, 25 bp, synthetic construct
  misc_feature 1..25: note = morpholino antisense oligonucleotide
  source 1..25: mol_type = other DNA
ggtaagttctgtccaagcccggttg

SEQ ID NO: 286 - DNA, 25 bp, synthetic construct
  misc_feature 1..25: note = morpholino antisense oligonucleotide
  source 1..25: mol_type = other DNA
gcatttctagtttggagatggcagt

SEQ ID NO: 287 - DNA, 25 bp, synthetic construct
  misc_feature 1..25: note = morpholino antisense oligonucleotide
  source 1..25: mol_type = other DNA
aagcccggttgaaatctgccagagc

SEQ ID NO: 288 - DNA, 25 bp, synthetic construct
  misc_feature 1..25: note = morpholino antisense oligonucleotide
  source 1..25: mol_type = other DNA
ccaagcccggttgaaatctgccaga

SEQ ID NO: 289 - DNA, 25 bp, synthetic construct
  misc_feature 1..25: note = morpholino antisense oligonucleotide
  source 1..25: mol_type = other DNA
taagttctgtccaagcccggttgaa

SEQ ID NO: 290 - DNA, 25 bp, synthetic construct
  misc_feature 1..25: note = morpholino antisense oligonucleotide
  source 1..25: mol_type = other DNA
gtccaagcccggttgaaatctgcca

SEQ ID NO: 291 - DNA, 25 bp, synthetic construct
  misc_feature 1..25: note = morpholino antisense oligonucleotide
  source 1..25: mol_type = other DNA
atggcatttctagtttggagatggc

SEQ ID NO: 292 - DNA, 25 bp, synthetic construct
  misc_feature 1..25: note = morpholino antisense oligonucleotide
  source 1..25: mol_type = other DNA
agcccggttgaaatctgccagagca

SEQ ID NO: 293 - DNA, 25 bp, synthetic construct
  misc_feature 1..25: note = morpholino antisense oligonucleotide
  source 1..25: mol_type = other DNA
cggtaagttctgtccaagcccggtt

SEQ ID NO: 294 - DNA, 25 bp, synthetic construct
  misc_feature 1..25: note = morpholino antisense oligonucleotide
  source 1..25: mol_type = other DNA
ccggttgaaatctgccagagcaggt

SEQ ID NO: 295 - DNA, 25 bp, synthetic construct
  misc_feature 1..25: note = morpholino antisense oligonucleotide
  source 1..25: mol_type = other DNA
tcggtaagttctgtccaagcccggt

SEQ ID NO: 296 - DNA, 25 bp, synthetic construct
  misc_feature 1..25: note = morpholino antisense oligonucleotide
  source 1..25: mol_type = other DNA
caagcccggttgaaatctgccagag

SEQ ID NO: 297 - DNA, 25 bp, synthetic construct
  misc_feature 1..25: note = morpholino antisense oligonucleotide
  source 1..25: mol_type = other DNA
gtaagttctgtccaagcccggttga

SEQ ID NO: 298 - DNA, 25 bp, synthetic construct
  misc_feature 1..25: note = morpholino antisense oligonucleotide
  source 1..25: mol_type = other DNA
tccaagcccggttgaaatctgccag

SEQ ID NO: 299 - DNA, 25 bp, synthetic construct
  misc_feature 1..25: note = morpholino antisense oligonucleotide
  source 1..25: mol_type = other DNA
gaaatctgccagagcaggtacctcc

SEQ ID NO: 300 - DNA, 25 bp, synthetic construct
  misc_feature 1..25: note = morpholino antisense oligonucleotide
  source 1..25: mol_type = other DNA
ggagatggcagtttccttagtaacc

SEQ ID NO: 301 - DNA, 25 bp, synthetic construct
  misc_feature 1..25: note = morpholino antisense oligonucleotide
  source 1..25: mol_type = other DNA
atctgccagagcaggtacctccaac

SEQ ID NO: 302 - DNA, 25 bp, synthetic construct
  misc_feature 1..25: note = morpholino antisense oligonucleotide
  source 1..25: mol_type = other DNA
gtcggtaagttctgtccaagcccgg

SEQ ID NO: 303 - DNA, 25 bp, synthetic construct
  misc_feature 1..25: note = morpholino antisense oligonucleotide
  source 1..25: mol_type = other DNA
ggttgaaatctgccagagcaggtac

SEQ ID NO: 304 - DNA, 25 bp, synthetic construct
  misc_feature 1..25: note = morpholino antisense oligonucleotide
  source 1..25: mol_type = other DNA
gaagatggcatttctagtttggaga

SEQ ID NO: 305 - DNA, 25 bp, synthetic construct
  misc_feature 1..25: note = morpholino antisense oligonucleotide
  source 1..25: mol_type = other DNA
ttctagtttggagatggcagtttcc

SEQ ID NO: 306 - DNA, 25 bp, synthetic construct
  misc_feature 1..25: note = morpholino antisense oligonucleotide
  source 1..25: mol_type = other DNA
tttctagtttggagatggcagtttc

SEQ ID NO: 307 - DNA, 25 bp, synthetic construct
  misc_feature 1..25: note = morpholino antisense oligonucleotide
  source 1..25: mol_type = other DNA
aatctgccagagcaggtacctccaa

SEQ ID NO: 308 - DNA, 25 bp, synthetic construct
  misc_feature 1..25: note = morpholino antisense oligonucleotide
  source 1..25: mol_type = other DNA
ggaagatggcatttctagtttggag

SEQ ID NO: 309 - DNA, 25 bp, synthetic construct
  misc_feature 1..25: note = morpholino antisense oligonucleotide
  source 1..25: mol_type = other DNA
cggttgaaatctgccagagcaggta

SEQ ID NO: 310 - DNA, 25 bp, synthetic construct
  misc_feature 1..25: note = morpholino antisense oligonucleotide
  source 1..25: mol_type = other DNA
gcagagaaagccagtcggtaagttc

SEQ ID NO: 311 - DNA, 25 bp, synthetic construct
  misc_feature 1..25: note = morpholino antisense oligonucleotide
  source 1..25: mol_type = other DNA
gagcaggtacctccaacatcaagga

SEQ ID NO: 312 - DNA, 25 bp, synthetic construct
  misc_feature 1..25: note = morpholino antisense oligonucleotide
  source 1..25: mol_type = other DNA
cagtcggtaagttctgtccaagccc

SEQ ID NO: 313 - DNA, 25 bp, synthetic construct
  misc_feature 1..25: note = morpholino antisense oligonucleotide
  source 1..25: mol_type = other DNA
gagaaagccagtcggtaagttctgt

SEQ ID NO: 314 - DNA, 25 bp, synthetic construct
  misc_feature 1..25: note = morpholino antisense oligonucleotide
  source 1..25: mol_type = other DNA
agtcggtaagttctgtccaagcccg

SEQ ID NO: 315 - DNA, 25 bp, synthetic construct
  misc_feature 1..25: note = morpholino antisense oligonucleotide
  source 1..25: mol_type = other DNA
tgaaatctgccagagcaggtacctc

SEQ ID NO: 316 - DNA, 25 bp, synthetic construct
  misc_feature 1..25: note = morpholino antisense oligonucleotide
  source 1..25: mol_type = other DNA
tttggagatggcagtttccttagta

SEQ ID NO: 317 - DNA, 25 bp, synthetic construct
  misc_feature 1..25: note = morpholino antisense oligonucleotide
  source 1..25: mol_type = other DNA
aaatctgccagagcaggtacctcca

SEQ ID NO: 318 - DNA, 25 bp, synthetic construct
  misc_feature 1..25: note = morpholino antisense oligonucleotide
  source 1..25: mol_type = other DNA
agagaaagccagtcggtaagttctg

SEQ ID NO: 319 - DNA, 25 bp, synthetic construct
  misc_feature 1..25: note = morpholino antisense oligonucleotide
  source 1..25: mol_type = other DNA
ccagtcggtaagttctgtccaagcc

SEQ ID NO: 320 - DNA, 25 bp, synthetic construct
  misc_feature 1..25: note = morpholino antisense oligonucleotide
  source 1..25: mol_type = other DNA
gccagtcggtaagttctgtccaagc

SEQ ID NO: 321 - DNA, 25 bp, synthetic construct
  misc_feature 1..25: note = morpholino antisense oligonucleotide
  source 1..25: mol_type = other DNA
tggagatggcagtttccttagtaac

SEQ ID NO: 322 - DNA, 25 bp, synthetic construct
  misc_feature 1..25: note = morpholino antisense oligonucleotide
  source 1..25: mol_type = other DNA
tctagtttggagatggcagtttcct

SEQ ID NO: 323 - DNA, 25 bp, synthetic construct
  misc_feature 1..25: note = morpholino antisense oligonucleotide
  source 1..25: mol_type = other DNA
gcaggtacctccaacatcaaggaag

SEQ ID NO: 324 - DNA, 25 bp, synthetic construct
  misc_feature 1..25: note = morpholino antisense oligonucleotide
  source 1..25: mol_type = other DNA
atttctagtttggagatggcagttt

SEQ ID NO: 325 - DNA, 25 bp, synthetic construct
  misc_feature 1..25: note = morpholino antisense oligonucleotide
  source 1..25: mol_type = other DNA
aaagccagtcggtaagttctgtcca

SEQ ID NO: 326 - DNA, 25 bp, synthetic construct
  misc_feature 1..25: note = morpholino antisense oligonucleotide
  source 1..25: mol_type = other DNA
gaaagccagtcggtaagttctgtcc

SEQ ID NO: 327 - DNA, 25 bp, synthetic construct
  misc_feature 1..25: note = morpholino antisense oligonucleotide
  source 1..25: mol_type = other DNA
ttggagatggcagtttccttagtaa

SEQ ID NO: 328 - DNA, 25 bp, synthetic construct
  misc_feature 1..25: note = morpholino antisense oligonucleotide
  source 1..25: mol_type = other DNA
caagcagagaaagccagtcggtaag

SEQ ID NO: 329 - DNA, 25 bp, synthetic construct
  misc_feature 1..25: note = morpholino antisense oligonucleotide
  source 1..25: mol_type = other DNA
agcagagaaagccagtcggtaagtt

SEQ ID NO: 330 - DNA, 25 bp, synthetic construct
  misc_feature 1..25: note = morpholino antisense oligonucleotide
  source 1..25: mol_type = other DNA
gatcaagcagagaaagccagtcggt

SEQ ID NO: 331 - DNA, 25 bp, synthetic construct
  misc_feature 1..25: note = morpholino antisense oligonucleotide
  source 1..25: mol_type = other DNA
aagcagagaaagccagtcggtaagt

SEQ ID NO: 332 - DNA, 25 bp, synthetic construct
  misc_feature 1..25: note = morpholino antisense oligonucleotide
  source 1..25: mol_type = other DNA
tgatcaagcagagaaagccagtcgg

SEQ ID NO: 333 - DNA, 25 bp, synthetic construct
  misc_feature 1..25: note = morpholino antisense oligonucleotide
  source 1..25: mol_type = other DNA
agaaagccagtcggtaagttctgtc

SEQ ID NO: 334 - DNA, 25 bp, synthetic construct
  misc_feature 1..25: note = morpholino antisense oligonucleotide
  source 1..25: mol_type = other DNA
atcaagcagagaaagccagtcggta

SEQ ID NO: 335 - DNA, 25 bp, synthetic construct
  misc_feature 1..25: note = morpholino antisense oligonucleotide
  source 1..25: mol_type = other DNA
acctccaacatcaaggaagatggca

SEQ ID NO: 336 - DNA, 25 bp, synthetic construct
  misc_feature 1..25: note = morpholino antisense oligonucleotide
  source 1..25: mol_type = other DNA
agcaggtacctccaacatcaaggaa

SEQ ID NO: 337 - DNA, 25 bp, synthetic construct
  misc_feature 1..25: note = morpholino antisense oligonucleotide
  source 1..25: mol_type = other DNA
cagagaaagccagtcggtaagttct

SEQ ID NO: 338 - DNA, 25 bp, synthetic construct
  misc_feature 1..25: note = morpholino antisense oligonucleotide
  source 1..25: mol_type = other DNA
gttgaaatctgccagagcaggtacc

SEQ ID NO: 339 - DNA, 25 bp, synthetic construct
  misc_feature 1..25: note = morpholino antisense oligonucleotide
  source 1..25: mol_type = other DNA
aggaagatggcatttctagtttgga

SEQ ID NO: 340 - DNA, 25 bp, synthetic construct
  misc_feature 1..25: note = morpholino antisense oligonucleotide
  source 1..25: mol_type = other DNA
aagatggcatttctagtttggagat

SEQ ID NO: 341 - DNA, 25 bp, synthetic construct
  misc_feature 1..25: note = morpholino antisense oligonucleotide
  source 1..25: mol_type = other DNA
ttgaaatctgccagagcaggtacct

SEQ ID NO: 342 - DNA, 25 bp, synthetic construct
  misc_feature 1..25: note = morpholino antisense oligonucleotide
  source 1..25: mol_type = other DNA
cttgatcaagcagagaaagccagtc

SEQ ID NO: 343 - DNA, 25 bp, synthetic construct
  misc_feature 1..25: note = morpholino antisense oligonucleotide
  source 1..25: mol_type = other DNA
ctccaacatcaaggaagatggcatt

SEQ ID NO: 344 - DNA, 25 bp, synthetic construct
  misc_feature 1..25: note = morpholino antisense oligonucleotide
  source 1..25: mol_type = other DNA
cctccaacatcaaggaagatggcat

SEQ ID NO: 345 - DNA, 25 bp, synthetic construct
  misc_feature 1..25: note = morpholino antisense oligonucleotide
  source 1..25: mol_type = other DNA
tggcatttctagtttggagatggca

SEQ ID NO: 346 - DNA, 25 bp, synthetic construct
  misc_feature 1..25: note = morpholino antisense oligonucleotide
  source 1..25: mol_type = other DNA
atcaaggaagatggcatttctagtt

SEQ ID NO: 347 - DNA, 25 bp, synthetic construct
  misc_feature 1..25: note = morpholino antisense oligonucleotide
  source 1..25: mol_type = other DNA
agtttggagatggcagtttccttag

SEQ ID NO: 348 - DNA, 25 bp, synthetic construct
  misc_feature 1..25: note = morpholino antisense oligonucleotide
  source 1..25: mol_type = other DNA
gatggcatttctagtttggagatgg

SEQ ID NO: 349 - DNA, 25 bp, synthetic construct
  misc_feature 1..25: note = morpholino antisense oligonucleotide
  source 1..25: mol_type = other DNA
catttctagtttggagatggcagtt

SEQ ID NO: 350 - DNA, 25 bp, synthetic construct
  misc_feature 1..25: note = morpholino antisense oligonucleotide
  source 1..25: mol_type = other DNA
tcaagcagagaaagccagtcggtaa

SEQ ID NO: 351 - DNA, 25 bp, synthetic construct
  misc_feature 1..25: note = morpholino antisense oligonucleotide
  source 1..25: mol_type = other DNA
agccagtcggtaagttctgtccaag

SEQ ID NO: 352 - DNA, 25 bp, synthetic construct
  misc_feature 1..25: note = morpholino antisense oligonucleotide
  source 1..25: mol_type = other DNA
acttgatcaagcagagaaagccagt

SEQ ID NO: 353 - DNA, 25 bp, synthetic construct
  misc_feature 1..25: note = morpholino antisense oligonucleotide
  source 1..25: mol_type = other DNA
ccaacatcaaggaagatggcatttc

SEQ ID NO: 354 - DNA, 25 bp, synthetic construct
  misc_feature 1..25: note = morpholino antisense oligonucleotide
  source 1..25: mol_type = other DNA
aagccagtcggtaagttctgtccaa

SEQ ID NO: 355 - DNA, 25 bp, synthetic construct
  misc_feature 1..25: note = morpholino antisense oligonucleotide
  source 1..25: mol_type = other DNA
aaggaagatggcatttctagtttgg

SEQ ID NO: 356 - DNA, 25 bp, synthetic construct
  misc_feature 1..25: note = morpholino antisense oligonucleotide
  source 1..25: mol_type = other DNA
agatggcatttctagtttggagatg

SEQ ID NO: 357 - DNA, 25 bp, synthetic construct
  misc_feature 1..25: note = morpholino antisense oligonucleotide
  source 1..25: mol_type = other DNA
aacttgatcaagcagagaaagccag

SEQ ID NO: 358 - DNA, 25 bp, synthetic construct
  misc_feature 1..25: note = morpholino antisense oligonucleotide
  source 1..25: mol_type = other DNA
tacctccaacatcaaggaagatggc

SEQ ID NO: 359 - DNA, 25 bp, synthetic construct
  misc_feature 1..25: note = morpholino antisense oligonucleotide
  source 1..25: mol_type = other DNA
ttgatcaagcagagaaagccagtcg

SEQ ID NO: 360 - DNA, 25 bp, synthetic construct
  misc_feature 1..25: note = morpholino antisense oligonucleotide
  source 1..25: mol_type = other DNA
gtttggagatggcagtttccttagt

SEQ ID NO: 361 - DNA, 25 bp, synthetic construct
  misc_feature 1..25: note = morpholino antisense oligonucleotide
  source 1..25: mol_type = other DNA
gtacctccaacatcaaggaagatgg

SEQ ID NO: 362 - DNA, 25 bp, synthetic construct
  misc_feature 1..25: note = morpholino antisense oligonucleotide
  source 1..25: mol_type = other DNA
catcaaggaagatggcatttctagt

SEQ ID NO: 363 - DNA, 25 bp, synthetic construct
  misc_feature 1..25: note = morpholino antisense oligonucleotide
  source 1..25: mol_type = other DNA
acatcaaggaagatggcatttctag

SEQ ID NO: 364 - DNA, 25 bp, synthetic construct
  misc_feature 1..25: note = morpholino antisense oligonucleotide
  source 1..25: mol_type = other DNA
ctagtttggagatggcagtttcctt

SEQ ID NO: 365 - DNA, 25 bp, synthetic construct
  misc_feature 1..25: note = morpholino antisense oligonucleotide
  source 1..25: mol_type = other DNA
caaggaagatggcatttctagtttg

SEQ ID NO: 366 - DNA, 25 bp, synthetic construct
  misc_feature 1..25: note = morpholino antisense oligonucleotide
  source 1..25: mol_type = other DNA
gtcacccaccatcaccctctgtgat

SEQ ID NO: 367 - DNA, 25 bp, synthetic construct
  misc_feature 1..25: note = morpholino antisense oligonucleotide
  source 1..25: mol_type = other DNA
tccaacatcaaggaagatggcattt

SEQ ID NO: 368 - DNA, 25 bp, synthetic construct
  misc_feature 1..25: note = morpholino antisense oligonucleotide
  source 1..25: mol_type = other DNA
taacttgatcaagcagagaaagcca

SEQ ID NO: 369 - DNA, 25 bp, synthetic construct
  misc_feature 1..25: note = morpholino antisense oligonucleotide
  source 1..25: mol_type = other DNA
tcaaggaagatggcatttctagttt

SEQ ID NO: 370 - DNA, 25 bp, synthetic construct
  misc_feature 1..25: note = morpholino antisense oligonucleotide
  source 1..25: mol_type = other DNA
caggtacctccaacatcaaggaaga

SEQ ID NO: 371 - DNA, 25 bp, synthetic construct
  misc_feature 1..25: note = morpholino antisense oligonucleotide
  source 1..25: mol_type = other DNA
caacatcaaggaagatggcatttct

SEQ ID NO: 372 - DNA, 25 bp, synthetic construct
  misc_feature 1..25: note = morpholino antisense oligonucleotide
  source 1..25: mol_type = other DNA
ggtcacccaccatcaccctctgtga

SEQ ID NO: 373 - DNA, 25 bp, synthetic construct
  misc_feature 1..25: note = morpholino antisense oligonucleotide
  source 1..25: mol_type = other DNA
ataacttgatcaagcagagaaagcc

SEQ ID NO: 374 - DNA, 25 bp, synthetic construct
  misc_feature 1..25: note = morpholino antisense oligonucleotide
  source 1..25: mol_type = other DNA
ggtacctccaacatcaaggaagatg

SEQ ID NO: 375 - DNA, 25 bp, synthetic construct
  misc_feature 1..25: note = morpholino antisense oligonucleotide
  source 1..25: mol_type = other DNA
aggtcacccaccatcaccctctgtg

SEQ ID NO: 376 - DNA, 25 bp, synthetic construct
  misc_feature 1..25: note = morpholino antisense oligonucleotide
  source 1..25: mol_type = other DNA
aacatcaaggaagatggcatttcta

SEQ ID NO: 377 - DNA, 25 bp, synthetic construct
  misc_feature 1..25: note = morpholino antisense oligonucleotide
  source 1..25: mol_type = other DNA
tagtttggagatggcagtttcctta

SEQ ID NO: 378 - DNA, 25 bp, synthetic construct
  misc_feature 1..25: note = morpholino antisense oligonucleotide
  source 1..25: mol_type = other DNA
ttataacttgatcaagcagagaaag

SEQ ID NO: 379 - DNA, 25 bp, synthetic construct
  misc_feature 1..25: note = morpholino antisense oligonucleotide
  source 1..25: mol_type = other DNA
tcacccaccatcaccctctgtgatt

SEQ ID NO: 380 - DNA, 25 bp, synthetic construct
  misc_feature 1..25: note = morpholino antisense oligonucleotide
  source 1..25: mol_type = other DNA
cacccaccatcaccctctgtgattt

SEQ ID NO: 381 - DNA, 25 bp, synthetic construct
  misc_feature 1..25: note = morpholino antisense oligonucleotide
  source 1..25: mol_type = other DNA
tttataacttgatcaagcagagaaa

SEQ ID NO: 382 - DNA, 25 bp, synthetic construct
  misc_feature 1..25: note = morpholino antisense oligonucleotide
  source 1..25: mol_type = other DNA
caaggtcacccaccatcaccctctg

SEQ ID NO: 383 - DNA, 25 bp, synthetic construct
  misc_feature 1..25: note = morpholino antisense oligonucleotide
  source 1..25: mol_type = other DNA
tataacttgatcaagcagagaaagc

SEQ ID NO: 384 - DNA, 25 bp, synthetic construct
  misc_feature 1..25: note = morpholino antisense oligonucleotide
  source 1..25: mol_type = other DNA
cccaccatcaccctctgtgatttta

SEQ ID NO: 385 - DNA, 25 bp, synthetic construct
  misc_feature 1..25: note = morpholino antisense oligonucleotide
  source 1..25: mol_type = other DNA
acccaccatcaccctctgtgatttt

SEQ ID NO: 386 - DNA, 25 bp, synthetic construct
  misc_feature 1..25: note = morpholino antisense oligonucleotide
  source 1..25: mol_type = other DNA
tcaaggtcacccaccatcaccctct

SEQ ID NO: 387 - DNA, 25 bp, synthetic construct
  misc_feature 1..25: note = morpholino antisense oligonucleotide
  source 1..25: mol_type = other DNA
atcctcaaggtcacccaccatcacc

SEQ ID NO: 388 - DNA, 25 bp, synthetic construct
  misc_feature 1..25: note = morpholino antisense oligonucleotide
  source 1..25: mol_type = other DNA
aaggtcacccaccatcaccctctgt

SEQ ID NO: 389 - DNA, 25 bp, synthetic construct
  misc_feature 1..25: note = morpholino antisense oligonucleotide
  source 1..25: mol_type = other DNA
tcctcaaggtcacccaccatcaccc

SEQ ID NO: 390 - DNA, 25 bp, synthetic construct
  misc_feature 1..25: note = morpholino antisense oligonucleotide
  source 1..25: mol_type = other DNA
cctcaaggtcacccaccatcaccct

SEQ ID NO: 391 - DNA, 25 bp, synthetic construct
  misc_feature 1..25: note = morpholino antisense oligonucleotide
  source 1..25: mol_type = other DNA
atatcctcaaggtcacccaccatca

SEQ ID NO: 392 - DNA, 25 bp, synthetic construct
  misc_feature 1..25: note = morpholino antisense oligonucleotide
  source 1..25: mol_type = other DNA
ctcaaggtcacccaccatcaccctc

SEQ ID NO: 393 - DNA, 25 bp, synthetic construct
  misc_feature 1..25: note = morpholino antisense oligonucleotide
  source 1..25: mol_type = other DNA
aggtacctccaacatcaaggaagat

SEQ ID NO: 394 - DNA, 25 bp, synthetic construct
  misc_feature 1..25: note = morpholino antisense oligonucleotide
  source 1..25: mol_type = other DNA
tatcctcaaggtcacccaccatcac

SEQ ID NO: 395 - DNA, 25 bp, synthetic construct
  misc_feature 1..25: note = morpholino antisense oligonucleotide
  source 1..25: mol_type = other DNA
ctctgtgattttataacttgatcaa

SEQ ID NO: 396 - DNA, 25 bp, synthetic construct
  misc_feature 1..25: note = morpholino antisense oligonucleotide
  source 1..25: mol_type = other DNA
ccaccatcaccctctgtgattttat

SEQ ID NO: 397 - DNA, 25 bp, synthetic construct
  misc_feature 1..25: note = morpholino antisense oligonucleotide
  source 1..25: mol_type = other DNA
cctctgtgattttataacttgatca

SEQ ID NO: 398 - DNA, 25 bp, synthetic construct
  misc_feature 1..25: note = morpholino antisense oligonucleotide
  source 1..25: mol_type = other DNA
gatatcctcaaggtcacccaccatc

SEQ ID NO: 399 - DNA, 25 bp, synthetic construct
  misc_feature 1..25: note = morpholino antisense oligonucleotide
  source 1..25: mol_type = other DNA
ttttataacttgatcaagcagagaa

SEQ ID NO: 400 - DNA, 25 bp, synthetic construct
  misc_feature 1..25: note = morpholino antisense oligonucleotide
  source 1..25: mol_type = other DNA
ctcgttgatatcctcaaggtcaccc

SEQ ID NO: 401 - DNA, 25 bp, synthetic construct
  misc_feature 1..25: note = morpholino antisense oligonucleotide
  source 1..25: mol_type = other DNA
tcgttgatatcctcaaggtcaccca

SEQ ID NO: 402 - DNA, 25 bp, synthetic construct
  misc_feature 1..25: note = morpholino antisense oligonucleotide
  source 1..25: mol_type = other DNA
tgatatcctcaaggtcacccaccat

SEQ ID NO: 403 - DNA, 25 bp, synthetic construct
  misc_feature 1..25: note = morpholino antisense oligonucleotide
  source 1..25: mol_type = other DNA
cgttgatatcctcaaggtcacccac

SEQ ID NO: 404 - DNA, 25 bp, synthetic construct
  misc_feature 1..25: note = morpholino antisense oligonucleotide
  source 1..25: mol_type = other DNA
attttataacttgatcaagcagaga

SEQ ID NO: 405 - DNA, 25 bp, synthetic construct
  misc_feature 1..25: note = morpholino antisense oligonucleotide
  source 1..25: mol_type = other DNA
tctcgttgatatcctcaaggtcacc

SEQ ID NO: 406 - DNA, 25 bp, synthetic construct
  misc_feature 1..25: note = morpholino antisense oligonucleotide
  source 1..25: mol_type = other DNA
caccctctgtgattttataacttga

SEQ ID NO: 407 - DNA, 25 bp, synthetic construct
  misc_feature 1..25: note = morpholino antisense oligonucleotide
  source 1..25: mol_type = other DNA
gttgatatcctcaaggtcacccacc

SEQ ID NO: 408 - DNA, 25 bp, synthetic construct
  misc_feature 1..25: note = morpholino antisense oligonucleotide
  source 1..25: mol_type = other DNA
tctgtgattttataacttgatcaag

SEQ ID NO: 409 - DNA, 25 bp, synthetic construct
  misc_feature 1..25: note = morpholino antisense oligonucleotide
  source 1..25: mol_type = other DNA
ccctctgtgattttataacttgatc

SEQ ID NO: 410 - DNA, 25 bp, synthetic construct
  misc_feature 1..25: note = morpholino antisense oligonucleotide
  source 1..25: mol_type = other DNA
atctcgttgatatcctcaaggtcac

SEQ ID NO: 411 - DNA, 25 bp, synthetic construct
  misc_feature 1..25: note = morpholino antisense oligonucleotide
  source 1..25: mol_type = other DNA
ttgatatcctcaaggtcacccacca

SEQ ID NO: 412 - DNA, 25 bp, synthetic construct
  misc_feature 1..25: note = morpholino antisense oligonucleotide
  source 1..25: mol_type = other DNA
catctcgttgatatcctcaaggtca

SEQ ID NO: 413 - DNA, 25 bp, synthetic construct
  misc_feature 1..25: note = morpholino antisense oligonucleotide
  source 1..25: mol_type = other DNA
tcaccctctgtgattttataacttg

SEQ ID NO: 414 - DNA, 25 bp, synthetic construct
  misc_feature 1..25: note = morpholino antisense oligonucleotide
  source 1..25: mol_type = other DNA
caccatcaccctctgtgattttata

SEQ ID NO: 415 - DNA, 25 bp, synthetic construct
  misc_feature 1..25: note = morpholino antisense oligonucleotide
  source 1..25: mol_type = other DNA
tcatctcgttgatatcctcaaggtc

SEQ ID NO: 416 - DNA, 25 bp, synthetic construct
  misc_feature 1..25: note = morpholino antisense oligonucleotide
  source 1..25: mol_type = other DNA
gatcatctcgttgatatcctcaagg

SEQ ID NO: 417 - DNA, 25 bp, synthetic construct
  misc_feature 1..25: note = morpholino antisense oligonucleotide
  source 1..25: mol_type = other DNA
gattttataacttgatcaagcagag

SEQ ID NO: 418 - DNA, 25 bp, synthetic construct
  misc_feature 1..25: note = morpholino antisense oligonucleotide
  source 1..25: mol_type = other DNA
tgatcatctcgttgatatcctcaag

SEQ ID NO: 419 - DNA, 25 bp, synthetic construct
  misc_feature 1..25: note = morpholino antisense oligonucleotide
  source 1..25: mol_type = other DNA
gatgatcatctcgttgatatcctca

SEQ ID NO: 420 - DNA, 25 bp, synthetic construct
  misc_feature 1..25: note = morpholino antisense oligonucleotide
  source 1..25: mol_type = other DNA
atgatcatctcgttgatatcctcaa

SEQ ID NO: 421 - DNA, 25 bp, synthetic construct
  misc_feature 1..25: note = morpholino antisense oligonucleotide
  source 1..25: mol_type = other DNA
tgatgatcatctcgttgatatcctc

SEQ ID NO: 422 - DNA, 25 bp, synthetic construct
  misc_feature 1..25: note = morpholino antisense oligonucleotide
  source 1..25: mol_type = other DNA
ttgatgatcatctcgttgatatcct

SEQ ID NO: 423 - DNA, 25 bp, synthetic construct
  misc_feature 1..25: note = morpholino antisense oligonucleotide
  source 1..25: mol_type = other DNA
atcatctcgttgatatcctcaaggt

SEQ ID NO: 424 - DNA, 25 bp, synthetic construct
  misc_feature 1..25: note = morpholino antisense oligonucleotide
  source 1..25: mol_type = other DNA
cttgatgatcatctcgttgatatcc

SEQ ID NO: 425 - DNA, 25 bp, synthetic construct
  misc_feature 1..25: note = morpholino antisense oligonucleotide
  source 1..25: mol_type = other DNA
accctctgtgattttataacttgat

SEQ ID NO: 426 - DNA, 25 bp, synthetic construct
  misc_feature 1..25: note = morpholino antisense oligonucleotide
  source 1..25: mol_type = other DNA
tgcttgatgatcatctcgttgatat

SEQ ID NO: 427 - DNA, 25 bp, synthetic construct
  misc_feature 1..25: note = morpholino antisense oligonucleotide
  source 1..25: mol_type = other DNA
gcttgatgatcatctcgttgatatc

SEQ ID NO: 428 - DNA, 25 bp, synthetic construct
  misc_feature 1..25: note = morpholino antisense oligonucleotide
  source 1..25: mol_type = other DNA
ccatcaccctctgtgattttataac

SEQ ID NO: 429 - DNA, 25 bp, synthetic construct
  misc_feature 1..25: note = morpholino antisense oligonucleotide
  source 1..25: mol_type = other DNA
ctgcttgatgatcatctcgttgata

SEQ ID NO: 430 - DNA, 25 bp, synthetic construct
  misc_feature 1..25: note = morpholino antisense oligonucleotide
  source 1..25: mol_type = other DNA
accatcaccctctgtgattttataa

SEQ ID NO: 431 - DNA, 25 bp, synthetic construct
  misc_feature 1..25: note = morpholino antisense oligonucleotide
  source 1..25: mol_type = other DNA
catcaccctctgtgattttataact

SEQ ID NO: 432 - DNA, 25 bp, synthetic construct
  misc_feature 1..25: note = morpholino antisense oligonucleotide
  source 1..25: mol_type = other DNA
tctgcttgatgatcatctcgttgat

SEQ ID NO: 433 - DNA, 25 bp, synthetic construct
  misc_feature 1..25: note = morpholino antisense oligonucleotide
  source 1..25: mol_type = other DNA
ttctgcttgatgatcatctcgttga

SEQ ID NO: 434 - DNA, 25 bp, synthetic construct
  misc_feature 1..25: note = morpholino antisense oligonucleotide
  source 1..25: mol_type = other DNA
cttctgcttgatgatcatctcgttg

SEQ ID NO: 435 - DNA, 25 bp, synthetic construct
  misc_feature 1..25: note = morpholino antisense oligonucleotide
  source 1..25: mol_type = other DNA
ctgtgattttataacttgatcaagc

SEQ ID NO: 436 - DNA, 25 bp, synthetic construct
  misc_feature 1..25: note = morpholino antisense oligonucleotide
  source 1..25: mol_type = other DNA
atcaccctctgtgattttataactt

SEQ ID NO: 437 - DNA, 25 bp, synthetic construct
  misc_feature 1..25: note = morpholino antisense oligonucleotide
  source 1..25: mol_type = other DNA
gtgattttataacttgatcaagcag

SEQ ID NO: 438 - DNA, 25 bp, synthetic construct
  misc_feature 1..25: note = morpholino antisense oligonucleotide
  source 1..25: mol_type = other DNA
tgattttataacttgatcaagcaga

SEQ ID NO: 439 - DNA, 25 bp, synthetic construct
  misc_feature 1..25: note = morpholino antisense oligonucleotide
  source 1..25: mol_type = other DNA
tgtgattttataacttgatcaagca

SEQ ID NO: 440 - DNA, 20 bp, synthetic construct
  misc_feature 1..20: note = Table 1. control hGAPDH_860 79_Rv
  source 1..20: mol_type = other DNA
ggaggagtgggtgtcgctgt